(19) United States
(12) Patent Application Publication
(10) Pub. No.: US 2024/0217950 A1
(43) Pub. Date: Jul. 4, 2024

(54) PESTICIDALLY ACTIVE CYCLIC AMINE COMPOUNDS

(71) Applicant: SYNGENTA CROP PROTECTION AG, Basel (CH)

(72) Inventors: Vikas SIKERVAR, Bracknell, Berkshire (GB); Ottmar Franz HUETER, Stein (CH); Myriem EL QACEMI, Stein (CH); Elke Maria HILLESHEIM, Stein (CH)

(73) Assignee: SYNGENTA CROP PROTECTION AG, Basel (CH)

(21) Appl. No.: 18/553,466

(22) PCT Filed: Mar. 24, 2022
(86) PCT No.: PCT/EP2022/057816

§ 371 Date: Sep. 29, 2023

(30) Foreign Application Priority Data
Mar. 30, 2021 (IN) 202111014417

(51) Int. Cl. C07D 401/12; A01N 43/88; A01P 7/04; C07D 413/14

(52) U.S. Cl. CPC C07D 401/12; A01N 43/88; A01P 7/04; C07D 413/14

(57) ABSTRACT

Compounds of formula (I) wherein the substituents are as defined in claim 1, and the agrochemically acceptable salts, stereoisomers, enantiomers, tautomers and N-oxides of those compounds, can be used as insecticides.

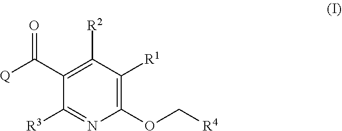

The present invention relates to pesticidally active, in particular insecticidally active cyclic amine, preferably azetidinyl-, pyrrolidinyl-, piperidinyl- and piperazinyl-pyridinyl carbonyl compounds, to processes for their preparation, to compositions comprising those compounds, and to their use for controlling animal pests, including arthropods and in particular insects or representatives of the order Acarina.

WO2015032280, CN106316931, WO2017195703, WO2019039429, WO 2019082808, JP 2019077618, JP 2019085371 and WO2021053161 describe certain azetidinyl-, pyrrolidinyl-, piperidinyl- or piperazinyl-pyridinyl carbonyl compounds for use for controlling pests that damage plants.

There have now been found novel pesticidally azetidinyl-, pyrrolidinyl-, piperdinyl- and piperazinyl-pyridinyl carbonyl compounds.

The present invention accordingly relates in a first aspect to a compound of the formula (I)

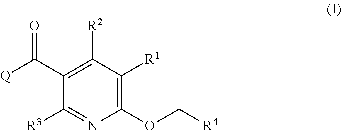

wherein
R¹ is CN or C(═S)NH₂;
R² is H, OH, halogen, C₁-C₆-alkoxy or C₁-C₆-haloalkoxy;
R³ is C₁-C₆-alkoxy-C₁-C₆-alkyl, C₁-C₆-haloalkoxy-C₁-C₆-alkyl, C₂-C₆-alkenyloxy-C₁-C₆-alkyl, C₂-C₆-haloalkenyloxy-C₁-C₆-alkyl, C₂-C₆-alkynyloxy-C₁-C₆-alkyl, C₂-C₆-haloalkynyloxy-C₁-C₆-alkyl, C₃-C₆-cycloalkoxy-C₁-C₆-alkyl, C₃-C₆-halocycloalkoxy-C₁-C₆-alkyl, C₁-C₆-alkylsulfonyl-C₁-C₆-alkyl; C₁-C₆-alkylsulfonyl-C₃-C₆-cycloalkyl, C₁-C₆-cyanoalkyl, or C₃-C₆-cyanocycloalkyl;
R⁴ is C₁-C₆-haloalkyl, C₂-C₆-alkenyl, C₂-C₆-haloalkenyl, C₃-C₆-cycloalkyl, C₃-C₆-halocycloalkyl, phenyl, phenyl substituted with 1 to 3 independently selected substituents R⁵, heteroaryl (which is either a 5 or 6 membered monocyclic or a 8, 9 or 10 membered bicyclic), heteroaryl (which is either a 5 or 6 membered monocyclic or a 8, 9 or 10 membered bicyclic) substituted with 1 to 3 independently selected substituents R⁶, phenyl-C₁-C₃-alkyl, or phenyl-C₁-C₃-alkyl substituted with 1 to 3 substituents independently selected R⁷;
Q is a cyclic amine represented by the formula IIa or a cyclic amine represented by the formula IIb,

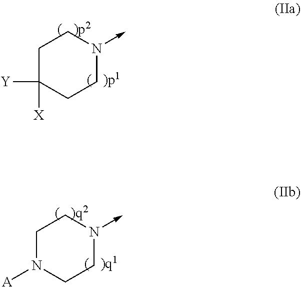

wherein the arrow indicates the connection to the carbonyl group;
p¹ is 0, 1 or 2 and indicates the number of methylene groups;
p² is 0, 1 or 2 and indicates the number of methylene groups;
q¹ is 1 or 2 and indicates the number of methylene groups;
q² is 1 or 2 and indicates the number of methylene groups;
X is hydrogen, hydroxyl, alkoxy, or halogen;
Y is cyano, C₁-C₆-alkyl, C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, C₃-C₆-alkenylsulfanyl-C₁-C₆-alkyl, C₃-C₆-alkenylsulfinyl-C₃-C₆-alkyl, C₃-C₆-alkenylsulfonyl-C₁-C₆-alkyl, C₃-C₆-alkynylsulfanyl-C₁-C₆-alkyl, C₃-C₆-alkynylsulfinyl-C₁-C₆-alkyl, C₁-C₆-alkynylsulfonyl-C₁-C₆-alkyl, R^{a}R^{b}NC(O), R^{c}C(O)NR^{d}, R^{e}SO₂NR^{f}, R^{g}O—N═CR^{h}, 4 to 6 membered non-aromatic heterocyclic ring system in which one or two carbons are replaced independently by nitrogen, oxygen, sulfur, or sulfonyl, phenyl, phenyl substituted with 1 to 3 independently selected substituents R⁸, 5 or 6 membered monocyclic heteroaryl, or 5 or 6 membered monocyclic heteroaryl substituted with 1 to 3 independently selected substituents R⁹;
A is cyano, C₁-C₆-cyanoalkyl, C₂-C₆-cyanoalkenyl, C₃-C₆-cyanocycloalkyl, C₁-C₆-haloalkyl, C₁-C₆-haloalkenyl, C₃-C₆-halocycloalkyl, C₁-C₆-alkoxycarbonyl, C₂-C₆-alkenyloxycarbonyl, C₂-C₆-alkynyloxycarbonyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, RⁱSO₂, R^{j}R^{k}NSO₂, phenyl, phenyl substituted with 1 to 3 independently selected substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 independently selected substituents R¹¹;
R^{a}, R^{b}, R^{c}, R^{d}, R^{f}, R^{g}, R^{h}, R^{j} and R^{k} are independently selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl and C₃-C₆-halocycloalkyl;
R^{e} and Rⁱ are independently selected from C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl and C₃-C₆-halocycloalkyl;
R⁵ is independently selected from halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₁-C₆-alkylsulfonyl, C₁-C₆-haloalkylsulfanyl and —O—C₁₋₂haloalkanediyl-O—; and
R⁶, R⁷, R⁸, R⁹, R¹⁰, R¹¹ are independently selected from halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₁-C₆-alkylsulfonyl, and C₁-C₆-haloalkylsulfanyl;
or an agrochemically acceptable salt, stereoisomer, enantiomer, tautomer and N-oxide of the compound of formula (I).

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

The term “C₁-Cₙ-alkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₂-Cₙ-alkenyl” as used herein refers to a straight or branched alkenyl chain having form two to n carbon atoms and one or two double bonds, for example, ethenyl, prop-I-enyl, but-2-enyl.

The term “C₂-Cₙ-alkynyl” as used herein refers to a straight or branched alkynyl chain having from two to n carbon atoms and one triple bond, for example, ethynyl, prop-2-ynyl, but-3-ynyl,

The term “C₃-Cₙ-cycloalkyl” as used herein refers to 3-n membered cycloalkyl groups such as cyclopropane, cyclobutane, cyclopropane, cyclopentane and cyclohexane.

The term “C₁-Cₙ-alkoxy” as used herein refers to a straight-chain or branched saturated alkyl radical having 1 to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy or 1,1-dimethylethoxy. The term “haloC₁-Cₙ-alkoxy” as used herein refers to a C₁-Cₙ-alkoxy radical where one or more hydrogen atoms on the alkyl radical is replaced by the same or different halo atom(s)—examples include trifluoromethoxy, 2-fluoroethoxy, 3-fluoropropoxy, 3,3,3-trifluoropropoxy, 4-chlorobutoxy. Two neighboring substituents of a phenyl ring may form together with the carbons of the phenyl ring a 5- or 6-membered ring. Examples are —OCF₂O—, —OCF₂CF₂O—.

Halogen is generally fluorine, chlorine, bromine or iodine. This also applies, correspondingly, to halogen in combination with other meanings, such as haloalkyl.

The term “C₁-Cₙ-haloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl. Similarly, the term “C₂-Cₙ-haloalkenyl” or “C₂-Cₙ-haloalkynyl” as used herein refers to a C₂-Cₙ-alkenyl or C₂-Cₙ-alkynyl moiety respectively substituted with one or more halo atoms which may be the same or different. Similarly, the term “C₃-Cₙ-halocycloalkyl” as used herein refers to a C₃-Cₙ-cycloakyl group substituted with one or more halo atoms which may be the same or different.

The term “C₁-Cₙ-cyanoalkyl” as used herein refers to C₁-Cₙ-alkyl radical having 1 to n carbon atoms (as mentioned above), where one of the hydrogen atoms in the radical is be replaced by a cyano group: for example, cyanomethyl, 2-cyanoethyl, 2-cyanopropyl, 3-cyanopropyl, 1-(cyanomethyl)-2-ethyl, 1-(methyl)-2-cyanoethyl, 4-cyanobutyl, and the like. Similarly, the term “C₁-Cₙ-cyanoalkenyl” or “C₁-Cₙ-cyanoalkynyl” refers to a C₂-Cₙ-alkenyl or C₁-Cₙ-alkynyl moiety respectively substituted with one of the hydrogen atoms in the corresponding moiety being replaced by a cyano group.

The term “C₁-Cₙ-alkylsulfanyl-C₁-Cₙ-alkyl” as used herein refers to an alkyl radical wherein one of the non-terminal carbon atoms is replaced by a sulfur atom.

The term “C₁-Cₙ-alkylsulfinyl-C₁-Cₙ-alkyl” as used herein refers to an alkyl radical wherein one of the non-terminal carbon atoms is replaced by a S(═O) group.

The term “C₁-Cₙ-alkylsulfonyl-C₁-Cₙ-alkyl” as used herein refers to an alkyl radical wherein one of the non-terminal carbon atoms is replaced by a S(═O)₂ group.

The term “C₁-Cₙ-alkylsulfonyl-C₃-Cₙ-cycloalkyl” as used herein refers to a C₃-Cₙ-cycloalkyl radical substituted with a C₁-Cₙalkylsulfonyl group.

The term “4 to 6 membered non-aromatic heterocyclic ring system in which one or two carbons is replaced by nitrogen, oxygen, sulfur, or sulfonyl,” as used herein refers to a cyclic group where one or two carbon atoms in the ring are replaced independently by nitrogen, oxygen, sulfur, or sulfonyl, and the ring is attached via a carbon, or a nitrogen atom to remainder of the compound. Examples are azetidinyl, oxetanyl, thietanyl, pyrrolidinyl, tetrahydrofuranyl, 2-oxopyrrolidinyl, 2-oxotetrahydrofuranyl, 1,1-dioxo-1,2-thiazolidinyl, 1,3-dioxolanyl, 1,3-dithiolanyl, 2-oxooxazolidinyl, piperidinyl, tetrahydropyranyl, 2-oxopiperidinyl, 1,1-dioxothiazinanyl, 2-oxotetrahydropyranyl, 1,3-dioxolanyl, 1,3-dithianyl, 2-oxo-1,3-oxazinanyl.

The term “phenyl-C₁-Cₙ-alkyl” as used herein refers to an alkyl radical substituted with a phenyl ring. If there is substitution on the phenyl-C₁-Cₙ-alkyl group, the substitution is on the phenyl ring.

The term “5 or 6 membered monocyclic heteroaryl” as used herein refers to a 5 or 6 membered aromatic ring having 1 to 3 carbon atoms replaced independently by nitrogen, sulfur, or oxygen.

Examples are pyridyl (or pyridinyl), pyridazinyl, pyrimidinyl, pyrazinyl, pyrrolyl, pyrazolyl, imidazolyl, triazolyl (e.g. 1.2.4 triazoyl), furanyl, thiophenyl, oxazolyl, isoxazolyl, oxadiazolyl, thiazolyl, isothiazolyl and thiadiazolyl.

The term “8, 9 or 10 membered bicyclic heteroaryl” as used herein refers to a 8, 9 or 10 membered aromatic ring made up of two rings, having 1 to 4 carbon atoms replaced independently by nitrogen, sulfur, or oxygen (the heteroatoms can be in one ring or distributed amongst the two). Examples are purinyl, quinolinyl, isoquinolinyl, cinnolinyl, quinoxalinyl, indolyl, indazolyl, 2,1,3-benzoxadiazolyl, 2,1,3-benzothiadiazolyl, benzimidazolyl, benzothiophenyl, benzoxazolyl, benzothiazolyl, imidazo[1,2-a]pyridinyl, imidazo[4,5-b]pyridinyl, imidazo[2,1-b]thiazole and imidazo[2,1-b][1,3,4]thiadiazolyl.

The term “C₁-Cₙ-alkoxy-C₁-Cₙ-alkyl” as used herein refers to an alkyl radical substituted with C₁-Cₙ-alkoxy group. Examples are methoxymethyl, methoxyethyl, ethoxymethyl and propoxymethyl.

The term “C₁-Cₙ-haloalkoxy-C₁-Cₙ-alkyl” as used herein refers to an alkyl radical substituted with C₁-Cₙ-haloalkoxy group. Examples are trifluoromethoxymethyl, and chloromethoxymethyl.

The terms “C₂-Cₙ-alkenyloxy-C₁-Cₙ-alkyl” and “C₂-Cₙ-haloalkenyloxy-C₁-Cₙ-alkyl” as used herein refers to a C₂-Cₙ-alkenyl group and C₂-Cₙ-haloalkenyl group respectively connected via an oxygen atom to a C₁-Cₙ-alkyl radical, and where the attachment to the remainder of the compound is through the carbon atom of the alkyl radical.

The terms “C₂-Cₙ-alkynyloxy-C₁-Cₙ-alkyl” and “C₂-Cₙ-haloalkynyloxy-C₁-Cₙ-alkyl” as used herein refers to a C₂-Cₙ-alkynyl group and C₂-Cₙ-haloalkynyl group respectively connected via an oxygen atom to a C₁-Cₙ-alkyl radical, and where the attachment to the remainder of the compound is through the carbon atom of the alkyl radical.

The terms “C₃-Cₙ-cycloalkoxy-C₁-Cₙ-alkyl” and “C₃-Cₙ-halocycloalkoxy-C₁-Cₙ-alkyl” as used herein refers to an alkyl radical substituted with a “C₃-Cₙ-cycloalkoxy” and “C₃-Cₙ-halocycloalkoxy” group respectively.

The term “C₁-Cₙ-alkoxycarbonyl” as used herein refers to a C₁-Cₙ-alkoxy group connected to a carbonyl (C═O) radical, through which the C₁-Cₙ-alkoxycarbonyl is attached to the remainder of the compound. Examples are methoxycarbonyl, ethoxycarbonyl, isopropoxycarbonyl and tertiary butyloxycarbonyl.

The term “C₂-Cₙ-alkenyloxycarbonyl” as used herein refers to a C₂-Cₙ-alkenyl group connected with an oxygen atom to a carbonyl (C═O) radical, and where the attachment to the remainder of the compound is through the carbon atom of the carbonyl. Examples are vinyloxycarbonyl and allyloxycarbonyl.

The term “C₂-Cₙ-alkynyloxycarbonyl” as used herein refers to a C₂-Cₙ-alkynyl group connected with an oxygen atom to a carbonyl (C═O) radical, and where the attachment to the remainder of the compound is through the carbon atom of the carbonyl. Examples are propargyloxycarbonyl (2-prop-2-ynoxycarbonyl) and 2-pent-3-ynoxycarbonyl,

The term “—O—C₁₋₂haloalkanediyl-O—” as used herein refers to adjacent positions on the phenyl ring being connected to the oxygen atoms of the —O—C₁₋₂haloalkanediyl-O— group and the oxygen atoms being linked by 1 to 2 carbon atoms substituted with one or more halogen atoms. Examples are OCF₂O— and —OCF₂CF₂O—.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, by the use of known techniques and by observing results obtained under analogous circumstances. In determining the effective amount a number of factors are considered including, but not limited to: the type of plant or derived product to be applied; the pest to be controlled & its lifecycle; the particular compound applied; the type of application; and other relevant circumstances.

Embodiments according to the invention are provided as set out below.

In an embodiment of each aspect of the invention, R¹ is
A. CN or C(═S)NH₂; or
B. CN.

In an embodiment of each aspect of the invention, R² is
A. hydrogen, halogen, C₁-C₆-alkoxy or C₁-C₆-haloalkoxy; or
B. hydrogen

In an embodiment of each aspect of the invention, R³ is
A. C₁-C₄-alkoxy-C₁-C₃-alkyl, C₁-C₄-haloalkoxy-C₁-C₃-alkyl, C₂-C₄-alkenyloxy-C₁-C₃-alkyl, C₂-C₄-haloalkenyloxy-C₁-C₃-alkyl, C₂-C₄-alkynyloxy-C₁-C₃-alkyl, C₂-C₄-haloalkynyloxy-C₁-C₃-alkyl, C₃-C₆-cycloalkoxy-C₁-C₃-alkyl, C₃-C₆-halocycloalkoxy-C₁-C₃-alkyl, C₁-C₄-alkylsulfonyl-C₁-C₃-alkyl; C₁-C₄-alkylsulfonyl-C₃-C₆-cycloalkyl, C₁-C₄-cyanoalkyl, or C₃-C₆-cyanocycloalkyl; or
B. C₁-C₃-alkoxy-C₁-C₃-alkyl, C₁-C₃-alkylsulfonyl-C₁-C₃-alkyl; C₁-C₃-alkylsulfonyl-C₃-C₃-cycloalkyl, C₁-C₃-cyanoalkyl, or C₃-C₆-cyanocycloalkyl; or
C. C₁-C₃-alkoxy-C₁-C₃-alkyl, C₁-C₃-alkylsulfonyl-C₁-C₃-alkyl; C₁-C₃-alkylsulfonyl-cyclopropyl, C₁-C₃-cyanoalkyl, or cyano-cyclopropyl; or
D. C₁-C₂-alkoxy-C₁-C₂-alkyl, C₁-C₂-alkylsulfonyl-C₁-C₂-alkyl; C₁-C₂-alkylsulfonyl-cyclopropyl, C₁-C₂-cyanoalkyl, or cyano-cyclopropyl; or
E. methoxymethyl, ethoxymethyl, methysulfonylmethyl, methysulfonylcyclopropyl, cyano-methyl or cyano-cyclopropyl; or
F. methoxymethyl, methysulfonylmethyl, or cyano-methyl; or
G. methoxymethyl.

In an embodiment of each aspect of the invention, R⁴ is
A. C₁-C₆-haloalkyl, C₂-C₆-alkenyl, C₂-C₆-haloalkenyl, C₃-C₆-cycloalkyl, C₃-C₆-halocycloalkyl, phenyl, phenyl substituted with 1 to 3 independently selected substituents R⁵, 5 or 6 membered monocyclic heteroaryl, 5 or 6 membered monocyclic heteroaryl optionally substituted with 1 to 3 independently selected substituents R⁶, 8, 9 or 10 membered bicyclic heteroaryl optionally substituted with 1 to 3 independently selected substituents R⁶, phenyl-C₁-C₃-alkyl, or phenyl-C₁-C₃-alkyl substituted with 1 to 3 independently selected substituents R⁷; or
B. C₁-C₆-haloalkyl, C₂-C₆-haloalkenyl, C₃-C₆-cycloalkyl or C₃-C₆-halocycloalkyl, phenyl substituted with 1 to 3 independently selected substituents R⁵, 5 or 6 membered monocyclic heteroaryl, 5 or 6 membered monocyclic heteroaryl substituted with 1 to 3 independently selected substituents R⁶, 8, 9 or 10 membered bicyclic heteroaryl optionally substituted with 1 to 3 independently selected substituents R⁶, phenyl-C₁-C₃-alkyl, or phenyl-C₁-C₃-alkyl substituted with 1 to 3 independently selected substituents R⁷; or
C. phenyl substituted with 1 to 3 independently selected substituents R⁵, 5 or 6 membered monocyclic heteroaryl substituted with 1 to 3 independently selected substituents R⁶, or 8, 9 or 10 membered bicyclic heteroaryl optionally substituted with 1 to 3 independently selected substituents R⁶; or
D. phenyl substituted with 1 to 3 substituents independently selected from halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₁-C₆-alkylsulfonyl, and C₁-C₆-haloalkylsulfanyl, or one of thiophenyl, thiazolyl, thiadiazolyl, pyridyl (or pyridinyl), pyrazolyl, 2,1,3-benzoxadiazolyl, and 1,3-benzodioxolyl—independent of each other, is unsubstituted or substituted with 1 to 3 substituents independently selected from halogen, C₁-C₃-alkyl, C₁-C₃-haloalkyl, C₁-C₃-alkoxy; and C₁-C₃-haloalkoxy; or
E. phenyl substituted with 1 to 3 substituents independently selected from halogen, cyano, C₁-C₆-haloalkyl, C₁-C₆-haloalkoxy, C₁-C₆-alkylsulfonyl, C₁-C₆-haloalkylsulfanyl and —O—C₁₋₂haloalkanediyl-O—, or one of thiophenyl, thiazolyl, thiadiazolyl, pyridyl (or pyridinyl), pyrazolyl, 2,1,3-benzoxadiazolyl, and 1,3-benzodioxolyl—independent of each other, is unsubstituted or substituted with 1 to 3 substituents independently selected from fluorine, chlorine, methyl, ethyl, isopropyl, trifluoromethyl, difluoromethyl, trifluoromethoxy, and difluoromethoxy; or
F. phenyl substituted with 1 or 2 substituents independently selected from halogen, cyano, C₁-C₆-alkyl, C₁-C₆-haloalkyl, C₁-C₆-haloalkoxy, C₁-C₆-alkylsulfonyl, C₁-C₆-haloalkylsulfanyl and —O—C₁₋₂haloalkanediyl-O—, or one of thiophenyl, pyridyl (or pyridinyl), and pyrazolyl—each substituted with 1 to 3 substituents independently selected from fluorine, chlorine, methyl, ethyl, isopropyl, trifluoromethyl, difluoromethyl, trifluoromethoxy, and difluoromethoxy; or
G. phenyl substituted with 1 or 2 substituents independently selected from halogen, cyano, C₁-C₆-haloalkoxy, C₁-C₆-haloalkyl and —O—C₁₋₂haloalkanediyl-O—, or one of thiophenyl, pyridyl (or pyridinyl), and pyrazolyl—each substituted with 1 to 2 substituents independently selected from halogen, C₁-C₆-haloalkoxy, C₁-C₆-alkyl and C₁-C₆-haloalkyl; or
H. phenyl substituted with a halogen, cyano, C₁-C₃-alkylsulfonyl, C₁-C₃-haloalkylsulfanyl, C₁-C₃-haloalkoxy, C₁-C₃-haloalkyl and —O—C₁₋₂haloalkanediyl-O—, or one of thiophenyl, pyridyl (or pyridinyl), and pyrazolyl—each substituted with 1 to 2 substituents independently selected from halogen, C₁-C₃-haloalkoxy, C₁-C₃-alkyl and C₁-C₃-haloalkyl; or
I. phenyl substituted with one to three substituents independently selected from halogen, cyano, C₁-C₃-alkylsulfonyl, C₁-C₃-haloalkylsulfanyl, C₁-C₃-haloalkoxy, C₁-C₃-haloalkyl and —O—C₁₋₂haloalkanediyl-O—; or
J. phenyl substituted with one to three substituents independently selected from halogen, cyano, C₁-C₃-haloalkoxy, C₁-C₃-haloalkyl and —O—C₁₋₂haloalkanediyl-O—; or
K. one of thiophenyl, pyridyl (or pyridinyl), and pyrazolyl—each substituted with 1 to 2 substituents independently selected from halogen, C₁-C₃-haloalkoxy, C₁-C₃-alkyl and C₁-C₃-haloalkyl; or 2,1,3-benzoxadiazolyl.

In an embodiment of each aspect of the invention, Q is
A. a cyclic amine represented by the formula IIa, where both p¹ and p² are 1; X is hydrogen, hydroxyl, alkoxy, or halogen; and Y is cyano, C₁-C₆-alkyl, C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, R^{a}R^{b}NC(O), R^{c}C(O)NR^{d}, R^{e}SO₂NR^{f}, R^{g}O—N═CR^{h}, 4 to 6 membered non-aromatic heterocyclic ring system in which one or two carbons is replaced independently by nitrogen, oxygen, sulfur, or sulfonyl, phenyl, phenyl substituted with 1 to 3 substituents R⁸, a 5 or 6 membered monocyclic heteroaryl, or a 5 or 6 membered monocyclic heteroaryl substituted with 1 to 3 substituents R⁹;

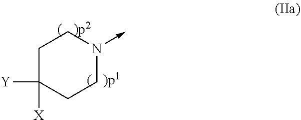

B. a cyclic amine represented by the formula IIa, where both p¹ and p² are 1; X is hydrogen or hydroxyl; and Y is cyano, C₁-C₆-alkyl, C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, R^{a}R^{b}NC(O), R^{c}C(O)NR^{d},
R^{e}SO₂NR^{f}, R^{g}O—N═CR^{h}, 4 to 6 membered non-aromatic heterocyclic ring system in which one or two carbons is replaced independently by nitrogen, oxygen, sulfur or sulfonyl, phenyl, phenyl substituted with 1 to 3 substituents R⁸, 5 or 6 membered monocyclic heteroaryl, or a 5 or 6 membered monocyclic heteroaryl substituted with 1 to 3 substituents R⁹; or
C. a cyclic amine represented by the formula IIa, where both p¹ and p² are 1, X is hydrogen or hydroxyl; and Y is cyano, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, R^{a}R^{b}NC(O), R^{e}SO₂NR^{f}, R^{g}O—N═CR^{h}, 4 to 6 membered non-aromatic heterocyclic ring system in which one or two carbons is replaced independently by nitrogen, oxygen, sulfur or sulfonyl, phenyl, phenyl substituted with 1 to 3 substituents R⁸, 5 or 6 membered monocyclic heteroaryl, or a 5 or 6 membered monocyclic heteroaryl substituted with 1 to 3 substituents R⁹; or
D. a cyclic amine represented by the formula IIa, where both p¹ and p² are 1, X is hydrogen or hydroxyl; and Y is cyano, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, 4 to 6 membered non-aromatic heterocyclic ring system in which one or two carbons is replaced independently by nitrogen, oxygen, sulfur or sulfonyl, 5 or 6 membered monocyclic heteroaryl, or 5 or 6 membered monocyclic heteroaryl substituted with 1 to 3 substituents R⁹; or
E. a cyclic amine represented by the formula IIa, where both p¹ and p² are 1, X is hydrogen or hydroxyl; and Y is 5 or 6 membered monocyclic heteroaryl, or 5 or 6 membered monocyclic heteroaryl substituted with 1 to 3 substituents R⁹; or
F. a cyclic amine represented by the formula IIa, where both p¹ and p² are 1, X is hydrogen or hydroxyl; and Y is 5 or 6 membered monocyclic heteroaryl substituted with 1 to 3 substituents independently selected from halogen, C₁-C₆-alkyl, and C₁-C₆-haloalkyl; or
G. a cyclic amine represented by the formula IIa, where both p¹ and p² are 1, X is hydrogen or hydroxyl; and Y is C₁-C₃-alkoxy-C₁-C₃-alkyl, C₁-C₃-alkylsulfanyl-C₁-C₃-alkyl, C₁-C₃-alkylsulfinyl-C₁-C₃-alkyl, or C₁-C₃-alkylsulfonyl-C₁-C₃-alkyl; or
H. a cyclic amine represented by the formula IIa, where both p¹ and p² are 1, X is hydrogen; and Y is methoxymethyl, ethylsulfanylmethyl, ethylsulfinylmethyl, ethylsulfonylmethyl, 1-methyl-1,2,4-triazole-3-yl, 1,1-dioxo-1,2-thiazolidin-2-yl, 1-methylpyrazol-3-yl, 5-methyl-1,1-dioxo-1,2,5-thiadiazolidine-2-yl, or 5-ethyl-1,1-dioxo-1,2,5-thiadiazolidine-2-yl; or
I. cyclic amine represented by the formula IIb; where both q¹ and q² are 1; and A is cyano, C₁-C₆-cyanoalkyl, C₂-C₆-cyanoalkenyl, C₃-C₆-cyanocycloalkyl, C₁-C₆-haloalkyl, C₁-C₆-haloalkenyl, C₃-C₆-halocycloalkyl, C₁-C₆-alkoxycarbonyl, C₂-C₆-alkenyloxycarbonyl, C₂-C₆-alkynyloxycarbonyl, RⁱSO₂, R^{j}R^{k}NSO₂, phenyl, phenyl substituted with 1 to 3 substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 substituents R¹¹; or

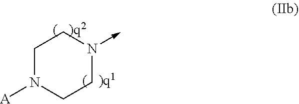

J. cyclic amine represented by the formula IIb; where both q¹ and q² are 1; and A is cyano, C₁-C₆-cyanoalkyl, C₁-C₆-haloalkyl, C₁-C₆-alkoxycarbonyl, C₂-C₆-alkenyloxycarbonyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, phenyl, phenyl substituted with 1 to 3 substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 substituents R¹¹; or
K. cyclic amine represented by the formula IIb; where both q¹ and q² are 1; and A is RⁱSO₂, R^{j}R^{k}NSO₂, phenyl, phenyl substituted with 1 to 3 substituents R¹⁰, 5 or 6 membered monocyclic heteroaryl, or 5 or 6 membered monocyclic heteroaryl substituted with 1 to 3 substituents R¹¹; or
L. cyclic amine represented by the formula IIb; where both q¹ and q² are 1; and A is C₁-C₃-alkylsulfonyl, (C₁-C₃-alky)HNSO₂, (C₁-C₃-alky)₂NSO₂, phenyl substituted with 1 to 3 substituents independently selected from halogen, C₁-C₃-alkyl and C₁-C₃-haloalkyl, or 5 or 6 membered monocyclic heteroaryl substituted with 1 to 3 substituents independently selected from halogen, C₁-C₃-alkyl and C₁-C₃-haloalkyl; or
M. cyclic amine represented by the formula IIb; where both q¹ and q² are 1; and A is ethylsulfonyl, propylsulfonyl, iso-propylsulfonyl, cyclopropylsulfonyl, methylsulfamoyl, ethylsulfamoyl, dimethylsulfamoyl, ethyl(methyl)sulfamoyl, 4-fluoro-phenyl, 3,5-dichloropyridine-2-yl, 4-fluoro-pyridine-2-yl, 5-methyl-1,2,4-oxadiazol-3-yl, 3-methyl-1,2,4-oxadiazol-5-yl, or 1-methylpyrazol-3-yl.

In an embodiment of each aspect of the invention, R^{a}, R^{b}, R^{c}, R^{d}, R^{f}, R^{g}, R^{h}, R^{j} and R^{k} are independently selected from
A. hydrogen, C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl and C₃-C₄-halocycloalkyl; or
B. hydrogen, C₁-C₃-alkyl, C₁-C₃-haloalkyl, C₃-C₄-cycloalkyl and C₃-C₄-halocycloalkyl; or
C. hydrogen, C₁-C₃-alkyl, C₁-C₃-haloalkyl, and C₃-C₄-halocycloalkyl; or
D. hydrogen, C₁-C₃-alkyl and C₁-C₃-haloalkyl; or
E. hydrogen and C₁-C₃-haloalkyl.

In an embodiment of each aspect of the invention, R^{e} and Rⁱ are independently selected from
A. C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl and C₃-C₄-halocycloalkyl; or
B. C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl and C₃-C₄-halocycloalkyl; or
C. C₁-C₃-alkyl and C₁-C₃-haloalkyl.

In an embodiment of each aspect of the invention, R⁶, R⁷, R⁸, R⁹, R¹⁰, R¹¹ are independently selected from
A. halogen, cyano, C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl, C₃-C₄-halocycloalkyl, C₁-C₃-alkoxy; C₁-C₃-haloalkoxy; C₁-C₃-alkylsulfonyl, and C₁-C₃-haloalkylsulfanyl; or
B. halogen, cyano, C₁-C₃-alkyl, C₁-C₃-haloalkyl, C₃-C₄-cycloalkyl, C₃-C₄-halocycloalkyl, C₁-C₃-alkoxy; C₁-C₃-haloalkoxy; C₁-C₃-alkylsulfonyl, and C₁-C₃-haloalkylsulfanyl; or
C. halogen, cyano, C₁-C₃-alkyl, C₁-C₃-haloalkyl, C₃-C₄-cycloalkyl, C₃-C₄-halocycloalkyl, C₁-C₃-alkoxy; and C₁-C₃-haloalkoxy; or
D. halogen, C₁-C₃-alkyl, C₁-C₃-haloalkyl, C₁-C₃-alkoxy; and C₁-C₃-haloalkoxy; or
E. halogen, C₁-C₃-alkyl, C₁-C₃-haloalkyl, C₁-C₃-alkoxy; and C₁-C₃-haloalkoxy; or
F. halogen, C₁-C₃-alkyl, and C₁-C₃-haloalkyl and C₁-C₃-haloalkoxy; or
G. fluorine, bromine, chlorine, methyl, ethyl, isopropyl, trifluoroethyl, trifluoromethyl, difluoromethyl, trifluoromethoxy, and difluoromethoxy.

In an embodiment of each aspect of the invention, R⁵ is independently selected from
A. halogen, cyano, C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl, C₃-C₄-halocycloalkyl, C₁-C₃-alkoxy; C₁-C₃-haloalkoxy; C₁-C₃-alkylsulfonyl, C₁-C₃-haloalkylsulfanyl and —O—C₁₋₂haloalkanediyl-O—; or
B. halogen, cyano, C₁-C₃-alkyl, C₁-C₃-haloalkyl, C₃-C₄-cycloalkyl, C₃-C₄-halocycloalkyl, C₁-C₃-alkoxy; C₁-C₃-haloalkoxy; C₁-C₃-alkylsulfonyl, C₁-C₃-haloalkylsulfanyl and —OC₁-C₂-haloalkylO—; or
C. halogen, cyano, C₁-C₃-alkyl, C₁-C₃-haloalkyl, C₃-C₄-cycloalkyl, C₃-C₄-halocycloalkyl, C₁-C₃-alkoxy, C₁-C₃-haloalkoxy and —OC₁-C₂-haloalkylO—; or
D. halogen, C₁-C₃-alkyl, C₁-C₃-haloalkyl, C₁-C₃-alkoxy; C₁-C₃-haloalkoxy and —OC₁-C₂-haloalkylO—; or
E. halogen, C₁-C₃-alkyl, C₁-C₃-haloalkyl, C₁-C₃-alkoxy; C₁-C₃-haloalkoxy and —OC₁-C₂-haloalkylO—; or
F. halogen, C₁-C₃-alkyl, and C₁-C₃-haloalkyl, C₁-C₃-haloalkoxy and —OC₁-C₂-haloalkylO—; or
G. fluorine, bromine, chlorine, methyl, ethyl, isopropyl, trifluoroethyl, trifluoromethyl, difluoromethyl, trifluoromethoxy, difluoromethoxy, —OCF₂O— and —OCF₂CF₂O—.

The present invention, accordingly, makes available a compound of formula (I) having the substituents
R¹, R², R³, R⁴ and Q as defined above in all combinations/each permutation. Accordingly, made available, for example, is a compound of formula (I) with R¹ being of the first aspect (i.e. R¹ is CN or C(═S)NH₂; R² being embodiment B (i.e. R² is hydrogen); R³ being embodiment A (i.e. R³ is C₁-C₄-alkoxy-C₁-C₃-alkyl, C₁-C₄-haloalkoxy-C₁-C₃-alkyl, C₂-C₄-alkenyloxy-C₁-C₃-alkyl, C₂-C₄-haloalkenyloxy-C₁-C₃-alkyl, C₂-C₄-alkynyloxy-C₁-C₃-alkyl, C₂-C₄-haloalkynyloxy-C₁-C₃-alkyl, C₃-C₆-cycloalkoxy-C₁-C₃-alkyl, C₃-C₆-halocycloalkoxy-C₁-C₃-alkyl, C₁-C₄-alkylsulfonyl-C₁-C₃-alkyl; C₁-C₄-alkylsulfonyl-C₃-C₆-cycloalkyl, C₁-C₄-cyanoalkyl, or C₃-C₆-cyanocycloalkyl); R⁴ being embodiment C (i.e. R⁴ is phenyl substituted with 1 to 3 independently selected substituents R⁵, 5 or 6 membered monocyclic heteroaryl substituted with 1 to 3 independently selected substituents R⁶, or 8, 9 or 10 membered bicyclic heteroaryl optionally substituted with 1 to 3 independently selected substituents R⁶); Q being embodiment C (i.e. Q is a cyclic amine represented by the formula IIa, where both p¹ and p² are 1, X is hydrogen or hydroxyl; and Y is cyano, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, R^{a}R^{b}NC(O), R^{e}SO₂NR^{f}, R^{g}O—N═CR^{h}, 4 to 6 membered non-aromatic heterocyclic ring system in which one or two carbons is replaced independently by nitrogen, oxygen, sulfur or sulfonyl, phenyl, phenyl substituted with 1 to 3 independently selected substituents R⁸, 5 or 6 membered monocyclic heteroaryl, or a 5 or 6 membered monocyclic heteroaryl substituted with 1 to 3 independently selected substituents R⁹); where R^{a} is embodiment C (i.e. R^{a} is hydrogen, C₁-C₃-alkyl, C₁-C₃-haloalkyl, or C₃-C₄-halocycloalkyl); R^{b} is embodiment D (i.e. R^{b} is hydrogen, C₁-C₃-alkyl or C₁-C₃-haloalkyl); R^{e} is embodiment C (i.e. R^{e} is C₁-C₃-alkyl or C₁-C₃-haloalkyl); R^{f} is embodiment A (i.e. R^{f} is hydrogen, C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl and C₃-C₄-halocycloalkyl); R⁹ is embodiment A (i.e. R⁹ is hydrogen, C₁-C₃-alkyl, C₁-C₃-haloalkyl, C₂-C₄-alkenyl, C₂-C₄-haloalkenyl, C₂-C₄-alkynyl, C₂-C₄-haloalkynyl, C₃-C₄-cycloalkyl and C₃-C₄-halocycloalkyl); R^{h} is embodiment C (i.e. R^{h} is hydrogen, C₁-C₃-alkyl, C₁-C₃-haloalkyl, or C₃-C₄-halocycloalkyl); R⁵ is embodiment C (i.e. R⁵ is independently selected from halogen, cyano, C₁-C₃-alkyl, C₁-C₃-haloalkyl, C₃-C₄-cycloalkyl, C₃-C₄-halocycloalkyl, C₁-C₃-alkoxy, C₁-C₃-haloalkoxy and —OC₁-C₂-haloalkylO—; R⁸ is embodiment E (i.e. R⁸ is independently selected from halogen, C₁-C₃-alkyl, C₁-C₃-haloalkyl, C₁-C₃-alkoxy; and C₁-C₃-haloalkoxy); and R⁹ is embodiment F (i.e. R⁹ is independently selected from fluorine, bromine, chlorine, methyl, ethyl, isopropyl, trifluoroethyl, trifluoromethyl, difluoromethyl, trifluoromethoxy, and difluoromethoxy).

In an embodiment of each aspect of the invention, the compound of the formula (I) has
R¹ is CN, or C(═S)NH₂; preferably CN;
R² is H, halogen, C₁-C₆-alkoxy or C₁-C₆-haloalkoxy; preferably hydrogen;
R³ is C₁-C₆-alkoxy-C₁-C₆-alkyl, C₁-C₆-haloalkoxy-C₁-C₆-alkyl, C₂-C₆-alkenyloxy-C₁-C₆-alkyl, C₂-C₆-haloalkenyloxy-C₁-C₆-alkyl, C₂-C₆-alkynyloxy-C₁-C₆-alkyl, C₂-C₆-haloalkynyloxy-C₁-C₆-alkyl, C₃-C₆-cycloalkoxy-C₁-C₆-alkyl, C₃-C₆-halocycloalkoxy-C₁-C₆-alkyl, C₁-C₆-alkylsulfonyl-C₁-C₆-alkyl; C₁-C₆-alkylsulfonyl-C₃-C₆-cycloalkyl, C₁-C₆-cyanoalkyl, or C₃-C₆-cyanocycloalkyl;
R⁴ is phenyl, phenyl substituted with 1 to 3 independently selected substituents R⁵, heteroaryl (which is either a 5 or 6 membered monocyclic or a 8, 9 or 10 membered bicyclic), heteroaryl (which is either a 5 or 6 membered monocyclic or a 8, 9 or 10 membered bicyclic) substituted with 1 to 3 independently selected substituents R⁶;
Q is a cyclic amine represented by the formula IIa or a cyclic amine represented by the formula IIb,

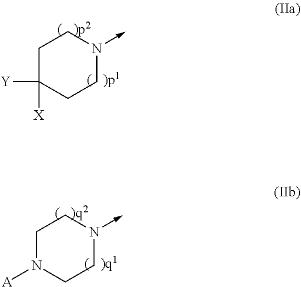

wherein the arrow indicates the connection to the carbonyl group;
p¹ is 0, or 1 and indicates the number of methylene groups;
p² is 0, or 1 and indicates the number of methylene groups;
q¹ is 1 and indicates the number of methylene groups;
q² is 1 and indicates the number of methylene groups;
X is hydrogen or hydroxyl; preferably hydrogen;
Y is C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, C₁-C₆-alkylsulfonyl(C₁-C₆-alkyl)amino, C₁-C₆-alkoxy-N═C₁-C₆-alkyl, 4 to 6 membered non-aromatic heterocyclic ring system in which one or two carbons are replaced independently by nitrogen and sulfonyl, phenyl, phenyl substituted with 1 to 3 independently selected substituents R⁸, 5 or 6 membered monocyclic heteroaryl, or 5 or 6 membered monocyclic heteroaryl substituted with 1 to 3 independently selected substituents R⁹;
A is C₁-C₃-alkylamino-sulfonyl, di(C₁-C₃-alkyl)amino-sulfonyl, phenyl, phenyl substituted with 1 to 3 independently selected substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 independently selected substituents R¹¹;
R⁵ is independently selected from halogen, cyano, C₁-C₆-haloalkyl, C₁-C₆-alkoxy, C₁-C₆-haloalkoxy, C₁-C₆-alkylsulfonyl, C₁-C₆-haloalkylsulfanyl and —O—C₁₋₂haloalkanediyl-O—; and
R⁶ is independently selected from halogen, C₁-C₆-alkyl, C₁-C₆-haloalkyl, C₁-C₆-alkoxy, C₁-C₆-haloalkoxy, C₁-C₆-alkylsulfonyl, and C₁-C₆-haloalkylsulfanyl;
R⁸ is independently selected from halogen, C₁-C₆-alkyl, C₁-C₆-haloalkyl, C₁-C₆-alkoxy, C₁-C₆- and haloalkoxy;
R⁹ is independently selected from halogen, C₁-C₆-alkyl, and C₁-C₆-haloalkyl;
R¹⁰ is independently selected from halogen, C₁-C₆-alkyl, C₁-C₆-haloalkyl, C₁-C₆-alkoxy, and C₁-C₆-haloalkoxy; and
R¹¹ is independently selected from halogen, C₁-C₆-alkyl, and C₁-C₆-haloalkyl.

In an embodiment of each aspect of the invention, the compound of formula (I) has as R¹ cyano or C(═S)NH₂, as R² hydrogen, as R³ selected from C₁-C₂-alkoxy-C₁-C₂-alkyl, C₁-C₂-alkylsulfonyl-C₁-C₂-alkyl; C₁-C₂-alkylsulfonyl-cyclopropyl, C₁-C₂-cyanoalkyl, and cyano-cyclopropyl; as R⁴ phenyl substituted with 1 or 2 substituents independently selected from halogen, C₁-C₄-alkyl, C₁-C₄-haloalkyl, cyano, C₁-C₄-haloalkylsulfanyl, C₁-C₄-alkylsulfonyl and C₁-C₄-haloalkoxy, one of thiophenyl, pyridyl (or pyridinyl), and pyrazolyl—each substituted with 1 to 2 substituents independently selected from fluorine, chlorine, methyl, ethyl, isopropyl, trifluoromethyl, difluoromethyl, trifluoromethoxy, and difluoromethoxy; as Q cyclic amine represented by the formula IIa, where both p¹ and p² are 1, X is hydrogen or hydroxyl; and Y is cyano, C₁-C₄-alkylsulfanyl-C₁-C₄-alkyl, C₁-C₄-alkylsulfinyl-C₁-C₄-alkyl, C₁-C₄-alkylsulfonyl-C₁-C₄-alkyl, 1,1-dioxo-1,2-thiazolidin-2-yl, N,N-di(C₁-C₄-alkyl)carboxamide, C₁-C₄-alkoxy-imino-C₁-C₄-alkyl, C₁-C₄-alkylsulfonylamide, phenyl, phenyl substituted with 1 to 3 substituents independently selected from halogen, C₁-C₃-alkyl, and C₁-C₃-haloalkyl and C₁-C₃-haloalkoxy, or 5 or 6 membered monocyclic heteroaryl substituted with 1 to 3 substituents independently selected from halogen, C₁-C₃-alkyl, and C₁-C₃-haloalkyl and C₁-C₃-haloalkoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoromethyl, trifluoroethyl, trifluoromethoxy, trifluoromethylsulfanyl, cyano, and methylsulfonyl, or one of thiophenyl, pyridyl (or pyridinyl), and pyrazolyl—each substituted with 1 to 2 substituents independently selected from fluorine, chlorine, methyl, ethyl, isopropyl, trifluoromethyl, difluoromethyl, trifluoromethoxy, and difluoromethoxy; as Q cyclic amine represented by the formula IIa, where both p¹ and p² are 1, X is hydrogen or hydroxyl; and Y is cyano, C₁-C₂-alkylsulfanyl-C₁-C₂-alkyl, C₁-C₂-alkylsulfinyl-C₁-C₂-alkyl, C₁-C₂-alkylsulfonyl-C₁-C₂-alkyl, 1,1-dioxo-1,2-thiazolidin-2-yl, N,N-di(C₁-C₂-alkyl)carboxamide, C₁-C₂-alkoxy-imino-C₁-C₂-alkyl, C₁-C₂-alkylsulfonylamide, phenyl, phenyl substituted with 1 to 3 substituents independently selected from chloro, methyl, difluoromethyl, trifluoroethyl, trifluoromethyl, and trifluoromethoxy, or 5 or 6 membered monocyclic heteroaryl substituted with 1 to 3 substituents independently selected from chloro, fluoro, methyl, trifluoroethyl, trifluoromethyl and trifluoromethoxy.

In an embodiment of each aspect of the invention, the compound of formula (I) has as R¹ cyano, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoroethyl, trifluoromethyl, trifluoromethoxy, trifluoromethylsulfanyl, cyano, and methylsulfonyl or one of thiophenyl, pyridyl (or pyridinyl), and pyrazolyl—each substituted with 1 to 2 substituents independently selected from fluorine, chlorine, methyl, ethyl, isopropyl, trifluoromethyl, difluoromethyl, trifluoromethoxy, and difluoromethoxy; as Q cyclic amine represented by the formula IIa, where both p¹ and p² are 1, X is hydrogen or hydroxyl; and Y is cyano, C₁-C₂-alkylsulfanyl-C₁-C₂-alkyl, C₁-C₂-alkylsulfinyl-C₁-C₂-alkyl, C₁-C₂-alkylsulfonyl-C₁-C₂-alkyl, 1,1-dioxo-1,2-thiazolidin-2-yl, N,N-di(C₁-C₂-alkyl)carboxamide, C₁-C₂-alkoxy-imino-C₁-C₂-alkyl, C₁-C₂-alkylsulfonylamide, phenyl, phenyl substituted with 1 to 3 substituents independently selected from chloro, methyl, difluoromethyl, trifluoroethyl, trifluoromethyl, and trifluoromethoxy, or 5 or 6 membered monocyclic heteroaryl selected from 1,2,4-oxadiazol-3-yl, pyridine-2-yl and 1,2,4-triazole-3-yl, wherein each heteroaryl is substituted with 1 to 3 substituents independently selected from chloro, fluoro, methyl, trifluoroethyl, trifluoromethyl and trifluoromethoxy.

In an embodiment of each aspect of the invention, the compound of formula (I) has as R¹ cyano, as R² hydrogen, as R³ is methoxymethyl, ethoxymethyl, methysulfonylmethyl, methysulfonylcyclopropyl, cyano-methyl or cyano-cyclopropyl; as R⁴ phenyl substituted with 1 or 2 substituents independently selected from iodo, bromo, chloro, fluoro, methyl, trifluoromethyl, trifluoromethoxy, trifluoromethylsulfanyl, cyano, and methylsulfonyl, or one of thiophenyl, pyridyl (or pyridinyl), and pyrazolyl—each substituted with 1 to 3 substituents independently selected from fluorine, chlorine, methyl, ethyl, isopropyl, trifluoromethyl, difluoromethyl, trifluoromethoxy, and difluoromethoxy; as Q cyclic amine represented by the formula IIa, where both p¹ and p² are 1, X is hydrogen; and Y is cyano, C₁-C₂-alkylsulfanyl-C₁-C₂-alkyl, C₁-C₂-alkylsulfinyl-C₁-C₂-alkyl, C₁-C₂-alkylsulfonyl-C₁-C₂-alkyl, 1,1-dioxo-1,2-thiazolidin-2-yl, N,N-di(C₁-C₂-alkyl)carboxamide, C₁-C₂-alkoxy-imino-C₁-C₂-alkyl, C₁-C₂-alkylsulfonylamide, phenyl, phenyl substituted with 1 to 3 substituents independently selected from chloro, methyl, difluoromethyl, trifluormethyl, and trifluoromethoxy, or 5 or 6 membered monocyclic heteroaryl selected from 1,2,4-oxadiazol-3-yl, pyridine-2-yl and 1,2,4-triazole-3-yl, wherein each heteroaryl is substituted with 1 to 3 substituents independently selected from chloro, fluoro, methyl, trifluoromethyl and trifluoromethoxy.

In an embodiment of each aspect of the invention, the compound of formula (I) has as R¹ cyano, as R² hydrogen, as R³ is methoxymethyl; as R⁴ phenyl substituted with a substituent selected from chloro, fluoro, trifluoromethyl, as Q cyclic amine represented by the formula IIa, where both p¹ and p² are 1, X is hydrogen; and Y is 5 or 6 membered monocyclic heteroaryl selected from 1,2,4-oxadiazol-3-yl, pyridine-2-yl and 1,2,4-triazole-3-yl, wherein each heteroaryl is substituted with 1 to 3 substituents independently selected from chloro, fluoro, methyl, trifluoromethyl and trifluoromethoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₄-alkyl, C₁-C₄-haloalkyl, cyano, C₁-C₄-haloalkylsulfanyl, C₁-C₄-alkylsulfonyl and C₁-C₄-haloalkoxy, one of thiophenyl, pyridyl (or pyridinyl), and pyrazolyl—each substituted with 1 to 2 substituents independently selected from halogen, C₁-C₄-alkyl, C₁-C₄-haloalkyl, and C₁-C₄-haloalkoxy; as Q cyclic amine represented by the formula IIb, where both p¹ and p² are 1, A is cyano, C₁-C₄-cyanoalkyl, C₁-C₄-haloalkyl, C₁-C₄-alkylsulfanyl-C₁-C₄-alkyl, C₁-C₄-alkylsulfinyl-C₁-C₄-alkyl, C₁-C₄-alkylsulfonyl-C₁-C₄-alkyl, C₁-C₄-alkoxycarbonyl, C₂-C₄-alkenyloxycarbonyl, phenyl, phenyl substituted with 1 to 3 substituents independently selected from halogen, C₁-C₃-alkyl, and C₁-C₃-haloalkyl and C₁-C₃-haloalkoxy, or 5 or 6 membered monocyclic heteroaryl substituted with 1 to 3 substituents independently selected from halogen, C₁-C₃-alkyl, and C₁-C₃-haloalkyl and C₁-C₃-haloalkoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoromethyl, trifluoromethoxy, trifluoromethylsulfanyl, cyano, and methylsulfonyl; as Q cyclic amine represented by the formula IIb, where both p¹ and p² are 1, A is cyano, C₁-C₄-cyanoalkyl, C₁-C₄-haloalkyl, C₁-C₄-alkylsulfanyl-C₁-C₄-alkyl, C₁-C₄-alkylsulfinyl-C₁-C₄-alkyl, C₁-C₄-alkylsulfonyl-C₁-C₄-alkyl, C₁-C₄-alkoxycarbonyl, C₂-C₄-alkenyloxycarbonyl, phenyl, phenyl substituted with 1 to 3 substituents independently selected from halogen, C₁-C₃-alkyl, and C₁-C₃-haloalkyl and C₁-C₃-haloalkoxy, or 5 or 6 membered monocyclic heteroaryl substituted with 1 to 3 substituents independently selected from halogen, C₁-C₃-alkyl, and C₁-C₃-haloalkyl and C₁-C₃-haloalkoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoromethyl, trifluoromethoxy, trifluoromethylsulfanyl, cyano, and methylsulfonyl; as Q cyclic amine represented by the formula IIb, where both p¹ and p² are 1, A is cyano, C₁-C₄-cyanoalkyl, C₁-C₄-haloalkyl, C₁-C₄-alkylsulfanyl-C₁-C₄-alkyl, C₁-C₄-alkylsulfinyl-C₁-C₄-alkyl, C₁-C₄-alkylsulfonyl-C₁-C₄-alkyl, C₁-C₄-alkoxycarbonyl, C₂-C₄-alkenyloxycarbonyl, phenyl, phenyl substituted with 1 to 3 substituents independently selected from chloro, methyl, difluoromethyl, trifluormethyl, and trifluoromethoxy, or 5 or 6 membered monocyclic heteroaryl substituted with 1 to 3 substituents independently selected from chloro, methyl, difluoromethyl, trifluormethyl, and trifluoromethoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoromethyl, trifluoromethoxy, trifluoromethylsulfanyl, cyano, and methylsulfonyl; as Q cyclic amine represented by the formula IIb, where both p¹ and p² are 1, A is pyridyl substituted with 1 to 3 substituents independently selected from chloro, fluoro, methyl, difluoromethyl, trifluormethyl, and trifluoromethoxy.

In an embodiment of each aspect of the invention, the compound of formula (I) has as R¹ cyano or C(═S)NH₂, as R² hydrogen, as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from iodo, bromo, chloro, fluoro, methyl, trifluoromethyl, trifluoromethoxy, trifluoromethylsulfanyl, cyano, and methylsulfonyl; as Q cyclic amine represented by the formula IIb, where both p¹ and p² are 1, A is pyridyl substituted with 1 to 3 substituents independently selected from chloro, fluoro, trifluormethyl, and trifluoromethoxy.

In an embodiment of each aspect of the invention, the compound of formula (I) has as R¹ cyano; as R² hydrogen; as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-alkyl, C₁-C₃-haloalkyl, C₁-C₃-haloalkoxy and —O—C₁₋₂haloalkanediyl-O—; as Q cyclic amine represented by the formula IIa, where both p¹ and p² are 1, X is hydrogen; and Y is 5 or 6 membered monocyclic heteroaryl or 5 or 6 membered monocyclic heteroaryl substituted with 1 to 3 independently selected substituents R⁹.

In an embodiment of each aspect of the invention, the compound of formula (I) has as R¹ cyano; as R² hydrogen; as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-alkyl, C₁-C₃-haloalkyl, C₁-C₃-alkoxy, C₁-C₃-alkylsulfonyl, C₁-C₃-haloalkylsulfanyl, cyano, C₁-C₃-haloalkoxy and —O—C₁₋₂haloalkanediyl-O—; as Q cyclic amine represented by the formula IIa, where both p1 and p2 are 1, X is hydrogen; and Y is a oxadiazolyl substituted with 1 to 3 independently selected substituents R⁹.

In an embodiment of each aspect of the invention, the compound of formula (I) has as R¹ cyano; as R² hydrogen; as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-haloalkyl, C₁-C₃-haloalkoxy and —O—C₁₋₂haloalkanediyl-O—; as Q cyclic amine represented by the formula IIa, where both p1 and p2 are 1, X is hydrogen; and Y is a 1,2,4-oxadiazol-3-yl substituted with 1 to 2 substituents independently selected from methyl, ethyl, and isopropyl.

In an embodiment of each aspect of the invention, the compound of formula (I) has as R¹ cyano; as R² hydrogen; as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-haloalkyl, C₁-C₃-haloalkoxy and —O—C₁₋₂haloalkanediyl-O—; as Q cyclic amine represented by the formula IIb, where both q1 and q2; are 1 and A is a pyridyl substituted with 1 to 3 independently selected halogen substituents.

In an embodiment of each aspect of the invention, the compound of formula (I) has as R¹ cyano; as R² hydrogen; as R³ selected from methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-haloalkyl, C₁-C₃-haloalkoxy and —O—C₁-2haloalkanediyl-O—; as Q cyclic amine represented by the formula IIb, where both q1 and q2 are 1 and A is a pyridyl substituted with 1 to 2 independently selected substituents from chlorine, fluorine and bromine.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, C₁-C₃-haloalkyl, C₁-C₃-haloalkoxy; as Q cyclic amine represented by the formula IIa, where both p1 and p2 are 1, X is hydrogen; and Y is C₁-C₃-alkoxymethyl, C₁-C₃-alkoxyN═CH—, C₁-C₃-alkylsulfanylmethyl, C₁-C₃-alkylsulfinylmethyl, C₁-C₃-alkylsulfonylmethyl,-1,1-dioxo-thiazolidinyl, 1-methylpyrazolyl, C₁-C₃-alkylsulfonyl-C₁-C₃-alkylamino, methyl-1,2,4-oxadiazolyl, or methylisoxazoleyl.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, C₁-C₃-haloalkyl, C₁-C₃-haloalkoxy; and as Q cyclic amine represented by the formula IIa, where both p1 and p2 are 1, X is hydrogen; and Y is —CH₂SCH₂CH₃; —CH₂S(═O)CH₂CH₃, —CH₂SO₂CH₂CH₃, 1,1-dioxo-1,2-thiazolidin-2-yl, 1-methylpyrazol-4-yl, methyl(methylsulfonyl)-amino (i.e CH₃S(O)₂—(CH₃)N—, 3-methyl-1,2,4-oxadiazol-5-yl, 1-methylpyrazol-3-yl or 3-methylisoxazole-5-yl.

In an embodiment of each aspect of the invention, the compound of formula (I) has as R¹ cyano; as R² hydrogen; as R³ methoxymethyl; as R⁴ one of 2,1,3-benzoxadiazolyl, 1,3-benzoxazolyl, thiophenyl, pyridyl (or pyridinyl), and pyrazolyl—independent of each other, is unsubstituted or substituted with 1 to 3 substituents independently selected from halogen, C₁-C₄-alkyl, C₁-C₄-haloalkyl, and C₁-C₄-haloalkoxy; and as Q cyclic amine represented by the formula IIa, where both p1 and p2 are 1, X is hydrogen; and Y is a 1,2,4-oxadiazol-3-yl substituted with 1 to 2 substituents independently selected from methyl, ethyl, and isopropyl.

In an embodiment of each aspect of the invention, the compound of formula (I) has as R¹ cyano; as R² hydrogen; as R³ methoxymethyl; as R⁴ one of thiophenyl, pyridyl (or pyridinyl), and pyrazolyl—each substituted with 1 to 3 substituents independently selected from halogen, C₁-C₄-alkyl, C₁-C₄-haloalkyl, and C₁-C₄-haloalkoxy; and as Q cyclic amine represented by the formula IIa, where both p1 and p2 are 1, X is hydrogen; and Y is 5-methyl-1,2,4-oxadiazol-3-yl.

In an embodiment of each aspect of the invention, the compound of formula (I) has as R¹ cyano or C(═S)NH₂; as R² hydrogen; as R³ methoxymethyl; as R⁴ phenyl with 1 to 2 substituents independently selected from halogen and C₁-C₄-haloalkyl; and as Q cyclic amine represented by the formula IIa, where both p1 and p2 are 1, X is hydrogen; and Y is a 1,2,4-oxadiazol-3-yl substituted with 1 to 2 substituents independently selected from methyl, ethyl, and isopropyl.

In an embodiment of each aspect of the invention, the compound of formula (I) has as R¹ cyano or C(═S)NH₂; as R² hydrogen; as R³ methoxymethyl; as R⁴ phenyl with 1 to 2 substituents independently selected from fluorine and trifluoromethyl; and as Q cyclic amine represented by the formula IIa, where both p1 and p2 are 1, X is hydrogen; and Y is 5-methyl-1,2,4-oxadiazol-3-yl.

In an embodiment of each aspect of the invention, the compound of formula (I) has as R¹ cyano; as R² hydrogen; as R³ methoxymethyl; as R⁴ one of phenyl, thiophenyl, pyridyl (or pyridinyl), and pyrazolyl—each substituted with 1 to 3 substituents independently selected from halogen, C₁-C₄-alkyl, C₁-C₄-haloalkyl, C₁-C₄-haloalkoxy and —O—C₁₋₂haloalkanediyl-O—; and as Q cyclic amine represented by the formula IIa, where both p1 and p2 are 1, X is hydrogen; and Y is a C₁-C₃-alkyl substituted 1,2,4-triazolyl.

In an embodiment of each aspect of the invention, the compound of formula (I) has as R¹ cyano; as R² hydrogen; as R³ methoxymethyl; as R⁴ one of phenyl, thiophenyl, pyridyl (or pyridinyl), and pyrazolyl—each substituted with 1 to 3 substituents independently selected from halogen, C₁-C₄-alkyl, C₁-C₄-haloalkyl, C₁-C₄-haloalkoxy and —O—C₁₋₂haloalkanediyl-O—; and as Q cyclic amine represented by the formula IIa, where both p1 and p2 are 1, X is hydrogen; and Y is 1-methyl-1,2,4-triazol-3-yl.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, and C₁-C₃-haloalkyl; and as Q cyclic amine represented by the formula IIb, where both q1 and q2 are 1 and A is C₁-C₃-alkylamino-sulfonyl, di(C₁-C₃-alkyl)amino-sulfonyl, pyrazol-3-yl, or oxadiazolyl.

In an embodiment of each aspect of the invention, the compound of formula (I) has as R¹ cyano; as R² hydrogen; as R³ methoxymethyl; as R⁴ phenyl substituted with 1 or 2 substituents independently selected from fluorine and trifluoromethyl; and as Q cyclic amine represented by the formula IIb, where both q1 and q2 are 1 and A is methylamino-sulfonyl, dimethylamino-sulfonyl, 1-methylpyrazol-3-yl, 5-methyl-1,2,4-oxadiazol-3-yl or 3-methyl-1,2,4-oxadiazol-5-yl.

In an embodiment of each aspect of the invention, the compound of formula (I) has as R¹ cyano; as R² hydrogen; as R³ methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-haloalkyl, C₁-C₃-haloalkoxy and —O—C₁₋₂haloalkanediyl-O—; and as Q cyclic amine represented by the formula IIa, where both q1 and q2 are 1, X is hydrogen, and Y is methoxymethyl, ethylsulfanylmethyl, ethylsulfinylmethyl, ethylsulfonylmethyl, 1-methyl-1,2,4-triazole-3-yl, 1,1-dioxo-1,2-thiazolidin-2-yl, 1-methylpyrazol-3-yl, 5-methyl-1,1-dioxo-1,2,5-thiadiazolidine-2-yl, or 5-ethyl-1,1-dioxo-1,2,5-thiadiazolidine-2-yl.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, C₁-C₃-haloalkyl, C₁-C₃-haloalkoxy and —O—C₁₋₂haloalkanediyl-O—; and as Q cyclic amine represented by the formula IIa, where both q1 and q2 are 1, X is hydrogen, and Y is methoxymethyl, ethylsulfanylmethyl, ethylsulfinylmethyl, ethylsulfonylmethyl, 1-methyl-1,2,4-triazole-3-yl, 1,1-dioxo-1,2-thiazolidin-2-yl, 1-methylpyrazol-3-yl, 5-methyl-1,1-dioxo-1,2,5-thiadiazolidine-2-yl, or 5-ethyl-1,1-dioxo-1,2,5-thiadiazolidine-2-yl.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, C₁-C₃-haloalkyl, C₁-C₃-haloalkoxy and —O—C₁₋₂haloalkanediyl-O—; and as Q cyclic amine represented by the formula IIa, where both q1 and q2 are 1, X is hydrogen, and Y is 1,1-dioxo-1,2-thiazolidin-2-yl, 5-methyl-1,1-dioxo-1,2,5-thiadiazolidine-2-yl, or 5-ethyl-1, 1-dioxo-1,2,5-thiadiazolidine-2-yl.

In an embodiment of each aspect of the invention, the compound of formula (I) has as R¹ cyano; as R² hydrogen; as R³ methoxymethyl, ethoxymethyl, methysulfonylmethyl, methysulfonylcyclopropyl, cyano-methyl and cyano-cyclopropyl; as R⁴ phenyl substituted with 1 or 2 substituents independently selected from halogen, C₁-C₃-haloalkyl, C₁-C₃-haloalkoxy and —O—C₁₋₂haloalkanediyl-O—; and as Q cyclic amine represented by the formula IIb, where both q1 and q2 are 1 and A is ethylsulfonyl, propylsulfonyl, iso-propylsulfonyl, cyclopropylsulfonyl, methylsulfamoyl, ethylsulfamoyl, dimethylsulfamoyl, ethyl(methyl)sulfamoyl, 4-fluoro-phenyl, 3,5-dichloropyridine-2-yl, 4-fluoro-pyridine-2-yl, 5-methyl-1,2,4-oxadiazol-3-yl, 3-methyl-1,2,4-oxadiazol-5-yl, or 1-methylpyrazol-3-yl.

In an embodiment of each aspect of the invention, the compound of formula (I) has as R¹ cyano; as R² hydrogen; as R³ methoxymethyl; as R⁴ phenyl substituted with 1 or 2 substituents independently selected from halogen, C₁-C₃-haloalkyl, C₁-C₃-haloalkoxy and —O—C₁₋₂haloalkanediyl-O—; and as Q cyclic amine represented by the formula IIb, where both q1 and q2 are 1 and A is ethylsulfonyl, propylsulfonyl, iso-propylsulfonyl, cyclopropylsulfonyl, methylsulfamoyl, ethylsulfamoyl, dimethylsulfamoyl, ethyl(methyl)sulfamoyl, 4-fluoro-phenyl, 3,5-dichloropyridine-2-yl, 4-fluoro-pyridine-2-yl, 5-methyl-1,2,4-oxadiazol-3-yl, 3-methyl-1,2,4-oxadiazol-5-yl, or 1-methylpyrazol-3-yl.

In an embodiment of each aspect of the invention, the compound of formula (I) has as R¹ cyano; as R² hydrogen; as R³ methoxymethyl; as R⁴ phenyl substituted with 1 to 2 substituents independently selected from halogen, and C₁-C₄-haloalkyl; and as Q cyclic amine represented by the formula IIa, where both p1 and p2 are 1, X is hydroxyl; and Y is 1 to 2 halogen disubstituted phenyl.

In an embodiment of each aspect of the invention, the compound of formula (I) has as R¹ cyano; as R² hydrogen; as R³ methoxymethyl; as R⁴ phenyl substituted with 1 to 2 substituents independently selected from fluorine and trifluoromethyl; and as Q cyclic amine represented by the formula IIa, where both p1 and p2 are 1, X is hydroxyl; and Y is chloro substituted phenyl.

In an embodiment of each aspect of the invention, the compound of formula (I) has as R¹ cyano; as R² hydrogen; as R³ methoxymethyl; as R⁴ phenyl substituted with 1 to 2 substituents independently selected from halogen, and C₁-C₄-haloalkyl; and as Q cyclic amine represented by the formula IIa, where both p1 and p2 are zero, X is hydrogen; and Y is C₁-C₃-alkylsulfanylmethyl, C₁-C₃-alkylsulfonylmethyl or oxadiazolyl.

In an embodiment of each aspect of the invention, the compound of formula (I) has as R¹ cyano; as R² hydrogen; as R³ methoxymethyl; as R⁴ trifluoromethyl substituted phenyl; and as Q cyclic amine represented by the formula IIa, where both p1 and p2 are zero, X is hydrogen; and Y is n-propylsulfonylmethyl.

In a second aspect, the present invention makes available a composition comprising a compound of formula (I) as defined in the first aspect, one or more auxiliaries and diluent, and optionally one more other active ingredient.

In a third aspect, the present invention makes available a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in the first aspect or a composition as defined in the second aspect.

In a fourth aspect, the present invention makes available a method for the protection of plant propagation material from the attack by insects, acarines, nematodes or molluscs, which comprises treating the propagation material or the site, where the propagation material is planted, with an effective amount of a compound of formula (I) as defined in the first aspect or a composition as defined in the second aspect.

In a fifth aspect, the present invention makes available a plant propagation material, such as a seed, comprising, or treated with or adhered thereto, a compound of formula (I) as defined in the first aspect or a composition as defined in the second aspect.

The present invention in a further aspect provides a method of controlling parasites in or on an animal in need thereof comprising administering an effective amount of a compound of the first aspect. The present invention further provides a method of controlling ectoparasites on an animal in need thereof comprising administering an effective amount of a compound of formula (I) as defined om the first aspect. The present invention further provides a method for preventing and/or treating diseases transmitted by ectoparasites comprising administering an effective amount of a compound of formula (I) as defined in the first aspect, to an animal in need thereof.

Compounds of formula (I) can be prepared by those skilled in the art following known methods. More specifically compounds of formulae I, and I′a, and intermediates therefor can be prepared as described below in the schemes and examples. Certain stereogenic centers have been left unspecified for the clarity and are not intended to limit the teaching of the schemes in any way.

The process according to the invention for preparing compounds of formula (I) is carried out by methods known to those skilled in the art.

The compounds of formula (I) are new and can be prepared by reacting an acid III in which R¹, R², R³, and R⁴ are as previously defined with an amine IV-a or IV-b in which X, Y, A, p¹, p², q¹ and q² are as previously defined using known amide coupling reagents, such as 1-[bis(dimethylamino)-methylene]-1H-1,2,3-triazolo[4,5-b]pyridinium 3-oxide hexafluorophosphate (HATU) and a base, for example Hunig's base, in a suitable solvent, for example dimethylformamide (DMF) or dimethylacetamide (DMA) according to scheme 1. Piperidines IV-a or piperazines IV-b are commercially available, known from the literature or can be prepared by the person skilled in the art.

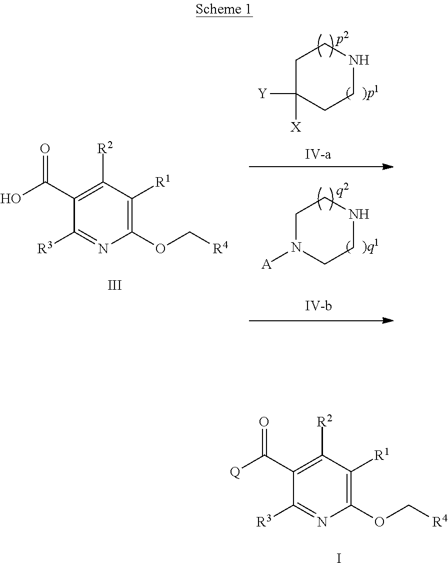

Alternatively the compounds of formula (I) can be prepared by reacting an acid chloride V in which R¹, R², R³ and R⁴ are as previously defined with an amine IV-a or IV-b in which X, Y, A, p¹, p², q¹ and q² are as previously defined in the presence of a base, for example triethylamine or pyridine, and a suitable solvent, for example dichloromethane (DCM), tetrahydrofuran (THF) or toluene, according to scheme 2.

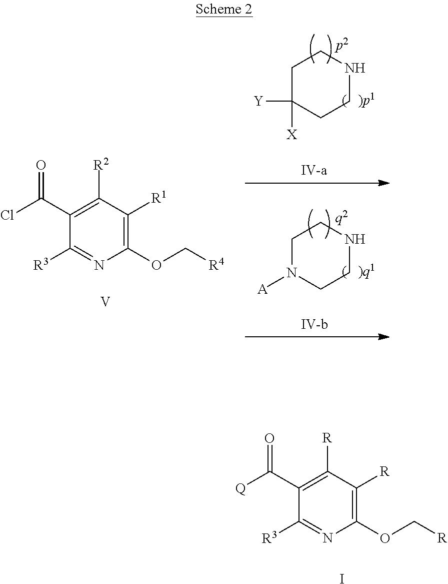

The acid chloride V in which R¹, R², R³ and R⁴ are as previously defined can be prepared from the corresponding acid III in which R¹, R², R³ and R⁴ are as previously defined by treatment with for example, oxalyl chloride or thionyl chloride in the presence of catalytic quantities of DMF in inert solvents such as DCM or THE at temperatures between 20° C. to 100° C., preferably 25° C. according to scheme 3.

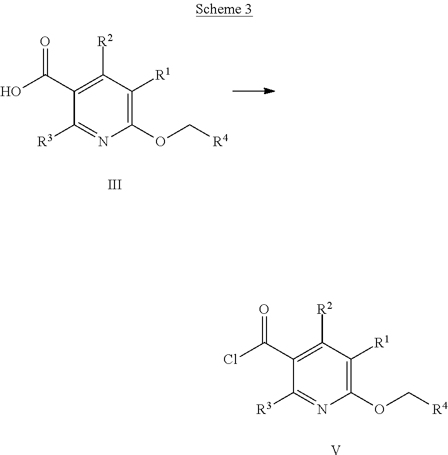

The acid III in which R¹, R², R³ and R⁴ are as previously defined can be prepared by hydrolysis of the corresponding ester VI in which in which R¹, R², R³ and R⁴ are as previously defined and R^{x}=C₁-C₆-alkyl under basic conditions, for example using an inorganic base such as lithium hydroxide, sodium hydroxide, potassium hydroxide or potassium carbonate in water, methanol, ethanol or THF, according to scheme 4.

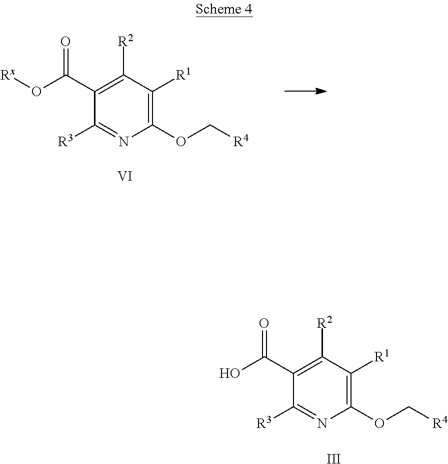

The ester VI in which R¹, R², R³ and R⁴ are as previously defined can be prepared by reaction of the pyridone VII in which R¹, R², R³, R⁴ and R^{x} are as previously defined with an alkylating reagent VIII in which R⁴ is as previously defined and LG¹ is a leaving group, such as a halogen, e.g. bromine, chlorine, iodine, or a mesylate in the presence of a base, for example an inorganic base such as lithium hydroxide, sodium hydroxide, potassium hydroxide or potassium carbonate, and a suitable solvent, for example water, methanol, ethanol, acetone, THF, DMF or toluene according to scheme 5. It might be advantageous to add sodium iodide or a phase-transfer catalyst, for example tetrabutylammonium bromide or tetrabutylammonium iodide.

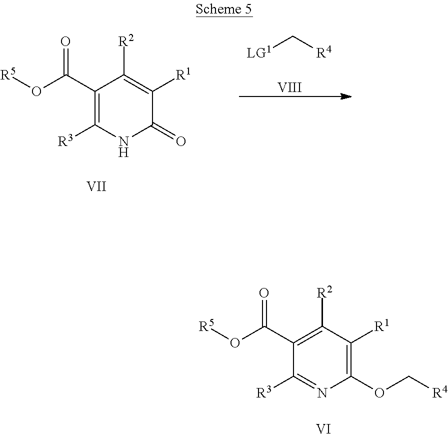

Alternatively, the ester VI can be prepared by reacting the pyridine IX in which R¹, R², R³, R^{x} are as previously defined and LG² is a leaving group, such as a halogen, e.g. fluorine, bromine, chlorine, iodine, or a mesylate with an alcohol X in the presence of a base, for example sodium hydride, lithium hydroxide, sodium hydroxide, potassium hydroxide or potassium carbonate and a suitable solvent, for example THF, DMF or toluene, according to scheme 6.

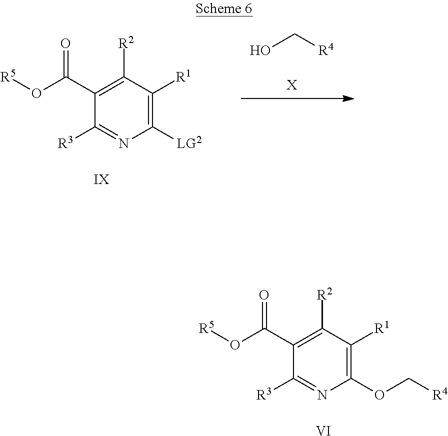

Alternatively, the compounds of formula (I) can be obtained by reaction of the pyridone XI in which R¹, R², R³ and Q are as previously defined with an alkylating reagent VIII in which R⁴ is as previously defined and LG¹ is a leaving group, such as a halogen, e.g. bromine, chlorine, iodine, or a mesylate in the presence of a base, for example an inorganic base such as lithium hydroxide, sodium hydroxide, potassium hydroxide or potassium carbonate, in a suitable solvent, for example water, methanol, ethanol, acetone, THF, DMF or toluene a according to scheme 7. It might be advantageous to add sodium iodide or a phase-transfer catalyst, for example tetrabutylammonium bromide or tetrabutylammonium iodide.

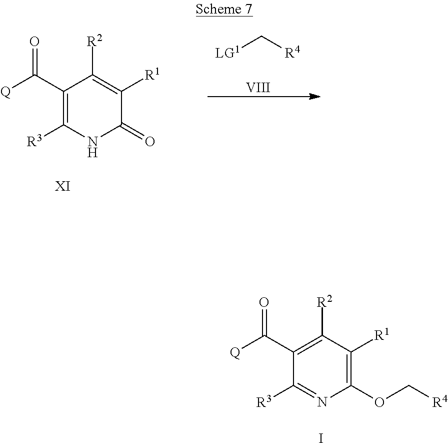

Alternatively, the compounds of formula (I) can be prepared by reacting the pyridine XII in which R¹, R², R³ and Q are as previously defined and LG² is a leaving group, such as a halogen, e.g. fluorine, bromine, chlorine, iodine, or a mesylate with an alcohol X in the presence of a base, for example sodium hydride, lithium hydroxide, sodium hydroxide, potassium hydroxide or potassium carbonate and a suitable solvent, for example THF, DMF or toluene according to scheme 8.

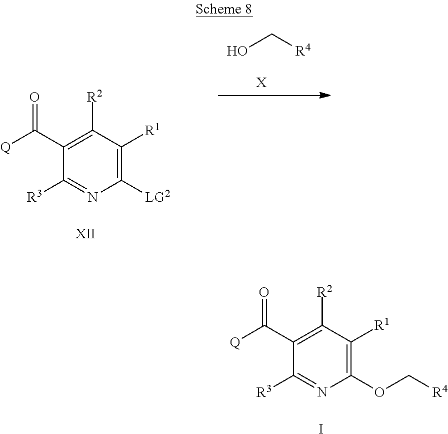

Compounds of formula XII in which R¹, R², R³ and Q are as previously defined and LG² is a leaving group can be obtained by treating a pyridone XI in which R¹, R², R³ and Q are as previously defined with a halogenation reagent, such as phosphorus oxychloride or oxalyl chloride or mesylchloride according to scheme 9.

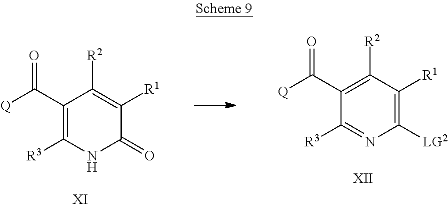

Pyridones XI in which R¹, R², R³ and Q are as previously defined can be prepared by reacting an acid XIII in which R¹, R² and R³ are as previously defined with an amine IV-a or IV-b in which X, Y, A, p¹, p², q¹ and q² are as previously defined using known amide coupling reagents, such as HATU and a base, for example Hunig's base, in a suitable solvent, for example DMF or DMA according to scheme 10.

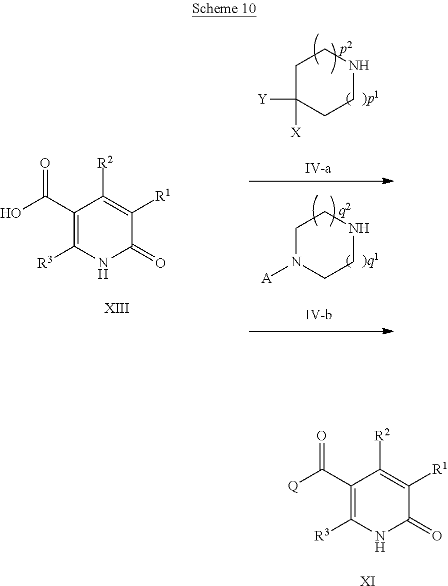

Alternatively the compounds of formula XI in which R¹, R², R³ and Q are as previously defined can be prepared by reacting an acid chloride XIV in which R¹, R² and R³ are as previously defined with an amine IV-a or IV-b in the presence of a base, for example triethylamine or pyridine, and a suitable solvent, for example DCM, THE or toluene, according to scheme 11.

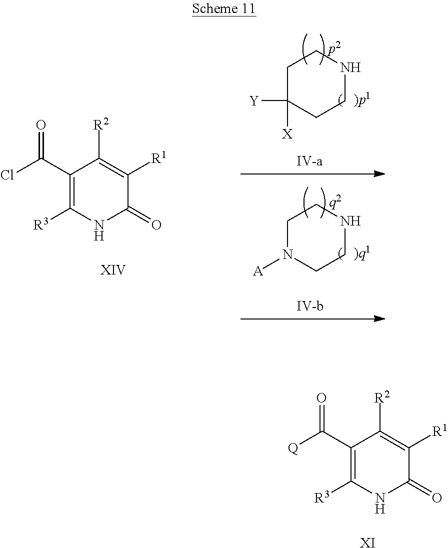

The acid chloride XIV in which R¹, R² and R³ are as previously defined can be prepared from the corresponding acid XIII in which R¹, R² and R³ are as previously defined by treatment with for example, oxalyl chloride or thionyl chloride in the presence of catalytic quantities of DMF in inert solvents such as DCM or THE at temperatures between 20° C. to 100° C., preferably 25° C. according to scheme 12.

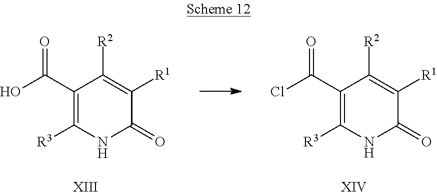

The acid XIII in which R¹, R² and R³ are as previously defined can be prepared by hydrolysis of the corresponding ester XV in which R¹, R², R³ and R^{x} are as previously defined under basic conditions, for example using an inorganic base such as lithium hydroxide, sodium hydroxide, potassium hydroxide or potassium carbonate in water, methanol, ethanol or THF. Esters of the formula XV in which R¹, R², R³ and R^{x} are as previously defined are known in the literature, for example Y. Xie et al., Pest Manag. Sci. 2017, 73, 945-952, or can be prepared by the person skilled in the art.

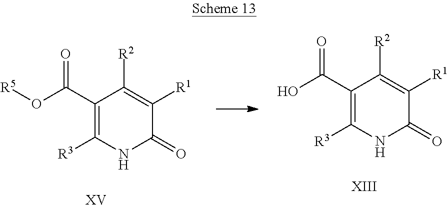

The compounds of formula (I) according to the following Tables 1 to 81 can be prepared according to the methods described above. The examples which follow are intended to illustrate the invention and show preferred compounds of formula (I), in the form of a compound of formula (I-a).

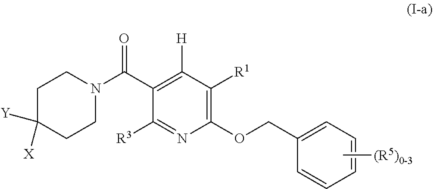

Each of Tables 1 to 48, which follow the Table M below, consist of 1316 compounds of the formula (I-a) in which R¹, R³ and R⁵ have the values given in each row in Table M, and Y and X have the values given in the relevant Tables 1 to 48.

Thus compound 1.1 corresponds to a compound of formula (I-a) where R¹, R³ and R⁵ are as defined in row 1 of Table M and where Y and X are as defined in Table 1; compound 14.14 corresponds to a compound of formula (I-a) where R¹, R³ and R⁵ are as defined in row 14 of Table M and where Y and X are as defined in Table 14; and so on.

TABLE M

| Comp. No | R¹ | R³ | R⁵ |
|---|---|---|---|
| M.1. | CN | CH₂OCH₃ | — |
| M.2. | CN | CH₂OCH₃ | 2-F |
| M.3. | CN | CH₂OCH₃ | 3-F |
| M.4. | CN | CH₂OCH₃ | 4-F |
| M.5. | CN | CH₂OCH₃ | 2-Cl |
| M.6. | CN | CH₂OCH₃ | 3-Cl |
| M.7. | CN | CH₂OCH₃ | 4-Cl |
| M.8. | CN | CH₂OCH₃ | 2-Br |
| M.9. | CN | CH₂OCH₃ | 3-Br |
| M.10. | CN | CH₂OCH₃ | 4-Br |
| M.11. | CN | CH₂OCH₃ | 4-I |
| M.12. | CN | CH₂OCH₃ | 2-CH₃ |
| M.13. | CN | CH₂OCH₃ | 3-CH₃ |
| M.14. | CN | CH₂OCH₃ | 4-CH₃ |
| M.15. | CN | CH₂OCH₃ | 2-CF₃ |
| M.16. | CN | CH₂OCH₃ | 3-CF₃ |
| M.17. | CN | CH₂OCH₃ | 4-CF₃ |
| M.18. | CN | CH₂OCH₃ | 2-OCF₃ |
| M.19. | CN | CH₂OCH₃ | 3-OCF₃ |
| M.20. | CN | CH₂OCH₃ | 4-OCF₃ |
| M.21. | CN | CH₂OCH₃ | 4-OCHF₂ |
| M.22. | CN | CH₂OCH₃ | 2-OCF₂O-3 |
| M.23. | CN | CH₂OCH₃ | 3-OCF₂O-4 |
| M.24. | CN | CH₂OCH₃ | 2-Cl, 4-O—CF₂—O-5 |
| M.25. | CN | CH₂OCH₃ | 2-SCF₃ |
| M.26. | CN | CH₂OCH₃ | 3-SCF₃ |
| M.27. | CN | CH₂OCH₃ | 4-SCF₃ |
| M.28. | CN | CH₂OCH₃ | 2-CN |
| M.29. | CN | CH₂OCH₃ | 3-CN |
| M.30. | CN | CH₂OCH₃ | 4-CN |
| M.31. | CN | CH₂OCH₃ | 3-CN, 4-F |
| M.32. | CN | CH₂OCH₃ | 3-CN, 4-Cl |
| M.33. | CN | CH₂OCH₃ | 2-F, 5-CN |
| M.34. | CN | CH₂OCH₃ | 2-Cl, 5-CN |
| M.35. | CN | CH₂OCH₃ | 2-F, 4-CN |
| M.36. | CN | CH₂OCH₃ | 2-Cl, 4-CN |
| M.37. | CN | CH₂OCH₃ | 3-F, 4-CN |
| M.38. | CN | CH₂OCH₃ | 3-Cl, 4-CN |
| M.39. | CN | CH₂OCH₃ | 3-SO₂CH₃ |
| M.40. | CN | CH₂OCH₃ | 4-SO₂CH₃ |
| M.41. | CN | CH₂OCH₃ | 2-F, 4-SO₂CH₃ |
| M.42. | CN | CH₂OCH₃ | 2-Cl, 4-SO₂CH₃ |
| M.43. | CN | CH₂OCH₃ | 3-F, 4-SO₂CH₃ |
| M.44. | CN | CH₂OCH₃ | 3-Cl, 4-SO₂CH₃ |
| M.45. | CN | CH₂OCH₃ | 3-SO₂CH₃, 4-F |
| M.46. | CN | CH₂OCH₃ | 3-SO₂CH₃, 4-Cl |
| M.47. | CN | CH₂OCH₃ | 2-F, 5-SO₂CH₃ |
| M.48. | CN | CH₂OCH₃ | 2-Cl, 5-SO₂CH₃ |
| M.49. | CN | CH₂OCH₃ | 2,3-F₂ |
| M.50. | CN | CH₂OCH₃ | 2,4-F₂ |
| M.51. | CN | CH₂OCH₃ | 2,5-F₂ |
| M.52. | CN | CH₂OCH₃ | 2,6-F₂ |
| M.53. | CN | CH₂OCH₃ | 3,4-F₂ |
| M.54. | CN | CH₂OCH₃ | 3,5-F₂ |
| M.55. | CN | CH₂OCH₃ | 2,4-Cl₂ |
| M.56. | CN | CH₂OCH₃ | 2,5-Cl₂ |
| M.57. | CN | CH₂OCH₃ | 3,4-Cl₂ |
| M.58. | CN | CH₂OCH₃ | 2,3,4-F₃ |
| M.59. | CN | CH₂OCH₃ | 2,3,5-F₃ |
| M.60. | CN | CH₂OCH₃ | 2,3,6-F₃ |
| M.61. | CN | CH₂OCH₃ | 3,4,5-F₃ |
| M.62. | CN | CH₂OCH₃ | 2,4,6-F₃ |
| M.63. | CN | CH₂OCH₃ | 2-F, 3-Cl |
| M.64. | CN | CH₂OCH₃ | 2-F, 4-Cl |
| M.65. | CN | CH₂OCH₃ | 2-F, 5-Cl |
| M.66. | CN | CH₂OCH₃ | 3-F, 4-Cl |
| M.67. | CN | CH₂OCH₃ | 2-Cl, 4-F |
| M.68. | CN | CH₂OCH₃ | 3-Cl, 4-F |
| M.69. | CN | CH₂OCH₃ | 2-Cl, 4,5-F₂ |
| M.70. | CN | CH₂OCH₃ | 2-Cl, 4-Br |
| M.71. | CN | CH₂OCH₃ | 2-Cl, 5-Br |
| M.72. | CN | CH₂OCH₃ | 3-Cl, 4-Br |
| M.73. | CN | CH₂OCH₃ | 2-F, 4-Br |
| M.74. | CN | CH₂OCH₃ | 2-F, 5-Br |
| M.75. | CN | CH₂OCH₃ | 3-F, 4-Br |
| M.76. | CN | CH₂OCH₃ | 2-Br, 4-F |
| M.77. | CN | CH₂OCH₃ | 3-Br, 4-F |
| M.78. | CN | CH₂OCH₃ | 2-Br, 5-Cl |
| M.79. | CN | CH₂OCH₃ | 3-Br, 4-Cl |
| M.80. | CN | CH₂OCH₃ | 2-Br, 4,5-F₂ |
| M.81. | CN | CH₂OCH₃ | 3- ═N—O—N═ -4 |
| M.82. | CN | CH₂OCH₃ | 4-CHF₂ |
| M.83. | CN | CH₂OCH₃ | 2-F, 3-CF₃ |
| M.84. | CN | CH₂OCH₃ | 2-F, 4-CF₃ |
| M.85. | CN | CH₂OCH₃ | 2-F, 5-CF₃ |
| M.86. | CN | CH₂OCH₃ | 3-F, 4-CF₃ |
| M.87. | CN | CH₂OCH₃ | 3-F, 5-CF₃ |
| M.88. | CN | CH₂OCH₃ | 4-F, 3-CF₃ |
| M.89. | CN | CH₂OCH₃ | 3,4-F₂, 5-CF₃ |
| M.90. | CN | CH₂OCH₃ | 2-Cl, 3-CF₃ |
| M.91. | CN | CH₂OCH₃ | 2-Cl, 4-CF₃ |
| M.92. | CN | CH₂OCH₃ | 2-Cl, 5-CF₃ |
| M.93. | CN | CH₂OCH₃ | 3-Cl, 5-CF₃ |
| M.94. | CN | CH₂OCH₃ | 4-Cl, 3-CF₃ |
| M.95. | CN | CH₂OCH₂CH₃ | — |
| M.96. | CN | CH₂OCH₂CH₃ | 2-F |
| M.97. | CN | CH₂OCH₂CH₃ | 3-F |
| M.98. | CN | CH₂OCH₂CH₃ | 4-F |
| M.99. | CN | CH₂OCH₂CH₃ | 2-Cl |
| M.100. | CN | CH₂OCH₂CH₃ | 3-Cl |
| M.101. | CN | CH₂OCH₂CH₃ | 4-Cl |
| M.102. | CN | CH₂OCH₂CH₃ | 2-Br |
| M.103. | CN | CH₂OCH₂CH₃ | 3-Br |
| M.104. | CN | CH₂OCH₂CH₃ | 4-Br |
| M.105. | CN | CH₂OCH₂CH₃ | 4-I |
| M.106. | CN | CH₂OCH₂CH₃ | 2-CH₃ |
| M.107. | CN | CH₂OCH₂CH₃ | 3-CH₃ |
| M.108. | CN | CH₂OCH₂CH₃ | 4-CH₃ |
| M.109. | CN | CH₂OCH₂CH₃ | 2-CF₃ |
| M.110. | CN | CH₂OCH₂CH₃ | 3-CF₃ |
| M.111. | CN | CH₂OCH₂CH₃ | 4-CF₃ |
| M.112. | CN | CH₂OCH₂CH₃ | 2-OCF₃ |
| M.113. | CN | CH₂OCH₂CH₃ | 3-OCF₃ |
| M.114. | CN | CH₂OCH₂CH₃ | 4-OCF₃ |
| M.115. | CN | CH₂OCH₂CH₃ | 4-OCHF₂ |
| M.116. | CN | CH₂OCH₂CH₃ | 2-OCF₂O-3 |
| M.117. | CN | CH₂OCH₂CH₃ | 3-OCF₂O-4 |
| M.118. | CN | CH₂OCH₂CH₃ | 2-Cl, 4-O—CF₂—O-5 |
| M.119. | CN | CH₂OCH₂CH₃ | 2-SCF₃ |
| M.120. | CN | CH₂OCH₂CH₃ | 3-SCF₃ |
| M.121. | CN | CH₂OCH₂CH₃ | 4-SCF₃ |
| M.122. | CN | CH₂OCH₂CH₃ | 2-CN |
| M.123. | CN | CH₂OCH₂CH₃ | 3-CN |
| M.124. | CN | CH₂OCH₂CH₃ | 4-CN |
| M.125. | CN | CH₂OCH₂CH₃ | 3-CN, 4-F |
| M.126. | CN | CH₂OCH₂CH₃ | 3-CN, 4-Cl |
| M.127. | CN | CH₂OCH₂CH₃ | 2-F, 5-CN |
| M.128. | CN | CH₂OCH₂CH₃ | 2-Cl, 5-CN |
| M.129. | CN | CH₂OCH₂CH₃ | 2-F, 4-CN |
| M.130. | CN | CH₂OCH₂CH₃ | 2-Cl, 4-CN |
| M.131. | CN | CH₂OCH₂CH₃ | 3-F, 4-CN |
| M.132. | CN | CH₂OCH₂CH₃ | 3-Cl, 4-CN |
| M.133. | CN | CH₂OCH₂CH₃ | 3-SO₂CH₃ |
| M.134. | CN | CH₂OCH₂CH₃ | 4-SO₂CH₃ |
| M.135. | CN | CH₂OCH₂CH₃ | 2-F, 4-SO₂CH₃ |
| M.136. | CN | CH₂OCH₂CH₃ | 2-Cl, 4-SO₂CH₃ |
| M.137. | CN | CH₂OCH₂CH₃ | 3-F, 4-SO₂CH₃ |
| M.138. | CN | CH₂OCH₂CH₃ | 3-Cl, 4-SO₂CH₃ |
| M.139. | CN | CH₂OCH₂CH₃ | 3-SO₂CH₃, 4-F |
| M.140. | CN | CH₂OCH₂CH₃ | 3-SO₂CH₃, 4-Cl |
| M.141. | CN | CH₂OCH₂CH₃ | 2-F, 5-SO₂CH₃ |
| M.142. | CN | CH₂OCH₂CH₃ | 2-Cl, 5-SO₂CH₃ |
| M.143. | CN | CH₂OCH₂CH₃ | 2,3-F₂ |
| M.144. | CN | CH₂OCH₂CH₃ | 2,4-F₂ |
| M.145. | CN | CH₂OCH₂CH₃ | 2,5-F₂ |
| M.146. | CN | CH₂OCH₂CH₃ | 2,6-F₂ |
| M.147. | CN | CH₂OCH₂CH₃ | 3,4-F₂ |
| M.148. | CN | CH₂OCH₂CH₃ | 3,5-F₂ |
| M.149. | CN | CH₂OCH₂CH₃ | 2,4-Cl₂ |
| M.150. | CN | CH₂OCH₂CH₃ | 2,5-Cl₂ |
| M.151. | CN | CH₂OCH₂CH₃ | 3,4-Cl₂ |
| M.152. | CN | CH₂OCH₂CH₃ | 2,3,4-F₃ |
| M.153. | CN | CH₂OCH₂CH₃ | 2,3,5-F₃ |
| M.154. | CN | CH₂OCH₂CH₃ | 2,3,6-F₃ |
| M.155. | CN | CH₂OCH₂CH₃ | 3,4,5-F₃ |
| M.156. | CN | CH₂OCH₂CH₃ | 2,4,6-F₃ |
| M.157. | CN | CH₂OCH₂CH₃ | 2-F, 3-Cl |
| M.158. | CN | CH₂OCH₂CH₃ | 2-F, 4-Cl |
| M.159. | CN | CH₂OCH₂CH₃ | 2-F, 5-Cl |
| M.160. | CN | CH₂OCH₂CH₃ | 3-F, 4-Cl |
| M.161. | CN | CH₂OCH₂CH₃ | 2-Cl, 4-F |
| M.162. | CN | CH₂OCH₂CH₃ | 3-Cl, 4-F |
| M.163. | CN | CH₂OCH₂CH₃ | 2-Cl, 4,5-F₂ |
| M.164. | CN | CH₂OCH₂CH₃ | 2-Cl, 4-Br |
| M.165. | CN | CH₂OCH₂CH₃ | 2-Cl, 5-Br |
| M.166. | CN | CH₂OCH₂CH₃ | 3-Cl, 4-Br |
| M.167. | CN | CH₂OCH₂CH₃ | 2-F, 4-Br |
| M.168. | CN | CH₂OCH₂CH₃ | 2-F, 5-Br |
| M.169. | CN | CH₂OCH₂CH₃ | 3-F, 4-Br |
| M.170. | CN | CH₂OCH₂CH₃ | 2-Br, 4-F |
| M.171. | CN | CH₂OCH₂CH₃ | 3-Br, 4-F |
| M.172. | CN | CH₂OCH₂CH₃ | 2-Br, 5-Cl |
| M.173. | CN | CH₂OCH₂CH₃ | 3-Br, 4-Cl |
| M.174. | CN | CH₂OCH₂CH₃ | 2-Br, 4,5-F₂ |
| M.175. | CN | CH₂OCH₂CH₃ | 3- ═N—O—N═ -4 |
| M.176. | CN | CH₂OCH₂CH₃ | 4-CHF₂ |
| M.177. | CN | CH₂OCH₂CH₃ | 2-F, 3-CF₃ |
| M.178. | CN | CH₂OCH₂CH₃ | 2-F, 4-CF₃ |
| M.179. | CN | CH₂OCH₂CH₃ | 2-F, 5-CF₃ |
| M.180. | CN | CH₂OCH₂CH₃ | 3-F, 4-CF₃ |
| M.181. | CN | CH₂OCH₂CH₃ | 3-F, 5-CF₃ |
| M.182. | CN | CH₂OCH₂CH₃ | 4-F, 3-CF₃ |
| M.183. | CN | CH₂OCH₂CH₃ | 3,4-F₂, 5-CF₃ |
| M.184. | CN | CH₂OCH₂CH₃ | 2-Cl, 3-CF₃ |
| M.185. | CN | CH₂OCH₂CH₃ | 2-Cl, 4-CF₃ |
| M.186. | CN | CH₂OCH₂CH₃ | 2-Cl, 5-CF₃ |
| M.187. | CN | CH₂OCH₂CH₃ | 3-Cl, 5-CF₃ |
| M.188. | CN | CH₂OCH₂CH₃ | 4-Cl, 3-CF₃ |
| M.189. | CN | CH₂SO₂CH₃ | — |
| M.190. | CN | CH₂SO₂CH₃ | 2-F |
| M.191. | CN | CH₂SO₂CH₃ | 3-F |
| M.192. | CN | CH₂SO₂CH₃ | 4-F |
| M.193. | CN | CH₂SO₂CH₃ | 2-Cl |
| M.194. | CN | CH₂SO₂CH₃ | 3-Cl |
| M.195. | CN | CH₂SO₂CH₃ | 4-Cl |
| M.196. | CN | CH₂SO₂CH₃ | 2-Br |
| M.197. | CN | CH₂SO₂CH₃ | 3-Br |
| M.198. | CN | CH₂SO₂CH₃ | 4-Br |
| M.199. | CN | CH₂SO₂CH₃ | 4-I |
| M.200. | CN | CH₂SO₂CH₃ | 2-CH₃ |
| M.201. | CN | CH₂SO₂CH₃ | 3-CH₃ |
| M.202. | CN | CH₂SO₂CH₃ | 4-CH₃ |
| M.203. | CN | CH₂SO₂CH₃ | 2-CF₃ |
| M.204. | CN | CH₂SO₂CH₃ | 3-CF₃ |
| M.205. | CN | CH₂SO₂CH₃ | 4-CF₃ |
| M.206. | CN | CH₂SO₂CH₃ | 2-OCF₃ |
| M.207. | CN | CH₂SO₂CH₃ | 3-OCF₃ |
| M.208. | CN | CH₂SO₂CH₃ | 4-OCF₃ |
| M.209. | CN | CH₂SO₂CH₃ | 4-OCHF₂ |
| M.210. | CN | CH₂SO₂CH₃ | 2-OCF₂O-3 |
| M.211. | CN | CH₂SO₂CH₃ | 3-OCF₂O-4 |
| M.212. | CN | CH₂SO₂CH₃ | 2-Cl, 4-O—CF₂—O-5 |
| M.213. | CN | CH₂SO₂CH₃ | 2-SCF₃ |
| M.214. | CN | CH₂SO₂CH₃ | 3-SCF₃ |
| M.215. | CN | CH₂SO₂CH₃ | 4-SCF₃ |
| M.216. | CN | CH₂SO₂CH₃ | 2-CN |
| M.217. | CN | CH₂SO₂CH₃ | 3-CN |
| M.218. | CN | CH₂SO₂CH₃ | 4-CN |
| M.219. | CN | CH₂SO₂CH₃ | 3-CN, 4-F |
| M.220. | CN | CH₂SO₂CH₃ | 3-CN, 4-Cl |
| M.221. | CN | CH₂SO₂CH₃ | 2-F, 5-CN |
| M.222. | CN | CH₂SO₂CH₃ | 2-Cl, 5-CN |
| M.223. | CN | CH₂SO₂CH₃ | 2-F, 4-CN |
| M.224. | CN | CH₂SO₂CH₃ | 2-Cl, 4-CN |
| M.225. | CN | CH₂SO₂CH₃ | 3-F, 4-CN |
| M.226. | CN | CH₂SO₂CH₃ | 3-Cl, 4-CN |
| M.227. | CN | CH₂SO₂CH₃ | 3-SO₂CH₃ |
| M.228. | CN | CH₂SO₂CH₃ | 4-SO₂CH₃ |
| M.229. | CN | CH₂SO₂CH₃ | 2-F, 4-SO₂CH₃ |
| M.230. | CN | CH₂SO₂CH₃ | 2-Cl, 4-SO₂CH₃ |
| M.231. | CN | CH₂SO₂CH₃ | 3-F, 4-SO₂CH₃ |
| M.232. | CN | CH₂SO₂CH₃ | 3-Cl, 4-SO₂CH₃ |
| M.233. | CN | CH₂SO₂CH₃ | 3-SO₂CH₃, 4-F |
| M.234. | CN | CH₂SO₂CH₃ | 3-SO₂CH₃, 4-Cl |
| M.235. | CN | CH₂SO₂CH₃ | 2-F, 5-SO₂CH₃ |
| M.236. | CN | CH₂SO₂CH₃ | 2-Cl, 5-SO₂CH₃ |
| M.237. | CN | CH₂SO₂CH₃ | 2,3-F₂ |
| M.238. | CN | CH₂SO₂CH₃ | 2,4-F₂ |
| M.239. | CN | CH₂SO₂CH₃ | 2,5-F₂ |
| M.240. | CN | CH₂SO₂CH₃ | 2,6-F₂ |
| M.241. | CN | CH₂SO₂CH₃ | 3,4-F₂ |
| M.242. | CN | CH₂SO₂CH₃ | 3,5-F₂ |
| M.243. | CN | CH₂SO₂CH₃ | 2,4-Cl₂ |
| M.244. | CN | CH₂SO₂CH₃ | 2,5-Cl₂ |
| M.245. | CN | CH₂SO₂CH₃ | 3,4-Cl₂ |
| M.246. | CN | CH₂SO₂CH₃ | 2,3,4-F₃ |
| M.247. | CN | CH₂SO₂CH₃ | 2,3,5-F₃ |
| M.248. | CN | CH₂SO₂CH₃ | 2,3,6-F₃ |
| M.249. | CN | CH₂SO₂CH₃ | 3,4,5-F₃ |
| M.250. | CN | CH₂SO₂CH₃ | 2,4,6-F₃ |
| M.251. | CN | CH₂SO₂CH₃ | 2-F, 3-Cl |
| M.252. | CN | CH₂SO₂CH₃ | 2-F, 4-Cl |
| M.253. | CN | CH₂SO₂CH₃ | 2-F, 5-Cl |
| M.254. | CN | CH₂SO₂CH₃ | 3-F, 4-Cl |
| M.255. | CN | CH₂SO₂CH₃ | 2-Cl, 4-F |
| M.256. | CN | CH₂SO₂CH₃ | 3-Cl, 4-F |
| M.257. | CN | CH₂SO₂CH₃ | 2-Cl, 4,5-F₂ |
| M.258. | CN | CH₂SO₂CH₃ | 2-Cl, 4-Br |
| M.259. | CN | CH₂SO₂CH₃ | 2-Cl, 5-Br |
| M.260. | CN | CH₂SO₂CH₃ | 3-Cl, 4-Br |
| M.261. | CN | CH₂SO₂CH₃ | 2-F, 4-Br |
| M.262. | CN | CH₂SO₂CH₃ | 2-F, 5-Br |
| M.263. | CN | CH₂SO₂CH₃ | 3-F, 4-Br |
| M.264. | CN | CH₂SO₂CH₃ | 2-Br, 4-F |
| M.265. | CN | CH₂SO₂CH₃ | 3-Br, 4-F |
| M.266. | CN | CH₂SO₂CH₃ | 2-Br, 5-Cl |
| M.267. | CN | CH₂SO₂CH₃ | 3-Br, 4-Cl |
| M.268. | CN | CH₂SO₂CH₃ | 2-Br, 4,5-F₂ |
| M.269. | CN | CH₂SO₂CH₃ | 3- ═N—O—N═ -4 |
| M.270. | CN | CH₂SO₂CH₃ | 4-CHF₂ |
| M.271. | CN | CH₂SO₂CH₃ | 2-F, 3-CF₃ |
| M.272. | CN | CH₂SO₂CH₃ | 2-F, 4-CF₃ |
| M.273. | CN | CH₂SO₂CH₃ | 2-F, 5-CF₃ |
| M.274. | CN | CH₂SO₂CH₃ | 3-F, 4-CF₃ |
| M.275. | CN | CH₂SO₂CH₃ | 3-F, 5-CF₃ |
| M.276. | CN | CH₂SO₂CH₃ | 4-F, 3-CF₃ |
| M.277. | CN | CH₂SO₂CH₃ | 3,4-F₂, 5-CF₃ |
| M.278. | CN | CH₂SO₂CH₃ | 2-Cl, 3-CF₃ |
| M.279. | CN | CH₂SO₂CH₃ | 2-Cl, 4-CF₃ |
| M.280. | CN | CH₂SO₂CH₃ | 2-Cl, 5-CF₃ |
| M.281. | CN | CH₂SO₂CH₃ | 3-Cl, 5-CF₃ |
| M.282. | CN | CH₂SO₂CH₃ | 4-Cl, 3-CF₃ |
| M.283. | CN | CH₂CN | — |
| M.284. | CN | CH₂CN | 2-F |
| M.285. | CN | CH₂CN | 3-F |
| M.286. | CN | CH₂CN | 4-F |
| M.287. | CN | CH₂CN | 2-Cl |
| M.288. | CN | CH₂CN | 3-Cl |
| M.289. | CN | CH₂CN | 4-Cl |
| M.290. | CN | CH₂CN | 2-Br |
| M.291. | CN | CH₂CN | 3-Br |
| M.292. | CN | CH₂CN | 4-Br |
| M.293. | CN | CH₂CN | 4-I |
| M.294. | CN | CH₂CN | 2-CH₃ |
| M.295. | CN | CH₂CN | 3-CH₃ |
| M.296. | CN | CH₂CN | 4-CH₃ |
| M.297. | CN | CH₂CN | 2-CF₃ |
| M.298. | CN | CH₂CN | 3-CF₃ |
| M.299. | CN | CH₂CN | 4-CF₃ |
| M.300. | CN | CH₂CN | 2-OCF₃ |
| M.301. | CN | CH₂CN | 3-OCF₃ |
| M.302. | CN | CH₂CN | 4-OCF₃ |
| M.303. | CN | CH₂CN | 4-OCHF₂ |
| M.304. | CN | CH₂CN | 2-OCF₂O-3 |
| M.305. | CN | CH₂CN | 3-OCF₂O-4 |
| M.306. | CN | CH₂CN | 2-Cl, 4-O—CF₂—O-5 |
| M.307. | CN | CH₂CN | 2-SCF₃ |
| M.308. | CN | CH₂CN | 3-SCF₃ |
| M.309. | CN | CH₂CN | 4-SCF₃ |
| M.310. | CN | CH₂CN | 2-CN |
| M.311. | CN | CH₂CN | 3-CN |
| M.312. | CN | CH₂CN | 4-CN |
| M.313. | CN | CH₂CN | 3-CN, 4-F |
| M.314. | CN | CH₂CN | 3-CN, 4-Cl |
| M.315. | CN | CH₂CN | 2-F, 5-CN |
| M.316. | CN | CH₂CN | 2-Cl, 5-CN |
| M.317. | CN | CH₂CN | 2-F, 4-CN |
| M.318. | CN | CH₂CN | 2-Cl, 4-CN |
| M.319. | CN | CH₂CN | 3-F, 4-CN |
| M.320. | CN | CH₂CN | 3-Cl, 4-CN |
| M.321. | CN | CH₂CN | 3-SO₂CH₃ |
| M.322. | CN | CH₂CN | 4-SO₂CH₃ |
| M.323. | CN | CH₂CN | 2-F, 4-SO₂CH₃ |
| M.324. | CN | CH₂CN | 2-Cl, 4-SO₂CH₃ |
| M.325. | CN | CH₂CN | 3-F, 4-SO₂CH₃ |
| M.326. | CN | CH₂CN | 3-Cl, 4-SO₂CH₃ |
| M.327. | CN | CH₂CN | 3-SO₂CH₃, 4-F |
| M.328. | CN | CH₂CN | 3-SO₂CH₃, 4-Cl |
| M.329. | CN | CH₂CN | 2-F, 5-SO₂CH₃ |
| M.330. | CN | CH₂CN | 2-Cl, 5-SO₂CH₃ |
| M.331. | CN | CH₂CN | 2,3-F₂ |
| M.332. | CN | CH₂CN | 2,4-F₂ |
| M.333. | CN | CH₂CN | 2,5-F₂ |
| M.334. | CN | CH₂CN | 2,6-F₂ |
| M.335. | CN | CH₂CN | 3,4-F₂ |
| M.336. | CN | CH₂CN | 3,5-F₂ |
| M.337. | CN | CH₂CN | 2,4-Cl₂ |
| M.338. | CN | CH₂CN | 2,5-Cl₂ |
| M.339. | CN | CH₂CN | 3,4-Cl₂ |
| M.340. | CN | CH₂CN | 2,3,4-F₃ |
| M.341. | CN | CH₂CN | 2,3,5-F₃ |
| M.342. | CN | CH₂CN | 2,3,6-F₃ |
| M.343. | CN | CH₂CN | 3,4,5-F₃ |
| M.344. | CN | CH₂CN | 2,4,6-F₃ |
| M.345. | CN | CH₂CN | 2-F, 3-Cl |
| M.346. | CN | CH₂CN | 2-F, 4-Cl |
| M.347. | CN | CH₂CN | 2-F, 5-Cl |
| M.348. | CN | CH₂CN | 3-F, 4-Cl |
| M.349. | CN | CH₂CN | 2-Cl, 4-F |
| M.350. | CN | CH₂CN | 3-Cl, 4-F |
| M.351. | CN | CH₂CN | 2-Cl, 4,5-F₂ |
| M.352. | CN | CH₂CN | 2-Cl, 4-Br |
| M.353. | CN | CH₂CN | 2-Cl, 5-Br |
| M.354. | CN | CH₂CN | 3-Cl, 4-Br |
| M.355. | CN | CH₂CN | 2-F, 4-Br |
| M.356. | CN | CH₂CN | 2-F, 5-Br |
| M.357. | CN | CH₂CN | 3-F, 4-Br |
| M.358. | CN | CH₂CN | 2-Br, 4-F |
| M.359. | CN | CH₂CN | 3-Br, 4-F |
| M.360. | CN | CH₂CN | 2-Br, 5-Cl |
| M.361. | CN | CH₂CN | 3-Br, 4-Cl |
| M.362. | CN | CH₂CN | 2-Br, 4,5-F₂ |
| M.363. | CN | CH₂CN | 3- ═N—O—N═ -4 |
| M.364. | CN | CH₂CN | 4-CHF₂ |
| M.365. | CN | CH₂CN | 2-F, 3-CF₃ |
| M.366. | CN | CH₂CN | 2-F, 4-CF₃ |
| M.367. | CN | CH₂CN | 2-F, 5-CF₃ |
| M.368. | CN | CH₂CN | 3-F, 4-CF₃ |
| M.369. | CN | CH₂CN | 3-F, 5-CF₃ |
| M.370. | CN | CH₂CN | 4-F, 3-CF₃ |
| M.371. | CN | CH₂CN | 3,4-F₂, 5-CF₃ |
| M.372. | CN | CH₂CN | 2-Cl, 3-CF₃ |
| M.373. | CN | CH₂CN | 2-Cl, 4-CF₃ |
| M.374. | CN | CH₂CN | 2-Cl, 5-CF₃ |
| M.375. | CN | CH₂CN | 3-Cl, 5-CF₃ |
| M.376. | CN | CH₂CN | 4-Cl, 3-CF₃ |
| M.377. | CN | C(CH₃)₂CN | — |
| M.378. | CN | C(CH₃)₂CN | 2-F |
| M.379. | CN | C(CH₃)₂CN | 3-F |
| M.380. | CN | C(CH₃)₂CN | 4-F |
| M.381. | CN | C(CH₃)₂CN | 2-Cl |
| M.382. | CN | C(CH₃)₂CN | 3-Cl |
| M.383. | CN | C(CH₃)₂CN | 4-Cl |
| M.384. | CN | C(CH₃)₂CN | 2-Br |
| M.385. | CN | C(CH₃)₂CN | 3-Br |
| M.386. | CN | C(CH₃)₂CN | 4-Br |
| M.387. | CN | C(CH₃)₂CN | 4-I |
| M.388. | CN | C(CH₃)₂CN | 2-CH₃ |
| M.389. | CN | C(CH₃)₂CN | 3-CH₃ |
| M.390. | CN | C(CH₃)₂CN | 4-CH₃ |
| M.391. | CN | C(CH₃)₂CN | 2-CF₃ |
| M.392. | CN | C(CH₃)₂CN | 3-CF₃ |
| M.393. | CN | C(CH₃)₂CN | 4-CF₃ |
| M.394. | CN | C(CH₃)₂CN | 2-OCF₃ |
| M.395. | CN | C(CH₃)₂CN | 3-OCF₃ |
| M.396. | CN | C(CH₃)₂CN | 4-OCF₃ |
| M.397. | CN | C(CH₃)₂CN | 4-OCHF₂ |
| M.398. | CN | C(CH₃)₂CN | 2-OCF₂O-3 |
| M.399. | CN | C(CH₃)₂CN | 3-OCF₂O-4 |
| M.400. | CN | C(CH₃)₂CN | 2-Cl, 4-O—CF₂—O-5 |
| M.401. | CN | C(CH₃)₂CN | 2-SCF₃ |
| M.402. | CN | C(CH₃)₂CN | 3-SCF₃ |
| M.403. | CN | C(CH₃)₂CN | 4-SCF₃ |
| M.404. | CN | C(CH₃)₂CN | 2-CN |
| M.405. | CN | C(CH₃)₂CN | 3-CN |
| M.406. | CN | C(CH₃)₂CN | 4-CN |
| M.407. | CN | C(CH₃)₂CN | 3-CN, 4-F |
| M.408. | CN | C(CH₃)₂CN | 3-CN, 4-Cl |
| M.409. | CN | C(CH₃)₂CN | 2-F, 5-CN |
| M.410. | CN | C(CH₃)₂CN | 2-Cl, 5-CN |
| M.411. | CN | C(CH₃)₂CN | 2-F, 4-CN |
| M.412. | CN | C(CH₃)₂CN | 2-Cl, 4-CN |
| M.413. | CN | C(CH₃)₂CN | 3-F, 4-CN |
| M.414. | CN | C(CH₃)₂CN | 3-Cl, 4-CN |
| M.415. | CN | C(CH₃)₂CN | 3-SO₂CH₃ |
| M.416. | CN | C(CH₃)₂CN | 4-SO₂CH₃ |
| M.417. | CN | C(CH₃)₂CN | 2-F, 4-SO₂CH₃ |
| M.418. | CN | C(CH₃)₂CN | 2-Cl, 4-SO₂CH₃ |
| M.419. | CN | C(CH₃)₂CN | 3-F, 4-SO₂CH₃ |
| M.420. | CN | C(CH₃)₂CN | 3-Cl, 4-SO₂CH₃ |
| M.421. | CN | C(CH₃)₂CN | 3-SO₂CH₃, 4-F |
| M.422. | CN | C(CH₃)₂CN | 3-SO₂CH₃, 4-Cl |
| M.423. | CN | C(CH₃)₂CN | 2-F, 5-SO₂CH₃ |
| M.424. | CN | C(CH₃)₂CN | 2-Cl, 5-SO₂CH₃ |
| M.425. | CN | C(CH₃)₂CN | 2,3-F₂ |
| M.426. | CN | C(CH₃)₂CN | 2,4-F₂ |
| M.427. | CN | C(CH₃)₂CN | 2,5-F₂ |
| M.428. | CN | C(CH₃)₂CN | 2,6-F₂ |
| M.429. | CN | C(CH₃)₂CN | 3,4-F₂ |
| M.430. | CN | C(CH₃)₂CN | 3,5-F₂ |
| M.431. | CN | C(CH₃)₂CN | 2,4-Cl₂ |
| M.432. | CN | C(CH₃)₂CN | 2,5-Cl₂ |
| M.433. | CN | C(CH₃)₂CN | 3,4-Cl₂ |
| M.434. | CN | C(CH₃)₂CN | 2,3,4-F₃ |
| M.435. | CN | C(CH₃)₂CN | 2,3,5-F₃ |
| M.436. | CN | C(CH₃)₂CN | 2,3,6-F₃ |
| M.437. | CN | C(CH₃)₂CN | 3,4,5-F₃ |
| M.438. | CN | C(CH₃)₂CN | 2,4,6-F₃ |
| M.439. | CN | C(CH₃)₂CN | 2-F, 3-Cl |
| M.440. | CN | C(CH₃)₂CN | 2-F, 4-Cl |
| M.441. | CN | C(CH₃)₂CN | 2-F, 5-Cl |
| M.442. | CN | C(CH₃)₂CN | 3-F, 4-Cl |
| M.443. | CN | C(CH₃)₂CN | 2-Cl, 4-F |
| M.444. | CN | C(CH₃)₂CN | 3-Cl, 4-F |
| M.445. | CN | C(CH₃)₂CN | 2-Cl, 4,5-F₂ |
| M.446. | CN | C(CH₃)₂CN | 2-Cl, 4-Br |
| M.447. | CN | C(CH₃)₂CN | 2-Cl, 5-Br |
| M.448. | CN | C(CH₃)₂CN | 3-Cl, 4-Br |
| M.449. | CN | C(CH₃)₂CN | 2-F, 4-Br |
| M.450. | CN | C(CH₃)₂CN | 2-F, 5-Br |
| M.451. | CN | C(CH₃)₂CN | 3-F, 4-Br |
| M.452. | CN | C(CH₃)₂CN | 2-Br, 4-F |
| M.453. | CN | C(CH₃)₂CN | 3-Br, 4-F |
| M.454. | CN | C(CH₃)₂CN | 2-Br, 5-Cl |
| M.455. | CN | C(CH₃)₂CN | 3-Br, 4-Cl |
| M.456. | CN | C(CH₃)₂CN | 2-Br, 4,5-F₂ |
| M.457. | CN | C(CH₃)₂CN | 3- ═N—O—N═ -4 |
| M.458. | CN | C(CH₃)₂CN | 4-CHF₂ |
| M.459. | CN | C(CH₃)₂CN | 2-F, 3-CF₃ |
| M.460. | CN | C(CH₃)₂CN | 2-F, 4-CF₃ |
| M.461. | CN | C(CH₃)₂CN | 2-F, 5-CF₃ |
| M.462. | CN | C(CH₃)₂CN | 3-F, 4-CF₃ |
| M.463. | CN | C(CH₃)₂CN | 3-F, 5-CF₃ |
| M.464. | CN | C(CH₃)₂CN | 4-F, 3-CF₃ |
| M.465. | CN | C(CH₃)₂CN | 3,4-F₂, 5-CF₃ |
| M.466. | CN | C(CH₃)₂CN | 2-Cl, 3-CF₃ |
| M.467. | CN | C(CH₃)₂CN | 2-Cl, 4-CF₃ |
| M.468. | CN | C(CH₃)₂CN | 2-Cl, 5-CF₃ |
| M.469. | CN | C(CH₃)₂CN | 3-Cl, 5-CF₃ |
| M.470. | CN | C(CH₃)₂CN | 4-Cl, 3-CF₃ |
| M.471. | CN | 1-cyanocyclopropyl | — |
| M.472. | CN | 1-cyanocyclopropyl | 2-F |
| M.473. | CN | 1-cyanocyclopropyl | 3-F |
| M.474. | CN | 1-cyanocyclopropyl | 4-F |
| M.475. | CN | 1-cyanocyclopropyl | 2-Cl |
| M.476. | CN | 1-cyanocyclopropyl | 3-Cl |
| M.477. | CN | 1-cyanocyclopropyl | 4-Cl |
| M.478. | CN | 1-cyanocyclopropyl | 2-Br |
| M.479. | CN | 1-cyanocyclopropyl | 3-Br |
| M.480. | CN | 1-cyanocyclopropyl | 4-Br |
| M.481. | CN | 1-cyanocyclopropyl | 4-I |
| M.482. | CN | 1-cyanocyclopropyl | 2-CH₃ |
| M.483. | CN | 1-cyanocyclopropyl | 3-CH₃ |
| M.484. | CN | 1-cyanocyclopropyl | 4-CH₃ |
| M.485. | CN | 1-cyanocyclopropyl | 2-CF₃ |
| M.486. | CN | 1-cyanocyclopropyl | 3-CF₃ |
| M.487. | CN | 1-cyanocyclopropyl | 4-CF₃ |
| M.488. | CN | 1-cyanocyclopropyl | 2-OCF₃ |
| M.489. | CN | 1-cyanocyclopropyl | 3-OCF₃ |
| M.490. | CN | 1-cyanocyclopropyl | 4-OCF₃ |
| M.491. | CN | 1-cyanocyclopropyl | 4-OCHF₂ |
| M.492. | CN | 1-cyanocyclopropyl | 2-OCF₂O-3 |
| M.493. | CN | 1-cyanocyclopropyl | 3-OCF₂O-4 |
| M.494. | CN | 1-cyanocyclopropyl | 2-Cl, 4-O—CF₂—O-5 |
| M.495. | CN | 1-cyanocyclopropyl | 2-SCF₃ |
| M.496. | CN | 1-cyanocyclopropyl | 3-SCF₃ |
| M.497. | CN | 1-cyanocyclopropyl | 4-SCF₃ |
| M.498. | CN | 1-cyanocyclopropyl | 2-CN |
| M.499. | CN | 1-cyanocyclopropyl | 3-CN |
| M.500. | CN | 1-cyanocyclopropyl | 4-CN |
| M.501. | CN | 1-cyanocyclopropyl | 3-CN, 4-F |
| M.502. | CN | 1-cyanocyclopropyl | 3-CN, 4-Cl |
| M.503. | CN | 1-cyanocyclopropyl | 2-F, 5-CN |
| M.504. | CN | 1-cyanocyclopropyl | 2-Cl, 5-CN |
| M.505. | CN | 1-cyanocyclopropyl | 2-F, 4-CN |
| M.506. | CN | 1-cyanocyclopropyl | 2-Cl, 4-CN |
| M.507. | CN | 1-cyanocyclopropyl | 3-F, 4-CN |
| M.508. | CN | 1-cyanocyclopropyl | 3-Cl, 4-CN |
| M.509. | CN | 1-cyanocyclopropyl | 3-SO₂CH₃ |
| M.510. | CN | 1-cyanocyclopropyl | 4-SO₂CH₃ |
| M.511. | CN | 1-cyanocyclopropyl | 2-F, 4-SO₂CH₃ |
| M.512. | CN | 1-cyanocyclopropyl | 2-Cl, 4-SO₂CH₃ |
| M.513. | CN | 1-cyanocyclopropyl | 3-F, 4-SO₂CH₃ |
| M.514. | CN | 1-cyanocyclopropyl | 3-Cl, 4-SO₂CH₃ |
| M.515. | CN | 1-cyanocyclopropyl | 3-SO₂CH₃, 4-F |
| M.516. | CN | 1-cyanocyclopropyl | 3-SO₂CH₃, 4-Cl |
| M.517. | CN | 1-cyanocyclopropyl | 2-F, 5-SO₂CH₃ |
| M.518. | CN | 1-cyanocyclopropyl | 2-Cl, 5-SO₂CH₃ |
| M.519. | CN | 1-cyanocyclopropyl | 2,3-F₂ |
| M.520. | CN | 1-cyanocyclopropyl | 2,4-F₂ |
| M.521. | CN | 1-cyanocyclopropyl | 2,5-F₂ |
| M.522. | CN | 1-cyanocyclopropyl | 2,6-F₂ |
| M.523. | CN | 1-cyanocyclopropyl | 3,4-F₂ |
| M.524. | CN | 1-cyanocyclopropyl | 3,5-F₂ |
| M.525. | CN | 1-cyanocyclopropyl | 2,4-Cl₂ |
| M.526. | CN | 1-cyanocyclopropyl | 2,5-Cl₂ |
| M.527. | CN | 1-cyanocyclopropyl | 3,4-Cl₂ |
| M.528. | CN | 1-cyanocyclopropyl | 2,3,4-F₃ |
| M.529. | CN | 1-cyanocyclopropyl | 2,3,5-F₃ |
| M.530. | CN | 1-cyanocyclopropyl | 2,3,6-F₃ |
| M.531. | CN | 1-cyanocyclopropyl | 3,4,5-F₃ |
| M.532. | CN | 1-cyanocyclopropyl | 2,4,6-F₃ |
| M.533. | CN | 1-cyanocyclopropyl | 2-F, 3-Cl |
| M.534. | CN | 1-cyanocyclopropyl | 2-F, 4-Cl |
| M.535. | CN | 1-cyanocyclopropyl | 2-F, 5-Cl |
| M.536. | CN | 1-cyanocyclopropyl | 3-F, 4-Cl |
| M.537. | CN | 1-cyanocyclopropyl | 2-Cl, 4-F |
| M.538. | CN | 1-cyanocyclopropyl | 3-Cl, 4-F |
| M.539. | CN | 1-cyanocyclopropyl | 2-Cl, 4,5-F₂ |
| M.540. | CN | 1-cyanocyclopropyl | 2-Cl, 4-Br |
| M.541. | CN | 1-cyanocyclopropyl | 2-Cl, 5-Br |
| M.542. | CN | 1-cyanocyclopropyl | 3-Cl, 4-Br |
| M.543. | CN | 1-cyanocyclopropyl | 2-F, 4-Br |
| M.544. | CN | 1-cyanocyclopropyl | 2-F, 5-Br |
| M.545. | CN | 1-cyanocyclopropyl | 3-F, 4-Br |
| M.546. | CN | 1-cyanocyclopropyl | 2-Br, 4-F |
| M.547. | CN | 1-cyanocyclopropyl | 3-Br, 4-F |
| M.548. | CN | 1-cyanocyclopropyl | 2-Br, 5-Cl |
| M.549. | CN | 1-cyanocyclopropyl | 3-Br, 4-Cl |
| M.550. | CN | 1-cyanocyclopropyl | 2-Br, 4,5-F₂ |
| M.551. | CN | 1-cyanocyclopropyl | 3- ═N—O—N═ -4 |
| M.552. | CN | 1-cyanocyclopropyl | 4-CHF₂ |
| M.553. | CN | 1-cyanocyclopropyl | 2-F, 3-CF₃ |
| M.554. | CN | 1-cyanocyclopropyl | 2-F, 4-CF₃ |
| M.555. | CN | 1-cyanocyclopropyl | 2-F, 5-CF₃ |
| M.556. | CN | 1-cyanocyclopropyl | 3-F, 4-CF₃ |
| M.557. | CN | 1-cyanocyclopropyl | 3-F, 5-CF₃ |
| M.558. | CN | 1-cyanocyclopropyl | 4-F, 3-CF₃ |
| M.559. | CN | 1-cyanocyclopropyl | 3,4-F₂, 5-CF₃ |
| M.560. | CN | 1-cyanocyclopropyl | 2-Cl, 3-CF₃ |
| M.561. | CN | 1-cyanocyclopropyl | 2-Cl, 4-CF₃ |
| M.562. | CN | 1-cyanocyclopropyl | 2-Cl, 5-CF₃ |
| M.563. | CN | 1-cyanocyclopropyl | 3-Cl, 5-CF₃ |
| M.564. | CN | 1-cyanocyclopropyl | 4-Cl, 3-CF₃ |
| M.565. | CN | 1-methylsulfonylcyclopropyl | — |
| M.566. | CN | 1-methylsulfonylcyclopropyl | 2-F |
| M.567. | CN | 1-methylsulfonylcyclopropyl | 3-F |
| M.568. | CN | 1-methylsulfonylcyclopropyl | 4-F |
| M.569. | CN | 1-methylsulfonylcyclopropyl | 2-Cl |
| M.570. | CN | 1-methylsulfonylcyclopropyl | 3-Cl |
| M.571. | CN | 1-methylsulfonylcyclopropyl | 4-Cl |
| M.572. | CN | 1-methylsulfonylcyclopropyl | 2-Br |
| M.573. | CN | 1-methylsulfonylcyclopropyl | 3-Br |
| M.574. | CN | 1-methylsulfonylcyclopropyl | 4-Br |
| M.575. | CN | 1-methylsulfonylcyclopropyl | 4-I |
| M.576. | CN | 1-methylsulfonylcyclopropyl | 2-CH₃ |
| M.577. | CN | 1-methylsulfonylcyclopropyl | 3-CH₃ |
| M.578. | CN | 1-methylsulfonylcyclopropyl | 4-CH₃ |
| M.579. | CN | 1-methylsulfonylcyclopropyl | 2-CF₃ |
| M.580. | CN | 1-methylsulfonylcyclopropyl | 3-CF₃ |
| M.581. | CN | 1-methylsulfonylcyclopropyl | 4-CF₃ |
| M.582. | CN | 1-methylsulfonylcyclopropyl | 2-OCF₃ |
| M.583. | CN | 1-methylsulfonylcyclopropyl | 3-OCF₃ |
| M.584. | CN | 1-methylsulfonylcyclopropyl | 4-OCF₃ |
| M.585. | CN | 1-methylsulfonylcyclopropyl | 4-OCHF₂ |
| M.586. | CN | 1-methylsulfonylcyclopropyl | 2-OCF₂O-3 |
| M.587. | CN | 1-methylsulfonylcyclopropyl | 3-OCF₂O-4 |
| M.588. | CN | 1-methylsulfonylcyclopropyl | 2-Cl, 4-O—CF₂—O-5 |
| M.589. | CN | 1-methylsulfonylcyclopropyl | 2-SCF₃ |
| M.590. | CN | 1-methylsulfonylcyclopropyl | 3-SCF₃ |
| M.591. | CN | 1-methylsulfonylcyclopropyl | 4-SCF₃ |
| M.592. | CN | 1-methylsulfonylcyclopropyl | 2-CN |
| M.593. | CN | 1-methylsulfonylcyclopropyl | 3-CN |
| M.594. | CN | 1-methylsulfonylcyclopropyl | 4-CN |
| M.595. | CN | 1-methylsulfonylcyclopropyl | 3-CN, 4-F |
| M.596. | CN | 1-methylsulfonylcyclopropyl | 3-CN, 4-Cl |
| M.597. | CN | 1-methylsulfonylcyclopropyl | 2-F, 5-CN |
| M.598. | CN | 1-methylsulfonylcyclopropyl | 2-Cl, 5-CN |
| M.599. | CN | 1-methylsulfonylcyclopropyl | 2-F, 4-CN |
| M.600. | CN | 1-methylsulfonylcyclopropyl | 2-Cl, 4-CN |
| M.601. | CN | 1-methylsulfonylcyclopropyl | 3-F, 4-CN |
| M.602. | CN | 1-methylsulfonylcyclopropyl | 3-Cl, 4-CN |
| M.603. | CN | 1-methylsulfonylcyclopropyl | 3-SO₂CH₃ |
| M.604. | CN | 1-methylsulfonylcyclopropyl | 4-SO₂CH₃ |
| M.605. | CN | 1-methylsulfonylcyclopropyl | 2-F, 4-SO₂CH₃ |
| M.606. | CN | 1-methylsulfonylcyclopropyl | 2-Cl, 4-SO₂CH₃ |
| M.607. | CN | 1-methylsulfonylcyclopropyl | 3-F, 4-SO₂CH₃ |
| M.608. | CN | 1-methylsulfonylcyclopropyl | 3-Cl, 4-SO₂CH₃ |
| M.609. | CN | 1-methylsulfonylcyclopropyl | 3-SO₂CH₃, 4-F |
| M.610. | CN | 1-methylsulfonylcyclopropyl | 3-SO₂CH₃, 4-Cl |
| M.611. | CN | 1-methylsulfonylcyclopropyl | 2-F, 5-SO₂CH₃ |
| M.612. | CN | 1-methylsulfonylcyclopropyl | 2-Cl, 5-SO₂CH₃ |
| M.613. | CN | 1-methylsulfonylcyclopropyl | 2,3-F₂ |
| M.614. | CN | 1-methylsulfonylcyclopropyl | 2,4-F₂ |
| M.615. | CN | 1-methylsulfonylcyclopropyl | 2,5-F₂ |
| M.616. | CN | 1-methylsulfonylcyclopropyl | 2,6-F₂ |
| M.617. | CN | 1-methylsulfonylcyclopropyl | 3,4-F₂ |
| M.618. | CN | 1-methylsulfonylcyclopropyl | 3,5-F₂ |
| M.619. | CN | 1-methylsulfonylcyclopropyl | 2,4-Cl₂ |
| M.620. | CN | 1-methylsulfonylcyclopropyl | 2,5-Cl₂ |
| M.621. | CN | 1-methylsulfonylcyclopropyl | 3,4-Cl₂ |
| M.622. | CN | 1-methylsulfonylcyclopropyl | 2,3,4-F₃ |
| M.623. | CN | 1-methylsulfonylcyclopropyl | 2,3,5-F₃ |
| M.624. | CN | 1-methylsulfonylcyclopropyl | 2,3,6-F₃ |
| M.625. | CN | 1-methylsulfonylcyclopropyl | 3,4,5-F₃ |
| M.626. | CN | 1-methylsulfonylcyclopropyl | 2,4,6-F₃ |
| M.627. | CN | 1-methylsulfonylcyclopropyl | 2-F, 3-Cl |
| M.628. | CN | 1-methylsulfonylcyclopropyl | 2-F, 4-Cl |
| M.629. | CN | 1-methylsulfonylcyclopropyl | 2-F, 5-Cl |
| M.630. | CN | 1-methylsulfonylcyclopropyl | 3-F, 4-Cl |
| M.631. | CN | 1-methylsulfonylcyclopropyl | 2-Cl, 4-F |
| M.632. | CN | 1-methylsulfonylcyclopropyl | 3-Cl, 4-F |
| M.633. | CN | 1-methylsulfonylcyclopropyl | 2-Cl, 4,5-F₂ |
| M.634. | CN | 1-methylsulfonylcyclopropyl | 2-Cl, 4-Br |
| M.635. | CN | 1-methylsulfonylcyclopropyl | 2-Cl, 5-Br |
| M.636. | CN | 1-methylsulfonylcyclopropyl | 3-Cl, 4-Br |
| M.637. | CN | 1-methylsulfonylcyclopropyl | 2-F, 4-Br |
| M.638. | CN | 1-methylsulfonylcyclopropyl | 2-F, 5-Br |
| M.639. | CN | 1-methylsulfonylcyclopropyl | 3-F, 4-Br |
| M.640. | CN | 1-methylsulfonylcyclopropyl | 2-Br, 4-F |
| M.641. | CN | 1-methylsulfonylcyclopropyl | 3-Br, 4-F |
| M.642. | CN | 1-methylsulfonylcyclopropyl | 2-Br, 5-Cl |
| M.643. | CN | 1-methylsulfonylcyclopropyl | 3-Br, 4-Cl |
| M.644. | CN | 1-methylsulfonylcyclopropyl | 2-Br, 4,5-F₂ |
| M.645. | CN | 1-methylsulfonylcyclopropyl | 3- ═N—O—N═ -4 |
| M.646. | CN | 1-methylsulfonylcyclopropyl | 4-CHF₂ |
| M.647. | CN | 1-methylsulfonylcyclopropyl | 2-F, 3-CF₃ |
| M.648. | CN | 1-methylsulfonylcyclopropyl | 2-F, 4-CF₃ |
| M.649. | CN | 1-methylsulfonylcyclopropyl | 2-F, 5-CF₃ |
| M.650. | CN | 1-methylsulfonylcyclopropyl | 3-F, 4-CF₃ |
| M.651. | CN | 1-methylsulfonylcyclopropyl | 3-F, 5-CF₃ |
| M.652. | CN | 1-methylsulfonylcyclopropyl | 4-F, 3-CF₃ |
| M.653. | CN | 1-methylsulfonylcyclopropyl | 3,4-F₂, 5-CF₃ |
| M.654. | CN | 1-methylsulfonylcyclopropyl | 2-Cl, 3-CF₃ |
| M.655. | CN | 1-methylsulfonylcyclopropyl | 2-Cl, 4-CF₃ |
| M.656. | CN | 1-methylsulfonylcyclopropyl | 2-Cl, 5-CF₃ |
| M.657. | CN | 1-methylsulfonylcyclopropyl | 3-Cl, 5-CF₃ |
| M.658. | CN | 1-methylsulfonylcyclopropyl | 4-Cl, 3-CF₃ |
| M.659. | C(═S)NH₂ | CH₂OCH₃ | — |
| M.660. | C(═S)NH₂ | CH₂OCH₃ | 2-F |
| M.661. | C(═S)NH₂ | CH₂OCH₃ | 3-F |
| M.662. | C(═S)NH₂ | CH₂OCH₃ | 4-F |
| M.663. | C(═S)NH₂ | CH₂OCH₃ | 2-Cl |
| M.664. | C(═S)NH₂ | CH₂OCH₃ | 3-Cl |
| M.665. | C(═S)NH₂ | CH₂OCH₃ | 4-Cl |
| M.666. | C(═S)NH₂ | CH₂OCH₃ | 2-Br |
| M.667. | C(═S)NH₂ | CH₂OCH₃ | 3-Br |
| M.668. | C(═S)NH₂ | CH₂OCH₃ | 4-Br |
| M.669. | C(═S)NH₂ | CH₂OCH₃ | 4-I |
| M.670. | C(═S)NH₂ | CH₂OCH₃ | 2-CH₃ |
| M.671. | C(═S)NH₂ | CH₂OCH₃ | 3-CH₃ |
| M.672. | C(═S)NH₂ | CH₂OCH₃ | 4-CH₃ |
| M.673. | C(═S)NH₂ | CH₂OCH₃ | 2-CF₃ |
| M.674. | C(═S)NH₂ | CH₂OCH₃ | 3-CF₃ |
| M.675. | C(═S)NH₂ | CH₂OCH₃ | 4-CF₃ |
| M.676. | C(═S)NH₂ | CH₂OCH₃ | 2-OCF₃ |
| M.677. | C(═S)NH₂ | CH₂OCH₃ | 3-OCF₃ |
| M.678. | C(═S)NH₂ | CH₂OCH₃ | 4-OCF₃ |
| M.679. | C(═S)NH₂ | CH₂OCH₃ | 4-OCHF₂ |
| M.680. | C(═S)NH₂ | CH₂OCH₃ | 2-OCF₂O-3 |
| M.681. | C(═S)NH₂ | CH₂OCH₃ | 3-OCF₂O-4 |
| M.682. | C(═S)NH₂ | CH₂OCH₃ | 2-Cl, 4-O—CF₂—O-5 |
| M.683. | C(═S)NH₂ | CH₂OCH₃ | 2-SCF₃ |
| M.684. | C(═S)NH₂ | CH₂OCH₃ | 3-SCF₃ |
| M.685. | C(═S)NH₂ | CH₂OCH₃ | 4-SCF₃ |
| M.686. | C(═S)NH₂ | CH₂OCH₃ | 2-CN |
| M.687. | C(═S)NH₂ | CH₂OCH₃ | 3-CN |
| M.688. | C(═S)NH₂ | CH₂OCH₃ | 4-CN |
| M.689. | C(═S)NH₂ | CH₂OCH₃ | 3-CN, 4-F |
| M.690. | C(═S)NH₂ | CH₂OCH₃ | 3-CN, 4-Cl |
| M.691. | C(═S)NH₂ | CH₂OCH₃ | 2-F, 5-CN |
| M.692. | C(═S)NH₂ | CH₂OCH₃ | 2-Cl, 5-CN |
| M.693. | C(═S)NH₂ | CH₂OCH₃ | 2-F, 4-CN |
| M.694. | C(═S)NH₂ | CH₂OCH₃ | 2-Cl, 4-CN |
| M.695. | C(═S)NH₂ | CH₂OCH₃ | 3-F, 4-CN |
| M.696. | C(═S)NH₂ | CH₂OCH₃ | 3-Cl, 4-CN |
| M.697. | C(═S)NH₂ | CH₂OCH₃ | 3-SO₂CH₃ |
| M.698. | C(═S)NH₂ | CH₂OCH₃ | 4-SO₂CH₃ |
| M.699. | C(═S)NH₂ | CH₂OCH₃ | 2-F, 4-SO₂CH₃ |
| M.700. | C(═S)NH₂ | CH₂OCH₃ | 2-Cl, 4-SO₂CH₃ |
| M.701. | C(═S)NH₂ | CH₂OCH₃ | 3-F, 4-SO₂CH₃ |
| M.702. | C(═S)NH₂ | CH₂OCH₃ | 3-Cl, 4-SO₂CH₃ |
| M.703. | C(═S)NH₂ | CH₂OCH₃ | 3-SO₂CH₃, 4-F |
| M.704. | C(═S)NH₂ | CH₂OCH₃ | 3-SO₂CH₃, 4-Cl |
| M.705. | C(═S)NH₂ | CH₂OCH₃ | 2-F, 5-SO₂CH₃ |
| M.706. | C(═S)NH₂ | CH₂OCH₃ | 2-Cl, 5-SO₂CH₃ |
| M.707. | C(═S)NH₂ | CH₂OCH₃ | 2,3-F₂ |
| M.708. | C(═S)NH₂ | CH₂OCH₃ | 2,4-F₂ |
| M.709. | C(═S)NH₂ | CH₂OCH₃ | 2,5-F₂ |
| M.710. | C(═S)NH₂ | CH₂OCH₃ | 2,6-F₂ |
| M.711. | C(═S)NH₂ | CH₂OCH₃ | 3,4-F₂ |
| M.712. | C(═S)NH₂ | CH₂OCH₃ | 3,5-F₂ |
| M.713. | C(═S)NH₂ | CH₂OCH₃ | 2,4-Cl₂ |
| M.714. | C(═S)NH₂ | CH₂OCH₃ | 2,5-Cl₂ |
| M.715. | C(═S)NH₂ | CH₂OCH₃ | 3,4-Cl₂ |
| M.716. | C(═S)NH₂ | CH₂OCH₃ | 2,3,4-F₃ |
| M.717. | C(═S)NH₂ | CH₂OCH₃ | 2,3,5-F₃ |
| M.718. | C(═S)NH₂ | CH₂OCH₃ | 2,3,6-F₃ |
| M.719. | C(═S)NH₂ | CH₂OCH₃ | 3,4,5-F₃ |
| M.720. | C(═S)NH₂ | CH₂OCH₃ | 2,4,6-F₃ |
| M.721. | C(═S)NH₂ | CH₂OCH₃ | 2-F, 3-Cl |
| M.722. | C(═S)NH₂ | CH₂OCH₃ | 2-F, 4-Cl |
| M.723. | C(═S)NH₂ | CH₂OCH₃ | 2-F, 5-Cl |
| M.724. | C(═S)NH₂ | CH₂OCH₃ | 3-F, 4-Cl |
| M.725. | C(═S)NH₂ | CH₂OCH₃ | 2-Cl, 4-F |
| M.726. | C(═S)NH₂ | CH₂OCH₃ | 3-Cl, 4-F |
| M.727. | C(═S)NH₂ | CH₂OCH₃ | 2-Cl, 4,5-F₂ |
| M.728. | C(═S)NH₂ | CH₂OCH₃ | 2-Cl, 4-Br |
| M.729. | C(═S)NH₂ | CH₂OCH₃ | 2-Cl, 5-Br |
| M.730. | C(═S)NH₂ | CH₂OCH₃ | 3-Cl, 4-Br |
| M.731. | C(═S)NH₂ | CH₂OCH₃ | 2-F, 4-Br |
| M.732. | C(═S)NH₂ | CH₂OCH₃ | 2-F, 5-Br |
| M.733. | C(═S)NH₂ | CH₂OCH₃ | 3-F, 4-Br |
| M.734. | C(═S)NH₂ | CH₂OCH₃ | 2-Br, 4-F |
| M.735. | C(═S)NH₂ | CH₂OCH₃ | 3-Br, 4-F |
| M.736. | C(═S)NH₂ | CH₂OCH₃ | 2-Br, 5-Cl |
| M.737. | C(═S)NH₂ | CH₂OCH₃ | 3-Br, 4-Cl |
| M.738. | C(═S)NH₂ | CH₂OCH₃ | 2-Br, 4,5-F₂ |
| M.739. | C(═S)NH₂ | CH₂OCH₃ | 3- ═N—O—N═ -4 |
| M.740. | C(═S)NH₂ | CH₂OCH₃ | 4-CHF₂ |
| M.741. | C(═S)NH₂ | CH₂OCH₃ | 2-F, 3-CF₃ |
| M.742. | C(═S)NH₂ | CH₂OCH₃ | 2-F, 4-CF₃ |
| M.743. | C(═S)NH₂ | CH₂OCH₃ | 2-F, 5-CF₃ |
| M.744. | C(═S)NH₂ | CH₂OCH₃ | 3-F, 4-CF₃ |
| M.745. | C(═S)NH₂ | CH₂OCH₃ | 3-F, 5-CF₃ |
| M.746. | C(═S)NH₂ | CH₂OCH₃ | 4-F, 3-CF₃ |
| M.747. | C(═S)NH₂ | CH₂OCH₃ | 3,4-F₂, 5-CF₃ |
| M.748. | C(═S)NH₂ | CH₂OCH₃ | 2-Cl, 3-CF₃ |
| M.749. | C(═S)NH₂ | CH₂OCH₃ | 2-Cl, 4-CF₃ |
| M.750. | C(═S)NH₂ | CH₂OCH₃ | 2-Cl, 5-CF₃ |
| M.751. | C(═S)NH₂ | CH₂OCH₃ | 3-Cl, 5-CF₃ |
| M.752. | C(═S)NH₂ | CH₂OCH₃ | 4-Cl, 3-CF₃ |
| M.753. | C(═S)NH₂ | CH₂OCH₂CH₃ | — |
| M.754. | C(═S)NH₂ | CH₂OCH₂CH₃ | 2-F |
| M.755. | C(═S)NH₂ | CH₂OCH₂CH₃ | 3-F |
| M.756. | C(═S)NH₂ | CH₂OCH₂CH₃ | 4-F |
| M.757. | C(═S)NH₂ | CH₂OCH₂CH₃ | 2-Cl |
| M.758. | C(═S)NH₂ | CH₂OCH₂CH₃ | 3-Cl |
| M.759. | C(═S)NH₂ | CH₂OCH₂CH₃ | 4-Cl |
| M.760. | C(═S)NH₂ | CH₂OCH₂CH₃ | 2-Br |
| M.761. | C(═S)NH₂ | CH₂OCH₂CH₃ | 3-Br |
| M.762. | C(═S)NH₂ | CH₂OCH₂CH₃ | 4-Br |
| M.763. | C(═S)NH₂ | CH₂OCH₂CH₃ | 4-I |
| M.764. | C(═S)NH₂ | CH₂OCH₂CH₃ | 2-CH₃ |
| M.765. | C(═S)NH₂ | CH₂OCH₂CH₃ | 3-CH₃ |
| M.766. | C(═S)NH₂ | CH₂OCH₂CH₃ | 4-CH₃ |
| M.767. | C(═S)NH₂ | CH₂OCH₂CH₃ | 2-CF₃ |
| M.768. | C(═S)NH₂ | CH₂OCH₂CH₃ | 3-CF₃ |
| M.769. | C(═S)NH₂ | CH₂OCH₂CH₃ | 4-CF₃ |
| M.770. | C(═S)NH₂ | CH₂OCH₂CH₃ | 2-OCF₃ |
| M.771. | C(═S)NH₂ | CH₂OCH₂CH₃ | 3-OCF₃ |
| M.772. | C(═S)NH₂ | CH₂OCH₂CH₃ | 4-OCF₃ |
| M.773. | C(═S)NH₂ | CH₂OCH₂CH₃ | 4-OCHF₂ |
| M.774. | C(═S)NH₂ | CH₂OCH₂CH₃ | 2-OCF₂O-3 |
| M.775. | C(═S)NH₂ | CH₂OCH₂CH₃ | 3-OCF₂O-4 |
| M.776. | C(═S)NH₂ | CH₂OCH₂CH₃ | 2-Cl, 4-O—CF₂—O-5 |
| M.777. | C(═S)NH₂ | CH₂OCH₂CH₃ | 2-SCF₃ |
| M.778. | C(═S)NH₂ | CH₂OCH₂CH₃ | 3-SCF₃ |
| M.779. | C(═S)NH₂ | CH₂OCH₂CH₃ | 4-SCF₃ |
| M.780. | C(═S)NH₂ | CH₂OCH₂CH₃ | 2-CN |
| M.781. | C(═S)NH₂ | CH₂OCH₂CH₃ | 3-CN |
| M.782. | C(═S)NH₂ | CH₂OCH₂CH₃ | 4-CN |
| M.783. | C(═S)NH₂ | CH₂OCH₂CH₃ | 3-CN, 4-F |
| M.784. | C(═S)NH₂ | CH₂OCH₂CH₃ | 3-CN, 4-Cl |
| M.785. | C(═S)NH₂ | CH₂OCH₂CH₃ | 2-F, 5-CN |
| M.786. | C(═S)NH₂ | CH₂OCH₂CH₃ | 2-Cl, 5-CN |
| M.787. | C(═S)NH₂ | CH₂OCH₂CH₃ | 2-F, 4-CN |
| M.788. | C(═S)NH₂ | CH₂OCH₂CH₃ | 2-Cl, 4-CN |
| M.789. | C(═S)NH₂ | CH₂OCH₂CH₃ | 3-F, 4-CN |
| M.790. | C(═S)NH₂ | CH₂OCH₂CH₃ | 3-Cl, 4-CN |
| M.791. | C(═S)NH₂ | CH₂OCH₂CH₃ | 3-SO₂CH₃ |
| M.792. | C(═S)NH₂ | CH₂OCH₂CH₃ | 4-SO₂CH₃ |
| M.793. | C(═S)NH₂ | CH₂OCH₂CH₃ | 2-F, 4-SO₂CH₃ |
| M.794. | C(═S)NH₂ | CH₂OCH₂CH₃ | 2-Cl, 4-SO₂CH₃ |
| M.795. | C(═S)NH₂ | CH₂OCH₂CH₃ | 3-F, 4-SO₂CH₃ |
| M.796. | C(═S)NH₂ | CH₂OCH₂CH₃ | 3-Cl, 4-SO₂CH₃ |
| M.797. | C(═S)NH₂ | CH₂OCH₂CH₃ | 3-SO₂CH₃, 4-F |
| M.798. | C(═S)NH₂ | CH₂OCH₂CH₃ | 3-SO₂CH₃, 4-Cl |
| M.799. | C(═S)NH₂ | CH₂OCH₂CH₃ | 2-F, 5-SO₂CH₃ |
| M.800. | C(═S)NH₂ | CH₂OCH₂CH₃ | 2-Cl, 5-SO₂CH₃ |
| M.801. | C(═S)NH₂ | CH₂OCH₂CH₃ | 2,3-F₂ |
| M.802. | C(═S)NH₂ | CH₂OCH₂CH₃ | 2,4-F₂ |
| M.803. | C(═S)NH₂ | CH₂OCH₂CH₃ | 2,5-F₂ |
| M.804. | C(═S)NH₂ | CH₂OCH₂CH₃ | 2,6-F₂ |
| M.805. | C(═S)NH₂ | CH₂OCH₂CH₃ | 3,4-F₂ |
| M.806. | C(═S)NH₂ | CH₂OCH₂CH₃ | 3,5-F₂ |
| M.807. | C(═S)NH₂ | CH₂OCH₂CH₃ | 2,4-Cl₂ |
| M.808. | C(═S)NH₂ | CH₂OCH₂CH₃ | 2,5-Cl₂ |
| M.809. | C(═S)NH₂ | CH₂OCH₂CH₃ | 3,4-Cl₂ |
| M.810. | C(═S)NH₂ | CH₂OCH₂CH₃ | 2,3,4-F₃ |
| M.811. | C(═S)NH₂ | CH₂OCH₂CH₃ | 2,3,5-F₃ |
| M.812. | C(═S)NH₂ | CH₂OCH₂CH₃ | 2,3,6-F₃ |
| M.813. | C(═S)NH₂ | CH₂OCH₂CH₃ | 3,4,5-F₃ |
| M.814. | C(═S)NH₂ | CH₂OCH₂CH₃ | 2,4,6-F₃ |
| M.815. | C(═S)NH₂ | CH₂OCH₂CH₃ | 2-F, 3-Cl |
| M.816. | C(═S)NH₂ | CH₂OCH₂CH₃ | 2-F, 4-Cl |
| M.817. | C(═S)NH₂ | CH₂OCH₂CH₃ | 2-F, 5-Cl |
| M.818. | C(═S)NH₂ | CH₂OCH₂CH₃ | 3-F, 4-Cl |
| M.819. | C(═S)NH₂ | CH₂OCH₂CH₃ | 2-Cl, 4-F |
| M.820. | C(═S)NH₂ | CH₂OCH₂CH₃ | 3-Cl, 4-F |
| M.821. | C(═S)NH₂ | CH₂OCH₂CH₃ | 2-Cl, 4,5-F₂ |
| M.822. | C(═S)NH₂ | CH₂OCH₂CH₃ | 2-Cl, 4-Br |
| M.823. | C(═S)NH₂ | CH₂OCH₂CH₃ | 2-Cl, 5-Br |
| M.824. | C(═S)NH₂ | CH₂OCH₂CH₃ | 3-Cl, 4-Br |
| M.825. | C(═S)NH₂ | CH₂OCH₂CH₃ | 2-F, 4-Br |
| M.826. | C(═S)NH₂ | CH₂OCH₂CH₃ | 2-F, 5-Br |
| M.827. | C(═S)NH₂ | CH₂OCH₂CH₃ | 3-F, 4-Br |
| M.828. | C(═S)NH₂ | CH₂OCH₂CH₃ | 2-Br, 4-F |
| M.829. | C(═S)NH₂ | CH₂OCH₂CH₃ | 3-Br, 4-F |
| M.830. | C(═S)NH₂ | CH₂OCH₂CH₃ | 2-Br, 5-Cl |
| M.831. | C(═S)NH₂ | CH₂OCH₂CH₃ | 3-Br, 4-Cl |
| M.832. | C(═S)NH₂ | CH₂OCH₂CH₃ | 2-Br, 4,5-F₂ |
| M.833. | C(═S)NH₂ | CH₂OCH₂CH₃ | 3- ═N—O—N═ -4 |
| M.834. | C(═S)NH₂ | CH₂OCH₂CH₃ | 4-CHF₂ |
| M.835. | C(═S)NH₂ | CH₂OCH₂CH₃ | 2-F, 3-CF₃ |
| M.836. | C(═S)NH₂ | CH₂OCH₂CH₃ | 2-F, 4-CF₃ |
| M.837. | C(═S)NH₂ | CH₂OCH₂CH₃ | 2-F, 5-CF₃ |
| M.838. | C(═S)NH₂ | CH₂OCH₂CH₃ | 3-F, 4-CF₃ |
| M.839. | C(═S)NH₂ | CH₂OCH₂CH₃ | 3-F, 5-CF₃ |
| M.840. | C(═S)NH₂ | CH₂OCH₂CH₃ | 4-F, 3-CF₃ |
| M.841. | C(═S)NH₂ | CH₂OCH₂CH₃ | 3,4-F₂, 5-CF₃ |
| M.842. | C(═S)NH₂ | CH₂OCH₂CH₃ | 2-Cl, 3-CF₃ |
| M.843. | C(═S)NH₂ | CH₂OCH₂CH₃ | 2-Cl, 4-CF₃ |
| M.844. | C(═S)NH₂ | CH₂OCH₂CH₃ | 2-Cl, 5-CF₃ |
| M.845. | C(═S)NH₂ | CH₂OCH₂CH₃ | 3-Cl, 5-CF₃ |
| M.846. | C(═S)NH₂ | CH₂OCH₂CH₃ | 4-Cl, 3-CF₃ |
| M.847. | C(═S)NH₂ | CH₂SO₂CH₃ | — |
| M.848. | C(═S)NH₂ | CH₂SO₂CH₃ | 2-F |
| M.849. | C(═S)NH₂ | CH₂SO₂CH₃ | 3-F |
| M.850. | C(═S)NH₂ | CH₂SO₂CH₃ | 4-F |
| M.851. | C(═S)NH₂ | CH₂SO₂CH₃ | 2-Cl |
| M.852. | C(═S)NH₂ | CH₂SO₂CH₃ | 3-Cl |
| M.853. | C(═S)NH₂ | CH₂SO₂CH₃ | 4-Cl |
| M.854. | C(═S)NH₂ | CH₂SO₂CH₃ | 2-Br |
| M.855. | C(═S)NH₂ | CH₂SO₂CH₃ | 3-Br |
| M.856. | C(═S)NH₂ | CH₂SO₂CH₃ | 4-Br |
| M.857. | C(═S)NH₂ | CH₂SO₂CH₃ | 4-I |
| M.858. | C(═S)NH₂ | CH₂SO₂CH₃ | 2-CH₃ |
| M.859. | C(═S)NH₂ | CH₂SO₂CH₃ | 3-CH₃ |
| M.860. | C(═S)NH₂ | CH₂SO₂CH₃ | 4-CH₃ |
| M.861. | C(═S)NH₂ | CH₂SO₂CH₃ | 2-CF₃ |
| M.862. | C(═S)NH₂ | CH₂SO₂CH₃ | 3-CF₃ |
| M.863. | C(═S)NH₂ | CH₂SO₂CH₃ | 4-CF₃ |
| M.864. | C(═S)NH₂ | CH₂SO₂CH₃ | 2-OCF₃ |
| M.865. | C(═S)NH₂ | CH₂SO₂CH₃ | 3-OCF₃ |
| M.866. | C(═S)NH₂ | CH₂SO₂CH₃ | 4-OCF₃ |
| M.867. | C(═S)NH₂ | CH₂SO₂CH₃ | 4-OCHF₂ |
| M.868. | C(═S)NH₂ | CH₂SO₂CH₃ | 2-OCF₂O-3 |
| M.869. | C(═S)NH₂ | CH₂SO₂CH₃ | 3-OCF₂O-4 |
| M.870. | C(═S)NH₂ | CH₂SO₂CH₃ | 2-Cl, 4-O—CF₂—O-5 |
| M.871. | C(═S)NH₂ | CH₂SO₂CH₃ | 2-SCF₃ |
| M.872. | C(═S)NH₂ | CH₂SO₂CH₃ | 3-SCF₃ |
| M.873. | C(═S)NH₂ | CH₂SO₂CH₃ | 4-SCF₃ |
| M.874. | C(═S)NH₂ | CH₂SO₂CH₃ | 2-CN |
| M.875. | C(═S)NH₂ | CH₂SO₂CH₃ | 3-CN |
| M.876. | C(═S)NH₂ | CH₂SO₂CH₃ | 4-CN |
| M.877. | C(═S)NH₂ | CH₂SO₂CH₃ | 3-CN, 4-F |
| M.878. | C(═S)NH₂ | CH₂SO₂CH₃ | 3-CN, 4-Cl |
| M.879. | C(═S)NH₂ | CH₂SO₂CH₃ | 2-F, 5-CN |
| M.880. | C(═S)NH₂ | CH₂SO₂CH₃ | 2-Cl, 5-CN |
| M.881. | C(═S)NH₂ | CH₂SO₂CH₃ | 2-F, 4-CN |
| M.882. | C(═S)NH₂ | CH₂SO₂CH₃ | 2-Cl, 4-CN |
| M.883. | C(═S)NH₂ | CH₂SO₂CH₃ | 3-F, 4-CN |
| M.884. | C(═S)NH₂ | CH₂SO₂CH₃ | 3-Cl, 4-CN |
| M.885. | C(═S)NH₂ | CH₂SO₂CH₃ | 3-SO₂CH₃ |
| M.886. | C(═S)NH₂ | CH₂SO₂CH₃ | 4-SO₂CH₃ |
| M.887. | C(═S)NH₂ | CH₂SO₂CH₃ | 2-F, 4-SO₂CH₃ |
| M.888. | C(═S)NH₂ | CH₂SO₂CH₃ | 2-Cl, 4-SO₂CH₃ |
| M.889. | C(═S)NH₂ | CH₂SO₂CH₃ | 3-F, 4-SO₂CH₃ |
| M.890. | C(═S)NH₂ | CH₂SO₂CH₃ | 3-Cl, 4-SO₂CH₃ |
| M.891. | C(═S)NH₂ | CH₂SO₂CH₃ | 3-SO₂CH₃, 4-F |
| M.892. | C(═S)NH₂ | CH₂SO₂CH₃ | 3-SO₂CH₃, 4-Cl |
| M.893. | C(═S)NH₂ | CH₂SO₂CH₃ | 2-F, 5-SO₂CH₃ |
| M.894. | C(═S)NH₂ | CH₂SO₂CH₃ | 2-Cl, 5-SO₂CH₃ |
| M.895. | C(═S)NH₂ | CH₂SO₂CH₃ | 2,3-F₂ |
| M.896. | C(═S)NH₂ | CH₂SO₂CH₃ | 2,4-F₂ |
| M.897. | C(═S)NH₂ | CH₂SO₂CH₃ | 2,5-F₂ |
| M.898. | C(═S)NH₂ | CH₂SO₂CH₃ | 2,6-F₂ |
| M.899. | C(═S)NH₂ | CH₂SO₂CH₃ | 3,4-F₂ |
| M.900. | C(═S)NH₂ | CH₂SO₂CH₃ | 3,5-F₂ |
| M.901. | C(═S)NH₂ | CH₂SO₂CH₃ | 2,4-Cl₂ |
| M.902. | C(═S)NH₂ | CH₂SO₂CH₃ | 2,5-Cl₂ |
| M.903. | C(═S)NH₂ | CH₂SO₂CH₃ | 3,4-Cl₂ |
| M.904. | C(═S)NH₂ | CH₂SO₂CH₃ | 2,3,4-F₃ |
| M.905. | C(═S)NH₂ | CH₂SO₂CH₃ | 2,3,5-F₃ |
| M.906. | C(═S)NH₂ | CH₂SO₂CH₃ | 2,3,6-F₃ |
| M.907. | C(═S)NH₂ | CH₂SO₂CH₃ | 3,4,5-F₃ |
| M.908. | C(═S)NH₂ | CH₂SO₂CH₃ | 2,4,6-F₃ |
| M.909. | C(═S)NH₂ | CH₂SO₂CH₃ | 2-F, 3-Cl |
| M.910. | C(═S)NH₂ | CH₂SO₂CH₃ | 2-F, 4-Cl |
| M.911. | C(═S)NH₂ | CH₂SO₂CH₃ | 2-F, 5-Cl |
| M.912. | C(═S)NH₂ | CH₂SO₂CH₃ | 3-F, 4-Cl |
| M.913. | C(═S)NH₂ | CH₂SO₂CH₃ | 2-Cl, 4-F |
| M.914. | C(═S)NH₂ | CH₂SO₂CH₃ | 3-Cl, 4-F |
| M.915. | C(═S)NH₂ | CH₂SO₂CH₃ | 2-Cl, 4,5-F₂ |
| M.916. | C(═S)NH₂ | CH₂SO₂CH₃ | 2-Cl, 4-Br |
| M.917. | C(═S)NH₂ | CH₂SO₂CH₃ | 2-Cl, 5-Br |
| M.918. | C(═S)NH₂ | CH₂SO₂CH₃ | 3-Cl, 4-Br |
| M.919. | C(═S)NH₂ | CH₂SO₂CH₃ | 2-F, 4-Br |
| M.920. | C(═S)NH₂ | CH₂SO₂CH₃ | 2-F, 5-Br |
| M.921. | C(═S)NH₂ | CH₂SO₂CH₃ | 3-F, 4-Br |
| M.922. | C(═S)NH₂ | CH₂SO₂CH₃ | 2-Br, 4-F |
| M.923. | C(═S)NH₂ | CH₂SO₂CH₃ | 3-Br, 4-F |
| M.924. | C(═S)NH₂ | CH₂SO₂CH₃ | 2-Br, 5-Cl |
| M.925. | C(═S)NH₂ | CH₂SO₂CH₃ | 3-Br, 4-Cl |
| M.926. | C(═S)NH₂ | CH₂SO₂CH₃ | 2-Br, 4,5-F₂ |
| M.927. | C(═S)NH₂ | CH₂SO₂CH₃ | 3- ═N—O—N═ -4 |
| M.928. | C(═S)NH₂ | CH₂SO₂CH₃ | 4-CHF₂ |
| M.929. | C(═S)NH₂ | CH₂SO₂CH₃ | 2-F, 3-CF₃ |
| M.930. | C(═S)NH₂ | CH₂SO₂CH₃ | 2-F, 4-CF₃ |
| M.931. | C(═S)NH₂ | CH₂SO₂CH₃ | 2-F, 5-CF₃ |
| M.932. | C(═S)NH₂ | CH₂SO₂CH₃ | 3-F, 4-CF₃ |
| M.933. | C(═S)NH₂ | CH₂SO₂CH₃ | 3-F, 5-CF₃ |
| M.934. | C(═S)NH₂ | CH₂SO₂CH₃ | 4-F, 3-CF₃ |
| M.935. | C(═S)NH₂ | CH₂SO₂CH₃ | 3,4-F₂, 5-CF₃ |
| M.936. | C(═S)NH₂ | CH₂SO₂CH₃ | 2-Cl, 3-CF₃ |
| M.937. | C(═S)NH₂ | CH₂SO₂CH₃ | 2-Cl, 4-CF₃ |
| M.938. | C(═S)NH₂ | CH₂SO₂CH₃ | 2-Cl, 5-CF₃ |
| M.939. | C(═S)NH₂ | CH₂SO₂CH₃ | 3-Cl, 5-CF₃ |
| M.940. | C(═S)NH₂ | CH₂SO₂CH₃ | 4-Cl, 3-CF₃ |
| M.941. | C(═S)NH₂ | CH₂CN | — |
| M.942. | C(═S)NH₂ | CH₂CN | 2-F |
| M.943. | C(═S)NH₂ | CH₂CN | 3-F |
| M.944. | C(═S)NH₂ | CH₂CN | 4-F |
| M.945. | C(═S)NH₂ | CH₂CN | 2-Cl |
| M.946. | C(═S)NH₂ | CH₂CN | 3-Cl |
| M.947. | C(═S)NH₂ | CH₂CN | 4-Cl |
| M.948. | C(═S)NH₂ | CH₂CN | 2-Br |
| M.949. | C(═S)NH₂ | CH₂CN | 3-Br |
| M.950. | C(═S)NH₂ | CH₂CN | 4-Br |
| M.951. | C(═S)NH₂ | CH₂CN | 4-I |
| M.952. | C(═S)NH₂ | CH₂CN | 2-CH₃ |
| M.953. | C(═S)NH₂ | CH₂CN | 3-CH₃ |
| M.954. | C(═S)NH₂ | CH₂CN | 4-CH₃ |
| M.955. | C(═S)NH₂ | CH₂CN | 2-CF₃ |
| M.956. | C(═S)NH₂ | CH₂CN | 3-CF₃ |
| M.957. | C(═S)NH₂ | CH₂CN | 4-CF₃ |
| M.958. | C(═S)NH₂ | CH₂CN | 2-OCF₃ |
| M.959. | C(═S)NH₂ | CH₂CN | 3-OCF₃ |
| M.960. | C(═S)NH₂ | CH₂CN | 4-OCF₃ |
| M.961. | C(═S)NH₂ | CH₂CN | 4-OCHF₂ |
| M.962. | C(═S)NH₂ | CH₂CN | 2-OCF₂O-3 |
| M.963. | C(═S)NH₂ | CH₂CN | 3-OCF₂O-4 |
| M.964. | C(═S)NH₂ | CH₂CN | 2-Cl, 4-O—CF₂—O-5 |
| M.965. | C(═S)NH₂ | CH₂CN | 2-SCF₃ |
| M.966. | C(═S)NH₂ | CH₂CN | 3-SCF₃ |
| M.967. | C(═S)NH₂ | CH₂CN | 4-SCF₃ |
| M.968. | C(═S)NH₂ | CH₂CN | 2-CN |
| M.969. | C(═S)NH₂ | CH₂CN | 3-CN |
| M.970. | C(═S)NH₂ | CH₂CN | 4-CN |
| M.971. | C(═S)NH₂ | CH₂CN | 3-CN, 4-F |
| M.972. | C(═S)NH₂ | CH₂CN | 3-CN, 4-Cl |
| M.973. | C(═S)NH₂ | CH₂CN | 2-F, 5-CN |
| M.974. | C(═S)NH₂ | CH₂CN | 2-Cl, 5-CN |
| M.975. | C(═S)NH₂ | CH₂CN | 2-F, 4-CN |
| M.976. | C(═S)NH₂ | CH₂CN | 2-Cl, 4-CN |
| M.977. | C(═S)NH₂ | CH₂CN | 3-F, 4-CN |
| M.978. | C(═S)NH₂ | CH₂CN | 3-Cl, 4-CN |
| M.979. | C(═S)NH₂ | CH₂CN | 3-SO₂CH₃ |
| M.980. | C(═S)NH₂ | CH₂CN | 4-SO₂CH₃ |
| M.981. | C(═S)NH₂ | CH₂CN | 2-F, 4-SO₂CH₃ |
| M.982. | C(═S)NH₂ | CH₂CN | 2-Cl, 4-SO₂CH₃ |
| M.983. | C(═S)NH₂ | CH₂CN | 3-F, 4-SO₂CH₃ |
| M.984. | C(═S)NH₂ | CH₂CN | 3-Cl, 4-SO₂CH₃ |
| M.985. | C(═S)NH₂ | CH₂CN | 3-SO₂CH₃, 4-F |
| M.986. | C(═S)NH₂ | CH₂CN | 3-SO₂CH₃, 4-Cl |
| M.987. | C(═S)NH₂ | CH₂CN | 2-F, 5-SO₂CH₃ |
| M.988. | C(═S)NH₂ | CH₂CN | 2-Cl, 5-SO₂CH₃ |
| M.989. | C(═S)NH₂ | CH₂CN | 2,3-F₂ |
| M.990. | C(═S)NH₂ | CH₂CN | 2,4-F₂ |
| M.991. | C(═S)NH₂ | CH₂CN | 2,5-F₂ |
| M.992. | C(═S)NH₂ | CH₂CN | 2,6-F₂ |
| M.993. | C(═S)NH₂ | CH₂CN | 3,4-F₂ |
| M.994. | C(═S)NH₂ | CH₂CN | 3,5-F₂ |
| M.995. | C(═S)NH₂ | CH₂CN | 2,4-Cl₂ |
| M.996. | C(═S)NH₂ | CH₂CN | 2,5-Cl₂ |
| M.997. | C(═S)NH₂ | CH₂CN | 3,4-Cl₂ |
| M.998. | C(═S)NH₂ | CH₂CN | 2,3,4-F₃ |
| M.999. | C(═S)NH₂ | CH₂CN | 2,3,5-F₃ |
| M.1000. | C(═S)NH₂ | CH₂CN | 2,3,6-F₃ |
| M.1001. | C(═S)NH₂ | CH₂CN | 3,4,5-F₃ |
| M.1002. | C(═S)NH₂ | CH₂CN | 2,4,6-F₃ |
| M.1003. | C(═S)NH₂ | CH₂CN | 2-F, 3-Cl |
| M.1004. | C(═S)NH₂ | CH₂CN | 2-F, 4-Cl |
| M.1005. | C(═S)NH₂ | CH₂CN | 2-F, 5-Cl |
| M.1006. | C(═S)NH₂ | CH₂CN | 3-F, 4-Cl |
| M.1007. | C(═S)NH₂ | CH₂CN | 2-Cl, 4-F |
| M.1008. | C(═S)NH₂ | CH₂CN | 3-Cl, 4-F |
| M.1009. | C(═S)NH₂ | CH₂CN | 2-Cl, 4,5-F₂ |
| M.1010. | C(═S)NH₂ | CH₂CN | 2-Cl, 4-Br |
| M.1011. | C(═S)NH₂ | CH₂CN | 2-Cl, 5-Br |
| M.1012. | C(═S)NH₂ | CH₂CN | 3-Cl, 4-Br |
| M.1013. | C(═S)NH₂ | CH₂CN | 2-F, 4-Br |
| M.1014. | C(═S)NH₂ | CH₂CN | 2-F, 5-Br |
| M.1015. | C(═S)NH₂ | CH₂CN | 3-F, 4-Br |
| M.1016. | C(═S)NH₂ | CH₂CN | 2-Br, 4-F |
| M.1017. | C(═S)NH₂ | CH₂CN | 3-Br, 4-F |
| M.1018. | C(═S)NH₂ | CH₂CN | 2-Br, 5-Cl |
| M.1019. | C(═S)NH₂ | CH₂CN | 3-Br, 4-Cl |
| M.1020. | C(═S)NH₂ | CH₂CN | 2-Br, 4,5-F₂ |
| M.1021. | C(═S)NH₂ | CH₂CN | 3- ═N—O—N═ -4 |
| M.1022. | C(═S)NH₂ | CH₂CN | 4-CHF₂ |
| M.1023. | C(═S)NH₂ | CH₂CN | 2-F, 3-CF₃ |
| M.1024. | C(═S)NH₂ | CH₂CN | 2-F, 4-CF₃ |
| M.1025. | C(═S)NH₂ | CH₂CN | 2-F, 5-CF₃ |
| M.1026. | C(═S)NH₂ | CH₂CN | 3-F, 4-CF₃ |
| M.1027. | C(═S)NH₂ | CH₂CN | 3-F, 5-CF₃ |
| M.1028. | C(═S)NH₂ | CH₂CN | 4-F, 3-CF₃ |
| M.1029. | C(═S)NH₂ | CH₂CN | 3,4-F₂, 5-CF₃ |
| M.1030. | C(═S)NH₂ | CH₂CN | 2-Cl, 3-CF₃ |
| M.1031. | C(═S)NH₂ | CH₂CN | 2-Cl, 4-CF₃ |
| M.1032. | C(═S)NH₂ | CH₂CN | 2-Cl, 5-CF₃ |
| M.1033. | C(═S)NH₂ | CH₂CN | 3-Cl, 5-CF₃ |
| M.1034. | C(═S)NH₂ | CH₂CN | 4-Cl, 3-CF₃ |
| M.1035. | C(═S)NH₂ | C(CH₃)₂CN | — |
| M.1036. | C(═S)NH₂ | C(CH₃)₂CN | 2-F |
| M.1037. | C(═S)NH₂ | C(CH₃)₂CN | 3-F |
| M.1038. | C(═S)NH₂ | C(CH₃)₂CN | 4-F |
| M.1039. | C(═S)NH₂ | C(CH₃)₂CN | 2-Cl |
| M.1040. | C(═S)NH₂ | C(CH₃)₂CN | 3-Cl |
| M.1041. | C(═S)NH₂ | C(CH₃)₂CN | 4-Cl |
| M.1042. | C(═S)NH₂ | C(CH₃)₂CN | 2-Br |
| M.1043. | C(═S)NH₂ | C(CH₃)₂CN | 3-Br |
| M.1044. | C(═S)NH₂ | C(CH₃)₂CN | 4-Br |
| M.1045. | C(═S)NH₂ | C(CH₃)₂CN | 4-I |
| M.1046. | C(═S)NH₂ | C(CH₃)₂CN | 2-CH₃ |
| M.1047. | C(═S)NH₂ | C(CH₃)₂CN | 3-CH₃ |
| M.1048. | C(═S)NH₂ | C(CH₃)₂CN | 4-CH₃ |
| M.1049. | C(═S)NH₂ | C(CH₃)₂CN | 2-CF₃ |
| M.1050. | C(═S)NH₂ | C(CH₃)₂CN | 3-CF₃ |
| M.1051. | C(═S)NH₂ | C(CH₃)₂CN | 4-CF₃ |
| M.1052. | C(═S)NH₂ | C(CH₃)₂CN | 2-OCF₃ |
| M.1053. | C(═S)NH₂ | C(CH₃)₂CN | 3-OCF₃ |
| M.1054. | C(═S)NH₂ | C(CH₃)₂CN | 4-OCF₃ |
| M.1055. | C(═S)NH₂ | C(CH₃)₂CN | 4-OCHF₂ |
| M.1056. | C(═S)NH₂ | C(CH₃)₂CN | 2-OCF₂O-3 |
| M.1057. | C(═S)NH₂ | C(CH₃)₂CN | 3-OCF₂O-4 |
| M.1058. | C(═S)NH₂ | C(CH₃)₂CN | 2-Cl, 4-O—CF₂—O-5 |
| M.1059. | C(═S)NH₂ | C(CH₃)₂CN | 2-SCF₃ |
| M.1060. | C(═S)NH₂ | C(CH₃)₂CN | 3-SCF₃ |
| M.1061. | C(═S)NH₂ | C(CH₃)₂CN | 4-SCF₃ |
| M.1062. | C(═S)NH₂ | C(CH₃)₂CN | 2-CN |
| M.1063. | C(═S)NH₂ | C(CH₃)₂CN | 3-CN |
| M.1064. | C(═S)NH₂ | C(CH₃)₂CN | 4-CN |
| M.1065. | C(═S)NH₂ | C(CH₃)₂CN | 3-CN, 4-F |
| M.1066. | C(═S)NH₂ | C(CH₃)₂CN | 3-CN, 4-Cl |
| M.1067. | C(═S)NH₂ | C(CH₃)₂CN | 2-F, 5-CN |
| M.1068. | C(═S)NH₂ | C(CH₃)₂CN | 2-Cl, 5-CN |
| M.1069. | C(═S)NH₂ | C(CH₃)₂CN | 2-F, 4-CN |
| M.1070. | C(═S)NH₂ | C(CH₃)₂CN | 2-Cl, 4-CN |
| M.1071. | C(═S)NH₂ | C(CH₃)₂CN | 3-F, 4-CN |
| M.1072. | C(═S)NH₂ | C(CH₃)₂CN | 3-Cl, 4-CN |
| M.1073. | C(═S)NH₂ | C(CH₃)₂CN | 3-SO₂CH₃ |
| M.1074. | C(═S)NH₂ | C(CH₃)₂CN | 4-SO₂CH₃ |
| M.1075. | C(═S)NH₂ | C(CH₃)₂CN | 2-F, 4-SO₂CH₃ |
| M.1076. | C(═S)NH₂ | C(CH₃)₂CN | 2-Cl, 4-SO₂CH₃ |
| M.1077. | C(═S)NH₂ | C(CH₃)₂CN | 3-F, 4-SO₂CH₃ |
| M.1078. | C(═S)NH₂ | C(CH₃)₂CN | 3-Cl, 4-SO₂CH₃ |
| M.1079. | C(═S)NH₂ | C(CH₃)₂CN | 3-SO₂CH₃, 4-F |
| M.1080. | C(═S)NH₂ | C(CH₃)₂CN | 3-SO₂CH₃, 4-Cl |
| M.1081. | C(═S)NH₂ | C(CH₃)₂CN | 2-F, 5-SO₂CH₃ |
| M.1082. | C(═S)NH₂ | C(CH₃)₂CN | 2-Cl, 5-SO₂CH₃ |
| M.1083. | C(═S)NH₂ | C(CH₃)₂CN | 2,3-F₂ |
| M.1084. | C(═S)NH₂ | C(CH₃)₂CN | 2,4-F₂ |
| M.1085. | C(═S)NH₂ | C(CH₃)₂CN | 2,5-F₂ |
| M.1086. | C(═S)NH₂ | C(CH₃)₂CN | 2,6-F₂ |
| M.1087. | C(═S)NH₂ | C(CH₃)₂CN | 3,4-F₂ |
| M.1088. | C(═S)NH₂ | C(CH₃)₂CN | 3,5-F₂ |
| M.1089. | C(═S)NH₂ | C(CH₃)₂CN | 2,4-Cl₂ |
| M.1090. | C(═S)NH₂ | C(CH₃)₂CN | 2,5-Cl₂ |
| M.1091. | C(═S)NH₂ | C(CH₃)₂CN | 3,4-Cl₂ |
| M.1092. | C(═S)NH₂ | C(CH₃)₂CN | 2,3,4-F₃ |
| M.1093. | C(═S)NH₂ | C(CH₃)₂CN | 2,3,5-F₃ |
| M.1094. | C(═S)NH₂ | C(CH₃)₂CN | 2,3,6-F₃ |
| M.1095. | C(═S)NH₂ | C(CH₃)₂CN | 3,4,5-F₃ |
| M.1096. | C(═S)NH₂ | C(CH₃)₂CN | 2,4,6-F₃ |
| M.1097. | C(═S)NH₂ | C(CH₃)₂CN | 2-F, 3-Cl |
| M.1098. | C(═S)NH₂ | C(CH₃)₂CN | 2-F, 4-Cl |
| M.1099. | C(═S)NH₂ | C(CH₃)₂CN | 2-F, 5-Cl |
| M.1100. | C(═S)NH₂ | C(CH₃)₂CN | 3-F, 4-Cl |
| M.1101. | C(═S)NH₂ | C(CH₃)₂CN | 2-Cl, 4-F |
| M.1102. | C(═S)NH₂ | C(CH₃)₂CN | 3-Cl, 4-F |
| M.1103. | C(═S)NH₂ | C(CH₃)₂CN | 2-Cl, 4,5-F₂ |
| M.1104. | C(═S)NH₂ | C(CH₃)₂CN | 2-Cl, 4-Br |
| M.1105. | C(═S)NH₂ | C(CH₃)₂CN | 2-Cl, 5-Br |
| M.1106. | C(═S)NH₂ | C(CH₃)₂CN | 3-Cl, 4-Br |
| M.1107. | C(═S)NH₂ | C(CH₃)₂CN | 2-F, 4-Br |
| M.1108. | C(═S)NH₂ | C(CH₃)₂CN | 2-F, 5-Br |
| M.1109. | C(═S)NH₂ | C(CH₃)₂CN | 3-F, 4-Br |
| M.1110. | C(═S)NH₂ | C(CH₃)₂CN | 2-Br, 4-F |
| M.1111. | C(═S)NH₂ | C(CH₃)₂CN | 3-Br, 4-F |
| M.1112. | C(═S)NH₂ | C(CH₃)₂CN | 2-Br, 5-Cl |
| M.1113. | C(═S)NH₂ | C(CH₃)₂CN | 3-Br, 4-Cl |
| M.1114. | C(═S)NH₂ | C(CH₃)₂CN | 2-Br, 4,5-F₂ |
| M.1115. | C(═S)NH₂ | C(CH₃)₂CN | 3- ═N—O—N═ -4 |
| M.1116. | C(═S)NH₂ | C(CH₃)₂CN | 4-CHF₂ |
| M.1117. | C(═S)NH₂ | C(CH₃)₂CN | 2-F, 3-CF₃ |
| M.1118. | C(═S)NH₂ | C(CH₃)₂CN | 2-F, 4-CF₃ |
| M.1119. | C(═S)NH₂ | C(CH₃)₂CN | 2-F, 5-CF₃ |
| M.1120. | C(═S)NH₂ | C(CH₃)₂CN | 3-F, 4-CF₃ |
| M.1121. | C(═S)NH₂ | C(CH₃)₂CN | 3-F, 5-CF₃ |
| M.1122. | C(═S)NH₂ | C(CH₃)₂CN | 4-F, 3-CF₃ |
| M.1123. | C(═S)NH₂ | C(CH₃)₂CN | 3,4-F₂, 5-CF₃ |
| M.1124. | C(═S)NH₂ | C(CH₃)₂CN | 2-Cl, 3-CF₃ |
| M.1125. | C(═S)NH₂ | C(CH₃)₂CN | 2-Cl, 4-CF₃ |
| M.1126. | C(═S)NH₂ | C(CH₃)₂CN | 2-Cl, 5-CF₃ |
| M.1127. | C(═S)NH₂ | C(CH₃)₂CN | 3-Cl, 5-CF₃ |
| M.1128. | C(═S)NH₂ | C(CH₃)₂CN | 4-Cl, 3-CF₃ |
| M.1129. | C(═S)NH₂ | 1-cyanocyclopropyl | — |
| M.1130. | C(═S)NH₂ | 1-cyanocyclopropyl | 2-F |
| M.1131. | C(═S)NH₂ | 1-cyanocyclopropyl | 3-F |
| M.1132. | C(═S)NH₂ | 1-cyanocyclopropyl | 4-F |
| M.1133. | C(═S)NH₂ | 1-cyanocyclopropyl | 2-Cl |
| M.1134. | C(═S)NH₂ | 1-cyanocyclopropyl | 3-Cl |
| M.1135. | C(═S)NH₂ | 1-cyanocyclopropyl | 4-Cl |
| M.1136. | C(═S)NH₂ | 1-cyanocyclopropyl | 2-Br |
| M.1137. | C(═S)NH₂ | 1-cyanocyclopropyl | 3-Br |
| M.1138. | C(═S)NH₂ | 1-cyanocyclopropyl | 4-Br |
| M.1139. | C(═S)NH₂ | 1-cyanocyclopropyl | 4-I |
| M.1140. | C(═S)NH₂ | 1-cyanocyclopropyl | 2-CH₃ |
| M.1141. | C(═S)NH₂ | 1-cyanocyclopropyl | 3-CH₃ |
| M.1142. | C(═S)NH₂ | 1-cyanocyclopropyl | 4-CH₃ |
| M.1143. | C(═S)NH₂ | 1-cyanocyclopropyl | 2-CF₃ |
| M.1144. | C(═S)NH₂ | 1-cyanocyclopropyl | 3-CF₃ |
| M.1145. | C(═S)NH₂ | 1-cyanocyclopropyl | 4-CF₃ |
| M.1146. | C(═S)NH₂ | 1-cyanocyclopropyl | 2-OCF₃ |
| M.1147. | C(═S)NH₂ | 1-cyanocyclopropyl | 3-OCF₃ |
| M.1148. | C(═S)NH₂ | 1-cyanocyclopropyl | 4-OCF₃ |
| M.1149. | C(═S)NH₂ | 1-cyanocyclopropyl | 4-OCHF₂ |
| M.1150. | C(═S)NH₂ | 1-cyanocyclopropyl | 2-OCF₂O-3 |
| M.1151. | C(═S)NH₂ | 1-cyanocyclopropyl | 3-OCF₂O-4 |
| M.1152. | C(═S)NH₂ | 1-cyanocyclopropyl | 2-Cl, 4-O—CF₂—O-5 |
| M.1153. | C(═S)NH₂ | 1-cyanocyclopropyl | 2-SCF₃ |
| M.1154. | C(═S)NH₂ | 1-cyanocyclopropyl | 3-SCF₃ |
| M.1155. | C(═S)NH₂ | 1-cyanocyclopropyl | 4-SCF₃ |
| M.1156. | C(═S)NH₂ | 1-cyanocyclopropyl | 2-CN |
| M.1157. | C(═S)NH₂ | 1-cyanocyclopropyl | 3-CN |
| M.1158. | C(═S)NH₂ | 1-cyanocyclopropyl | 4-CN |
| M.1159. | C(═S)NH₂ | 1-cyanocyclopropyl | 3-CN, 4-F |
| M.1160. | C(═S)NH₂ | 1-cyanocyclopropyl | 3-CN, 4-Cl |
| M.1161. | C(═S)NH₂ | 1-cyanocyclopropyl | 2-F, 5-CN |
| M.1162. | C(═S)NH₂ | 1-cyanocyclopropyl | 2-Cl, 5-CN |
| M.1163. | C(═S)NH₂ | 1-cyanocyclopropyl | 2-F, 4-CN |
| M.1164. | C(═S)NH₂ | 1-cyanocyclopropyl | 2-Cl, 4-CN |
| M.1165. | C(═S)NH₂ | 1-cyanocyclopropyl | 3-F, 4-CN |
| M.1166. | C(═S)NH₂ | 1-cyanocyclopropyl | 3-Cl, 4-CN |
| M.1167. | C(═S)NH₂ | 1-cyanocyclopropyl | 3-SO₂CH₃ |
| M.1168. | C(═S)NH₂ | 1-cyanocyclopropyl | 4-SO₂CH₃ |
| M.1169. | C(═S)NH₂ | 1-cyanocyclopropyl | 2-F, 4-SO₂CH₃ |
| M.1170. | C(═S)NH₂ | 1-cyanocyclopropyl | 2-Cl, 4-SO₂CH₃ |
| M.1171. | C(═S)NH₂ | 1-cyanocyclopropyl | 3-F, 4-SO₂CH₃ |
| M.1172. | C(═S)NH₂ | 1-cyanocyclopropyl | 3-Cl, 4-SO₂CH₃ |
| M.1173. | C(═S)NH₂ | 1-cyanocyclopropyl | 3-SO₂CH₃, 4-F |
| M.1174. | C(═S)NH₂ | 1-cyanocyclopropyl | 3-SO₂CH₃, 4-Cl |
| M.1175. | C(═S)NH₂ | 1-cyanocyclopropyl | 2-F, 5-SO₂CH₃ |
| M.1176. | C(═S)NH₂ | 1-cyanocyclopropyl | 2-Cl, 5-SO₂CH₃ |
| M.1177. | C(═S)NH₂ | 1-cyanocyclopropyl | 2,3-F₂ |
| M.1178. | C(═S)NH₂ | 1-cyanocyclopropyl | 2,4-F₂ |
| M.1179. | C(═S)NH₂ | 1-cyanocyclopropyl | 2,5-F₂ |
| M.1180. | C(═S)NH₂ | 1-cyanocyclopropyl | 2,6-F₂ |
| M.1181. | C(═S)NH₂ | 1-cyanocyclopropyl | 3,4-F₂ |
| M.1182. | C(═S)NH₂ | 1-cyanocyclopropyl | 3,5-F₂ |
| M.1183. | C(═S)NH₂ | 1-cyanocyclopropyl | 2,4-Cl₂ |
| M.1184. | C(═S)NH₂ | 1-cyanocyclopropyl | 2,5-Cl₂ |
| M.1185. | C(═S)NH₂ | 1-cyanocyclopropyl | 3,4-Cl₂ |
| M.1186. | C(═S)NH₂ | 1-cyanocyclopropyl | 2,3,4-F₃ |
| M.1187. | C(═S)NH₂ | 1-cyanocyclopropyl | 2,3,5-F₃ |
| M.1188. | C(═S)NH₂ | 1-cyanocyclopropyl | 2,3,6-F₃ |
| M.1189. | C(═S)NH₂ | 1-cyanocyclopropyl | 3,4,5-F₃ |
| M.1190. | C(═S)NH₂ | 1-cyanocyclopropyl | 2,4,6-F₃ |
| M.1191. | C(═S)NH₂ | 1-cyanocyclopropyl | 2-F, 3-Cl |
| M.1192. | C(═S)NH₂ | 1-cyanocyclopropyl | 2-F, 4-Cl |
| M.1193. | C(═S)NH₂ | 1-cyanocyclopropyl | 2-F, 5-Cl |
| M.1194. | C(═S)NH₂ | 1-cyanocyclopropyl | 3-F, 4-Cl |
| M.1195. | C(═S)NH₂ | 1-cyanocyclopropyl | 2-Cl, 4-F |
| M.1196. | C(═S)NH₂ | 1-cyanocyclopropyl | 3-Cl, 4-F |
| M.1197. | C(═S)NH₂ | 1-cyanocyclopropyl | 2-Cl, 4,5-F₂ |
| M.1198. | C(═S)NH₂ | 1-cyanocyclopropyl | 2-Cl, 4-Br |
| M.1199. | C(═S)NH₂ | 1-cyanocyclopropyl | 2-Cl, 5-Br |
| M.1200. | C(═S)NH₂ | 1-cyanocyclopropyl | 3-Cl, 4-Br |
| M.1201. | C(═S)NH₂ | 1-cyanocyclopropyl | 2-F, 4-Br |
| M.1202. | C(═S)NH₂ | 1-cyanocyclopropyl | 2-F, 5-Br |
| M.1203. | C(═S)NH₂ | 1-cyanocyclopropyl | 3-F, 4-Br |
| M.1204. | C(═S)NH₂ | 1-cyanocyclopropyl | 2-Br, 4-F |
| M.1205. | C(═S)NH₂ | 1-cyanocyclopropyl | 3-Br, 4-F |
| M.1206. | C(═S)NH₂ | 1-cyanocyclopropyl | 2-Br, 5-Cl |
| M.1207. | C(═S)NH₂ | 1-cyanocyclopropyl | 3-Br, 4-Cl |
| M.1208. | C(═S)NH₂ | 1-cyanocyclopropyl | 2-Br, 4,5-F₂ |
| M.1209. | C(═S)NH₂ | 1-cyanocyclopropyl | 3- ═N—O—N═ -4 |
| M.1210. | C(═S)NH₂ | 1-cyanocyclopropyl | 4-CHF₂ |
| M.1211. | C(═S)NH₂ | 1-cyanocyclopropyl | 2-F, 3-CF₃ |
| M.1212. | C(═S)NH₂ | 1-cyanocyclopropyl | 2-F, 4-CF₃ |
| M.1213. | C(═S)NH₂ | 1-cyanocyclopropyl | 2-F, 5-CF₃ |
| M.1214. | C(═S)NH₂ | 1-cyanocyclopropyl | 3-F, 4-CF₃ |
| M.1215. | C(═S)NH₂ | 1-cyanocyclopropyl | 3-F, 5-CF₃ |
| M.1216. | C(═S)NH₂ | 1-cyanocyclopropyl | 4-F, 3-CF₃ |
| M.1217. | C(═S)NH₂ | 1-cyanocyclopropyl | 3,4-F₂, 5-CF₃ |
| M.1218. | C(═S)NH₂ | 1-cyanocyclopropyl | 2-Cl, 3-CF₃ |
| M.1219. | C(═S)NH₂ | 1-cyanocyclopropyl | 2-Cl, 4-CF₃ |
| M.1220. | C(═S)NH₂ | 1-cyanocyclopropyl | 2-Cl, 5-CF₃ |
| M.1221. | C(═S)NH₂ | 1-cyanocyclopropyl | 3-Cl, 5-CF₃ |
| M.1222. | C(═S)NH₂ | 1-cyanocyclopropyl | 4-Cl, 3-CF₃ |
| M.1223. | C(═S)NH₂ | 1-methylsulfonylcyclopropyl | — |
| M.1224. | C(═S)NH₂ | 1-methylsulfonylcyclopropyl | 2-F |
| M.1225. | C(═S)NH₂ | 1-methylsulfonylcyclopropyl | 3-F |
| M.1226. | C(═S)NH₂ | 1-methylsulfonylcyclopropyl | 4-F |
| M.1227. | C(═S)NH₂ | 1-methylsulfonylcyclopropyl | 2-Cl |
| M.1228. | C(═S)NH₂ | 1-methylsulfonylcyclopropyl | 3-Cl |
| M.1229. | C(═S)NH₂ | 1-methylsulfonylcyclopropyl | 4-Cl |
| M.1230. | C(═S)NH₂ | 1-methylsulfonylcyclopropyl | 2-Br |
| M.1231. | C(═S)NH₂ | 1-methylsulfonylcyclopropyl | 3-Br |
| M.1232. | C(═S)NH₂ | 1-methylsulfonylcyclopropyl | 4-Br |
| M.1233. | C(═S)NH₂ | 1-methylsulfonylcyclopropyl | 4-I |
| M.1234. | C(═S)NH₂ | 1-methylsulfonylcyclopropyl | 2-CH₃ |
| M.1235. | C(═S)NH₂ | 1-methylsulfonylcyclopropyl | 3-CH₃ |
| M.1236. | C(═S)NH₂ | 1-methylsulfonylcyclopropyl | 4-CH₃ |
| M.1237. | C(═S)NH₂ | 1-methylsulfonylcyclopropyl | 2-CF₃ |
| M.1238. | C(═S)NH₂ | 1-methylsulfonylcyclopropyl | 3-CF₃ |
| M.1239. | C(═S)NH₂ | 1-methylsulfonylcyclopropyl | 4-CF₃ |
| M.1240. | C(═S)NH₂ | 1-methylsulfonylcyclopropyl | 2-OCF₃ |
| M.1241. | C(═S)NH₂ | 1-methylsulfonylcyclopropyl | 3-OCF₃ |
| M.1242. | C(═S)NH₂ | 1-methylsulfonylcyclopropyl | 4-OCF₃ |
| M.1243. | C(═S)NH₂ | 1-methylsulfonylcyclopropyl | 4-OCHF₂ |
| M.1244. | C(═S)NH₂ | 1-methylsulfonylcyclopropyl | 2-OCF₂O-3 |
| M.1245. | C(═S)NH₂ | 1-methylsulfonylcyclopropyl | 3-OCF₂O-4 |
| M.1246. | C(═S)NH₂ | 1-methylsulfonylcyclopropyl | 2-Cl, 4-O—CF₂—O-5 |
| M.1247. | C(═S)NH₂ | 1-methylsulfonylcyclopropyl | 2-SCF₃ |
| M.1248. | C(═S)NH₂ | 1-methylsulfonylcyclopropyl | 3-SCF₃ |
| M.1249. | C(═S)NH₂ | 1-methylsulfonylcyclopropyl | 4-SCF₃ |
| M.1250. | C(═S)NH₂ | 1-methylsulfonylcyclopropyl | 2-CN |
| M.1251. | C(═S)NH₂ | 1-methylsulfonylcyclopropyl | 3-CN |
| M.1252. | C(═S)NH₂ | 1-methylsulfonylcyclopropyl | 4-CN |
| M.1253. | C(═S)NH₂ | 1-methylsulfonylcyclopropyl | 3-CN, 4-F |
| M.1254. | C(═S)NH₂ | 1-methylsulfonylcyclopropyl | 3-CN, 4-Cl |
| M.1255. | C(═S)NH₂ | 1-methylsulfonylcyclopropyl | 2-F, 5-CN |
| M.1256. | C(═S)NH₂ | 1-methylsulfonylcyclopropyl | 2-Cl, 5-CN |
| M.1257. | C(═S)NH₂ | 1-methylsulfonylcyclopropyl | 2-F, 4-CN |
| M.1258. | C(═S)NH₂ | 1-methylsulfonylcyclopropyl | 2-Cl, 4-CN |
| M.1259. | C(═S)NH₂ | 1-methylsulfonylcyclopropyl | 3-F, 4-CN |
| M.1260. | C(═S)NH₂ | 1-methylsulfonylcyclopropyl | 3-Cl, 4-CN |
| M.1261. | C(═S)NH₂ | 1-methylsulfonylcyclopropyl | 3-SO₂CH₃ |
| M.1262. | C(═S)NH₂ | 1-methylsulfonylcyclopropyl | 4-SO₂CH₃ |
| M.1263. | C(═S)NH₂ | 1-methylsulfonylcyclopropyl | 2-F, 4-SO₂CH₃ |
| M.1264. | C(═S)NH₂ | 1-methylsulfonylcyclopropyl | 2-Cl, 4-SO₂CH₃ |
| M.1265. | C(═S)NH₂ | 1-methylsulfonylcyclopropyl | 3-F, 4-SO₂CH₃ |
| M.1266. | C(═S)NH₂ | 1-methylsulfonylcyclopropyl | 3-Cl, 4-SO₂CH₃ |
| M.1267. | C(═S)NH₂ | 1-methylsulfonylcyclopropyl | 3-SO₂CH₃, 4-F |
| M.1268. | C(═S)NH₂ | 1-methylsulfonylcyclopropyl | 3-SO₂CH₃, 4-Cl |
| M.1269. | C(═S)NH₂ | 1-methylsulfonylcyclopropyl | 2-F, 5-SO₂CH₃ |
| M.1270. | C(═S)NH₂ | 1-methylsulfonylcyclopropyl | 2-Cl, 5-SO₂CH₃ |
| M.1271. | C(═S)NH₂ | 1-methylsulfonylcyclopropyl | 2,3-F₂ |
| M.1272. | C(═S)NH₂ | 1-methylsulfonylcyclopropyl | 2,4-F₂ |
| M.1273. | C(═S)NH₂ | 1-methylsulfonylcyclopropyl | 2,5-F₂ |
| M.1274. | C(═S)NH₂ | 1-methylsulfonylcyclopropyl | 2,6-F₂ |
| M.1275. | C(═S)NH₂ | 1-methylsulfonylcyclopropyl | 3,4-F₂ |
| M.1276. | C(═S)NH₂ | 1-methylsulfonylcyclopropyl | 3,5-F₂ |
| M.1277. | C(═S)NH₂ | 1-methylsulfonylcyclopropyl | 2,4-Cl₂ |
| M.1278. | C(═S)NH₂ | 1-methylsulfonylcyclopropyl | 2,5-Cl₂ |
| M.1279. | C(═S)NH₂ | 1-methylsulfonylcyclopropyl | 3,4-Cl₂ |
| M.1280. | C(═S)NH₂ | 1-methylsulfonylcyclopropyl | 2,3,4-F₃ |
| M.1281. | C(═S)NH₂ | 1-methylsulfonylcyclopropyl | 2,3,5-F₃ |
| M.1282. | C(═S)NH₂ | 1-methylsulfonylcyclopropyl | 2,3,6-F₃ |
| M.1283. | C(═S)NH₂ | 1-methylsulfonylcyclopropyl | 3,4,5-F₃ |
| M.1284. | C(═S)NH₂ | 1-methylsulfonylcyclopropyl | 2,4,6-F₃ |
| M.1285. | C(═S)NH₂ | 1-methylsulfonylcyclopropyl | 2-F, 3-Cl |
| M.1286. | C(═S)NH₂ | 1-methylsulfonylcyclopropyl | 2-F, 4-Cl |
| M.1287. | C(═S)NH₂ | 1-methylsulfonylcyclopropyl | 2-F, 5-Cl |
| M.1288. | C(═S)NH₂ | 1-methylsulfonylcyclopropyl | 3-F, 4-Cl |
| M.1289. | C(═S)NH₂ | 1-methylsulfonylcyclopropyl | 2-Cl, 4-F |
| M.1290. | C(═S)NH₂ | 1-methylsulfonylcyclopropyl | 3-Cl, 4-F |
| M.1291. | C(═S)NH₂ | 1-methylsulfonylcyclopropyl | 2-Cl, 4,5-F₂ |
| M.1292. | C(═S)NH₂ | 1-methylsulfonylcyclopropyl | 2-Cl, 4-Br |
| M.1293. | C(═S)NH₂ | 1-methylsulfonylcyclopropyl | 2-Cl, 5-Br |
| M.1294. | C(═S)NH₂ | 1-methylsulfonylcyclopropyl | 3-Cl, 4-Br |
| M.1295. | C(═S)NH₂ | 1-methylsulfonylcyclopropyl | 2-F, 4-Br |
| M.1296. | C(═S)NH₂ | 1-methylsulfonylcyclopropyl | 2-F, 5-Br |
| M.1297. | C(═S)NH₂ | 1-methylsulfonylcyclopropyl | 3-F, 4-Br |
| M.1298. | C(═S)NH₂ | 1-methylsulfonylcyclopropyl | 2-Br, 4-F |
| M.1299. | C(═S)NH₂ | 1-methylsulfonylcyclopropyl | 3-Br, 4-F |
| M.1300. | C(═S)NH₂ | 1-methylsulfonylcyclopropyl | 2-Br, 5-Cl |
| M.1301. | C(═S)NH₂ | 1-methylsulfonylcyclopropyl | 3-Br, 4-Cl |
| M.1302. | C(═S)NH₂ | 1-methylsulfonylcyclopropyl | 2-Br, 4,5-F₂ |
| M.1303. | C(═S)NH₂ | 1-methylsulfonylcyclopropyl | 3- ═N—O—N═ -4 |
| M.1304. | C(═S)NH₂ | 1-methylsulfonylcyclopropyl | 4-CHF₂ |
| M.1305. | C(═S)NH₂ | 1-methylsulfonylcyclopropyl | 2-F, 3-CF₃ |
| M.1306. | C(═S)NH₂ | 1-methylsulfonylcyclopropyl | 2-F, 4-CF₃ |
| M.1307. | C(═S)NH₂ | 1-methylsulfonylcyclopropyl | 2-F, 5-CF₃ |
| M.1308. | C(═S)NH₂ | 1-methylsulfonylcyclopropyl | 3-F, 4-CF₃ |
| M.1309. | C(═S)NH₂ | 1-methylsulfonylcyclopropyl | 3-F, 5-CF₃ |
| M.1310. | C(═S)NH₂ | 1-methylsulfonylcyclopropyl | 4-F, 3-CF₃ |
| M.1311. | C(═S)NH₂ | 1-methylsulfonylcyclopropyl | 3,4-F₂, 5-CF₃ |
| M.1312. | C(═S)NH₂ | 1-methylsulfonylcyclopropyl | 2-Cl, 3-CF₃ |
| M.1313. | C(═S)NH₂ | 1-methylsulfonylcyclopropyl | 2-Cl, 4-CF₃ |
| M.1314. | C(═S)NH₂ | 1-methylsulfonylcyclopropyl | 2-Cl, 5-CF₃ |
| M.1315. | C(═S)NH₂ | 1-methylsulfonylcyclopropyl | 3-Cl, 5-CF₃ |
| M.1316. | C(═S)NH₂ | 1-methylsulfonylcyclopropyl | 4-Cl, 3-CF₃ |

Table 1: This table discloses 1316 compounds 1.1 to 1.1316 of the formula (I-a), wherein Y is 5-methyl-1,2,4-oxadiazol-3-yl, X is hydrogen and R¹, R³ and R⁵ are as defined in Table M. For example, compound No. 1.1 has the following structure:

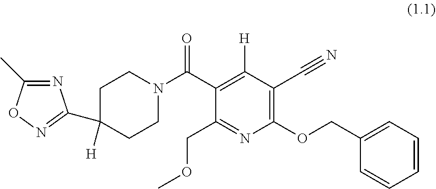 [SMILES: COCc1nc(OCc2ccccc2)c(C#N)cc1C(=O)N1CCC(c2noc(C)n2)CC1]

Table 2: This table discloses 1316 compounds 2.1 to 2.1316 of the formula (I-a), wherein Y is 1-methyl-1,2,4-triazole-3-yl, X is hydrogen and R¹, R³ and R⁵ are as defined in Table M.

Table 3: This table discloses 1316 compounds 3.1 to 3.1316 of the formula (I-a), wherein Y is 3,5-dichloropyridine-2-yl, X is hydrogen and R¹, R³ and R⁵ are as defined in Table M.

Table 4: This table discloses 1316 compounds 4.1 to 4.1316 of the formula (I-a), wherein Y is 3-chloro-5-(trifluoromethyl)pyridine-2-yl, X is hydrogen and R¹, R³ and R⁵ are as defined in Table M.

Table 5: This table discloses 1316 compounds 5.1 to 5.1316 of the formula (I-a), wherein Y is 4-chloro-phenyl-1-yl, X is hydrogen and R¹, R³ and R⁵ are as defined in Table M.

Table 6: This table discloses 1316 compounds 6.1 to 6.1316 of the formula (I-a), wherein Y is 4-chloro-phenyl-1-yl, X is hydroxyl and R¹, R³ and R⁵ are as defined in Table M.

Table 7: This table discloses 1316 compounds 7.1 to 7.1316 of the formula (I-a), wherein Y is methylsulfanylmethyl, X is hydrogen and R¹, R³ and R⁵ are as defined in Table M.

Table 8: This table discloses 1316 compounds 8.1 to 8.1316 of the formula (I-a), wherein Y is methylsulfinylmethyl, X is hydrogen and R¹, R³ and R⁵ are as defined in Table M.

Table 9: This table discloses 1316 compounds 9.1 to 9.1316 of the formula (I-a), wherein Y is methylsulfonylmethyl, X is hydrogen and R¹, R³ and R⁵ are as defined in Table M.

Table 10: This table discloses 1316 compounds 10.1 to 10.1316 of the formula (I-a), wherein Y is ethylsulfanylmethyl, X is hydrogen and R¹, R³ and R⁵ are as defined in Table M.

Table 11: This table discloses 1316 compounds 11.1 to 11.1316 of the formula (I-a), wherein Y is ethylsulfinylmethyl, X is hydrogen and R¹, R³ and R⁵ are as defined in Table M.

Table 12: This table discloses 1316 compounds 12.1 to 12.1316 of the formula (I-a), wherein Y is ethylsulfonylmethyl, X is hydrogen and R¹, R³ and R⁵ are as defined in Table M.

Table 13: This table discloses 1316 compounds 13.1 to 13.1316 of the formula (I-a), wherein Y is 1,1-dioxo-1,2-thiazolidin-2-yl, X is hydrogen and R¹, R³ and R⁵ are as defined in Table M.

Table 14: This table discloses 1316 compounds 14.1 to 14.1316 of the formula (I-a), wherein Y is dimethylcarbamoyl, X is hydrogen and R¹, R³ and R⁵ are as defined in Table M.

Table 15: This table discloses 1316 compounds 15.1 to 15.1316 of the formula (I-a), wherein Y is methanesulfonamido, X is hydrogen and R¹, R³ and R⁵ are as defined in Table M.

Table 16: This table discloses 1316 compounds 16.1 to 16.1316 of the formula (I-a), wherein Y is ethoxymethyl, X is hydrogen and R¹, R³ and R⁵ are as defined in Table M.

Table 17: This table discloses 1316 compounds 17.1 to 17.1316 of the formula (I-a), wherein Y is methoxyethyl, X is hydrogen and R¹, R³ and R⁵ are as defined in Table M.

Table 18: This table discloses 1316 compounds 18.1 to 18.1316 of the formula (I-a), wherein Y is 2-methyl-1,2,4-triazole-3-yl, X is hydrogen and R¹, R³ and R⁵ are as defined in Table M.

Table 19: This table discloses 1316 compounds 19.1 to 19.1316 of the formula (I-a), wherein Y is 1-methylpyrazol-4-yl, X is hydrogen and R¹, R³ and R⁵ are as defined in Table M.

Table 20: This table discloses 1316 compounds 20.1 to 20.1316 of the formula (I-a), wherein Y is 4-methylpyrazol-1-yl, X is hydrogen and R¹, R³ and R⁵ are as defined in Table M.

Table 21: This table discloses 1316 compounds 21.1 to 21.1316 of the formula (I-a), wherein Y is 3-methyl-1,2,4-oxadiazol-5-yl, X is hydrogen and R¹, R³ and R⁵ are as defined in Table M.

Table 22: This table discloses 1316 compounds 22.1 to 22.1316 of the formula (I-a), wherein Y is 1-methylpyrazol-3-yl, X is hydrogen and R¹, R³ and R⁵ are as defined in Table M.

Table 23: This table discloses 1316 compounds 23.1 to 23.1316 of the formula (I-a), wherein Y is 3-methylisoxazole-5-yl, X is hydrogen and R¹, R³ and R⁵ are as defined in Table M.

Table 24: This table discloses 1316 compounds 24.1 to 24.1316 of the formula (I-a), wherein Y is 2-oxooxazolidine-3-yl, X is hydrogen and R¹, R³ and R⁵ are as defined in Table M.

Table 25: This table discloses 1316 compounds 25.1 to 25.1316 of the formula (I-a), wherein Y is 2-oxopyrrolidine-1-yl, X is hydrogen and R¹, R³ and R⁵ are as defined in Table M.

Table 26: This table discloses 1316 compounds 26.1 to 26.1316 of the formula (I-a), wherein Y is 1,2,3-triazole-2-yl, X is hydrogen and R¹, R³ and R⁵ are as defined in Table M.

Table 27: This table discloses 1316 compounds 27.1 to 27.1316 of the formula (I-a), wherein Y is 1,2,4-triazole-3-yl, X is hydrogen and R¹, R³ and R⁵ are as defined in Table M.

Table 28: This table discloses 1316 compounds 28.1 to 28.1316 of the formula (I-a), wherein Y is 2-oxo-5,5-dimethyl-oxazolidine-3-yl, X is hydrogen and R¹, R³ and R⁵ are as defined in Table M.

Table 29: This table discloses 1316 compounds 29.1 to 29.1316 of the formula (I-a), wherein Y is methylamino-carbonyl, X is hydrogen and R¹, R³ and R⁵ are as defined in Table M.

Table 30: This table discloses 1316 compounds 30.1 to 30.1316 of the formula (I-a), wherein Y is 2-oxopiperidine-1-yl, X is hydrogen and R¹, R³ and R⁵ are as defined in Table M.

Table 31: This table discloses 1316 compounds 31.1 to 31.1316 of the formula (I-a), wherein Y is acetamido, X is hydrogen and R¹, R³ and R⁵ are as defined in Table M.

Table 32: This table discloses 1316 compounds 32.1 to 32.1316 of the formula (I-a), wherein Y is 2-methylpropanoylamino, X is hydrogen and R¹, R³ and R⁵ are as defined in Table M.

Table 33: This table discloses 1316 compounds 33.1 to 33.1316 of the formula (I-a), wherein Y is 1,2,3-triazole-1-yl, X is hydrogen and R¹, R³ and R⁵ are as defined in Table M.

Table 34: This table discloses 1316 compounds 34.1 to 34.1316 of the formula (I-a), wherein Y is 5-methylisoxazole-3-yl, X is hydrogen and R¹, R³ and R⁵ are as defined in Table M.

Table 35: This table discloses 1316 compounds 35.1 to 35.1316 of the formula (I-a), wherein Y is 2-oxoazetidine-1-yl, X is hydrogen and R¹, R³ and R⁵ are as defined in Table M.

Table 36: This table discloses 1316 compounds 36.1 to 36.1316 of the formula (I-a), wherein Y is 3-methylpyrazol-1-yl, X is hydrogen and R¹, R³ and R⁵ are as defined in Table M.

Table 37: This table discloses 1316 compounds 37.1 to 37.1316 of the formula (I-a), wherein Y is 1-methyl-5-oxo-4H-pyrazol-3-yl, X is hydrogen and R¹, R³ and R⁵ are as defined in Table M.

Table 38: This table discloses 1316 compounds 38.1 to 38.1316 of the formula (I-a), wherein Y is 3-chloro-1,2,4-triazole-1-yl, X is hydrogen and R¹, R³ and R⁵ are as defined in Table M.

Table 39: This table discloses 1316 compounds 39.1 to 39.1316 of the formula (I-a), wherein Y is 3-methyl-1,2,4-triazole-1-yl, X is hydrogen and R¹, R³ and R⁵ are as defined in Table M.

Table 40: This table discloses 1316 compounds 40.1 to 40.1316 of the formula (I-a), wherein Y is 3-methyl-5-oxo-4H-pyrazol-1-yl, X is hydrogen and R¹, R³ and R⁵ are as defined in Table M.

Table 41: This table discloses 1316 compounds 41.1 to 41.1316 of the formula (I-a), wherein Y is 3-methyl-2-oxo-imidazolidine-1-yl, X is hydrogen and R¹, R³ and R⁵ are as defined in Table M.

Table 42: This table discloses 1316 compounds 42.1 to 42.1316 of the formula (I-a), wherein Y is 5-methyl-1,1-dioxo-1,2,5-thiadiazolidine-2-yl, X is hydrogen and R¹, R³ and R⁵ are as defined in Table M.

Table 43: This table discloses 1316 compounds 43.1 to 43.1316 of the formula (I-a), wherein Y is 5-ethyl-1,1-dioxo-1,2,5-thiadiazolidine-2-yl, X is hydrogen and R¹, R³ and R⁵ are as defined in Table M.

Table 44: This table discloses 1316 compounds 44.1 to 44.1316 of the formula (I-a), wherein Y is 1,2-dimethylimidazole-4-yl, X is hydrogen and R¹, R³ and R⁵ are as defined in Table M.

Table 45: This table discloses 1316 compounds 45.1 to 45.1316 of the formula (I-a), wherein Y is 1-methylimidazole-4-yl, X is hydrogen and R¹, R³ and R⁵ are as defined in Table M.

Table 46: This table discloses 1316 compounds 46.1 to 46.1316 of the formula (I-a), wherein Y is 4-methyltriazole-1-yl, X is hydrogen and R¹, R³ and R⁵ are as defined in Table M.

Table 47: This table discloses 1316 compounds 47.1 to 47.1316 of the formula (I-a), wherein Y is 2-methyl-3-oxo-pyrazolidine-1-yl, X is hydrogen and R¹, R³ and R⁵ are as defined in Table M.

Table 48: This table discloses 1316 compounds 48.1 to 48.1316 of the formula (I-a), wherein Y is 1,2,4-triazole-4-yl, X is hydrogen and R¹, R³ and R⁵ are as defined in Table M.

Specific examples of compounds of the present invention are represented by the formula (I-b) in the following Tables 49 to 81:

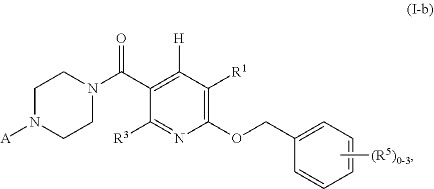

wherein R¹, R³ and R⁵ are as defined above in table M.

Each of Tables 49 to 81 below, consists of 1316 compounds of the formula (I-b) in which R¹, R³ and R⁵ have the values given in each row in Table M, and A has the values given in the relevant Tables 49 to 81. Thus compound 49.1 corresponds to a compound of formula (I-b) where R¹, R³ and R⁵ are as defined in row 1 of Table M and where A is as defined in Table 49; compound 60.14 corresponds to a compound of formula (I-b) where R¹, R³ and R⁵ are as defined in row 14 of Table M and where A is as defined in Table 60; and so on.

Table 49: This table discloses 1316 compounds 49.1 to 49.1316 of the formula (I-b), wherein A is 3,5-dichloropyridine-2-yl, and R¹, R³ and R⁵ are as defined in Table M. For example, compound No. 55.1 has the following structure:

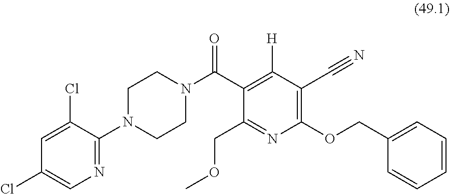 [SMILES: COCc1nc(OCc2ccccc2)c(C#N)cc1C(=O)N1CCN(c2ncc(Cl)cc2Cl)CC1]

Table 50: This table discloses 1316 compounds 50.1 to 50.1316 of the formula (I-b), wherein A is 3-chloro-5-(trifluoromethyl)pyridine-2-yl, and R¹, R³ and R⁵ are as defined in Table M.

Table 51: This table discloses 1316 compounds 51.1 to 51.1316 of the formula (I-b), wherein A is 3-chloro-pyridine-2-yl, and R¹, R³ and R⁵ are as defined in Table M.

Table 52: This table discloses 1316 compounds 52.1 to 52.1316 of the formula (I-b), wherein A is 5-fluoro-pyridine-2-yl, and R¹, R³ and R⁵ are as defined in Table M.

Table 53: This table discloses 1316 compounds 53.1 to 53.1316 of the formula (I-b), wherein A is cyanoethyl, and R¹, R³ and R⁵ are as defined in Table M.

Table 54: This table discloses 1316 compounds 54.1 to 54.1316 of the formula (I-b), wherein A is cyanopropyl, and R¹, R³ and R⁵ are as defined in Table M.

Table 55: This table discloses 1316 compounds 55.1 to 55.1316 of the formula (I-b), wherein A is vinyloxycarbonyl, and R¹, R³ and R⁵ are as defined in Table M.

Table 56: This table discloses 1316 compounds 56.1 to 56.1316 of the formula (I-b), wherein A is tert.butyloxycarbonyl and R¹, R³ and R⁵ are as defined in Table M.

Table 57: This table discloses 1316 compounds 57.1 to 57.1316 of the formula (I-b), wherein A is 4-fluoro-phenyl, and R¹, R³ and R⁵ are as defined in Table M.

Table 58: This table discloses 1316 compounds 58.1 to 58.1316 of the formula (I-b), wherein A is 4-chloro-phenyl, and R¹, R³ and R⁵ are as defined in Table M.

Table 59: This table discloses 1316 compounds 59.1 to 59.1316 of the formula (I-b), wherein A is ethylsulfanylmethyl, and R¹, R³ and R⁵ are as defined in Table M.

Table 60: This table discloses 1316 compounds 60.1 to 60.1316 of the formula (I-b), wherein A is ethylsulfinylmethyl, and R¹, R³ and R⁵ are as defined in Table M.

Table 61: This table discloses 1316 compounds 61.1 to 61.1316 of the formula (I-b), wherein A is ethylsulfonylmethyl, and R¹, R³ and R⁵ are as defined in Table M.

Table 62: This table discloses 1316 compounds 62.1 to 62.1316 of the formula (I-b), wherein A is 5-methyl-1,2,4-oxadiazol-3-yl, and R¹, R³ and R⁵ are as defined in Table M.

Table 63: This table discloses 1316 compounds 63.1 to 63.1316 of the formula (I-b), wherein A is 1-methylpyrazol-4-yl, and R¹, R³ and R⁵ are as defined in Table M.

Table 64: This table discloses 1316 compounds 64.1 to 64.1316 of the formula (I-b), wherein A is 3-methyl-1,2,4-oxadiazol-5-yl, and R¹, R³ and R⁵ are as defined in Table M.

Table 65: This table discloses 1316 compounds 65.1 to 74.1316 of the formula (I-b), wherein A is methylsulfamoyl, and R¹, R³ and R⁵ are as defined in Table M.

Table 66: This table discloses 1316 compounds 66.1 to 66.1316 of the formula (I-b), wherein A is ethylsulfamoyl, and R¹, R³ and R⁵ are as defined in Table M.

Table 67: This table discloses 1316 compounds 67.1 to 67.1316 of the formula (I-b), wherein A is dimethylsulfamoyl, and R¹, R³ and R⁵ are as defined in Table M.

Table 68: This table discloses 1316 compounds 68.1 to 68.1316 of the formula (I-b), wherein A is ethyl(methyl)sulfamoyl, and R¹, R³ and R⁵ are as defined in Table M.

Table 69: This table discloses 1316 compounds 69.1 to 69.1316 of the formula (I-b), wherein A is 1-methylpyrazol-3-yl, and R¹, R³ and R⁵ are as defined in Table M.

Table 70: This table discloses 1316 compounds 70.1 to 70.1316 of the formula (I-b), wherein A is methylsulfonyl, and R¹, R³ and R⁵ are as defined in Table M.

Table 71: This table discloses 1316 compounds 71.1 to 71.1316 of the formula (I-b), wherein A is ethylsulfonyl, and R¹, R³ and R⁵ are as defined in Table M.

Table 72: This table discloses 1316 compounds 72.1 to 72.1316 of the formula (I-b), wherein A is propylsulfonyl, and R¹, R³ and R⁵ are as defined in Table M.

Table 73: This table discloses 1316 compounds 73.1 to 73.1316 of the formula (I-b), wherein A is iso-propylsulfonyl, and R¹, R³ and R⁵ are as defined in Table M.

Table 74: This table discloses 1316 compounds 74.1 to 74.1316 of the formula (I-b), wherein A is cyclopropylsulfonyl, and R¹, R³ and R⁵ are as defined in Table M.

Table 75: This table discloses 1316 compounds 75.1 to 75.1316 of the formula (I-b), wherein A is 1,2-dimethylimidazole-4-yl, and R¹, R³ and R⁵ are as defined in Table M.

Table 76: This table discloses 1316 compounds 76.1 to 76.1316 of the formula (I-b), wherein A is 1-methylimidazole-4-yl, and R¹, R³ and R⁵ are as defined in Table M.

Table 77: This table discloses 1316 compounds 77.1 to 77.1316 of the formula (I-b), wherein A is 1-cyanocyclopropyl, and R¹, R³ and R⁵ are as defined in Table M.

Table 78: This table discloses 1316 compounds 78.1 to 78.1316 of the formula (I-b), wherein A is 1-cyano-1-methyl-ethyl, and R¹, R³ and R⁵ are as defined in Table M.

Table 79: This table discloses 1316 compounds 79.1 to 79.1316 of the formula (I-b), wherein A is (1-cyanocyclopropyl)methyl, and R¹, R³ and R⁵ are as defined in Table M.

Table 80: This table discloses 1316 compounds 80.1 to 80.1316 of the formula (I-b), wherein A is methylsulfanylmethyl, and R¹, R³ and R⁵ are as defined in Table M.

Table 81: This table discloses 1316 compounds 81.1 to 81.1316 of the formula (I-b), wherein A is methylsulfonylmethyl, and R¹, R³ and R⁵ are as defined in Table M.

Also made available are certain intermediate compounds of the amine of formulae III-a, V-a, VI-a, VI-b, VII-a, IX-a, XI-a, XI-b, XII-a, XII-b, XIII-a, XIV-a, some of which are novel.

Accordingly, made available herein are:
compounds of formula III-a

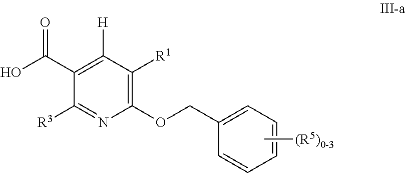

wherein R¹, R³ and R⁵ are defined in each row of Table M;
compounds of formula V-a

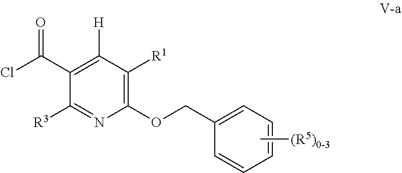

wherein R¹, R³ and R⁵ are defined in each row of Table M;
compounds of formula VI-a

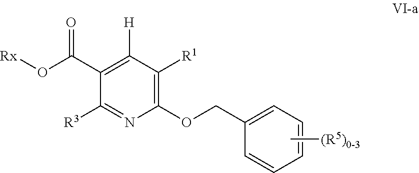

wherein R¹, R³, and R⁵ are defined in each row of Table M, and R^{x} is selected from methyl, ethyl, propyl, iso-propyl, butyl, iso-butyl and tert-butyl;
compounds of formula VII-a

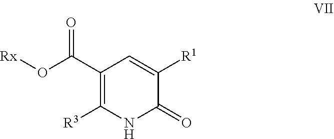

wherein R¹ and R³ are defined in each row of Table M, and R^{x} is selected from methyl, ethyl, propyl, iso-propyl, butyl, iso-butyl and tert-butyl;
compounds of formula IX-a

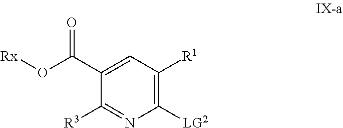

wherein R¹ and R³ are defined in each row of Table M, R^{x} is selected from methyl, ethyl, propyl, iso-propyl, butyl, iso-butyl and tert-butyl, and LG² is chloro;
compounds of formula XI-a

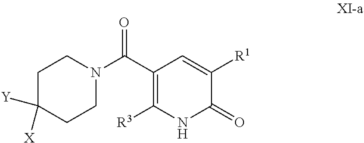

wherein R¹, R³, X and Y are defined in any one of Tables 1 to 48;
compounds of formula XI-b

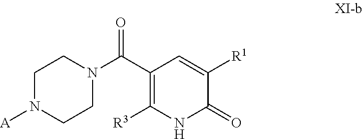

wherein R¹, R³ and A are defined in any one of Tables 49 to 81;
compounds of formula XII-a

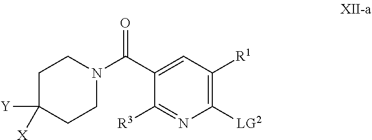

wherein R¹, R³, X and Y are defined in any one of Tables 1 to 48, and LG² is chloro, bromo or fluoro;
compounds of formula XII-b

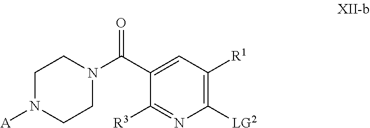

wherein R¹, R³ and A are defined in any one of Tables 49 to 81, and LG² is chloro, bromo or fluoro;
compounds of formula XIII-a

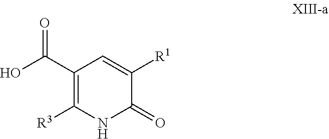

wherein R¹ and R³ are defined in each row of Table M; and
compounds of formula XIV-a

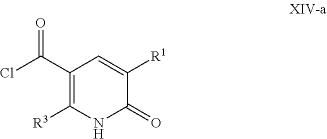

wherein R¹ and R³ are defined in each row of Table M.

The compounds of formula (I) according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the above mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp orjute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

In a particular embodiment, a compound of the formula (I) controls mites, rust mites and spider mites in crops, tress, and plants selected from vegetables (especially tomatoes and cucurbits), citrus, pome fruits, stone fruit, tree nuts, cotton, tropical crops, avocados, ornamentals, beans, soybean, strawberry, and grapes.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. porrum, A. ascalonicum, A. fistulosum), Anthriscus cerefolium, Apium graveolus, Asparagus officinalis, Beta vulgarus, Brassica spp. (B. oleracea, B. pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

The compounds of formula (I) are particularly suitable for control of mites, spider mites and rust mites, for example, Acarapis spp; Acarapis woodi; Acarus siro; Acarus spp; Aceria sheldoni; Aculops pelekassi; Aculops spp; Aculus schlechtendali; Aculus spp; Amblyseius fallacis; Brevipalpus spp; Brevipalpus phoenicis; Bryobia praetiosa; Bryobia rubrioculus; Caloglyphus spp; Cheyletiella blakei; Cheyletiella spp; Cheyletiella yasguri; Chorioptes bovis; Chorioptes spp; Cytodites spp; Demodex bovis; Demodex caballi; Demodex canis; Demodex caprae; Demodex equi; Demodex ovis; Demodex spp; Demodex suis; Dermanyssus gallinae; Dermanyssus spp; Eotetranychus spp; Eotetranychus willamettei; Epitrimerus pyri; Eriophyes ribis; Eriophyes spp; Eriophyes vitis; Eutetranychus spp; Halotydeus destructor; Hemitarsonemus spp; Knemidocoptes spp; Laminosioptes spp; Listrophorus spp; Myobia spp; Neoschongastia xerothermobia; Neotrombicula autumnalis; Neotrombicula desaleri; Notoedres cati; Notoedres spp; Oligonychus coffeae; Oligonychus ilicis; Oligonychus spp; Ornithocheyletia spp; Ornithonyssus bursa; Ornithonyssus spp; Ornithonyssus sylviarum; Otodectes cynotis; Otodectes spp; Panonychus citri; Panonychus spp; Panonychus ulmi; Phyllocoptruta oleivora; Phyllocoptruta spp.; Phytoseiulus spp.; Pneumonyssoides caninum; Polyphagotarsonemus latus; Polyphagotarsonemus spp; Psorergates ovis; Psorergates spp; Psoroptes cuniculi; Psoroptes equi; Psoroptes ovis; Psoroptes spp; Pterolichus spp; Raillietia spp; Rhizoglyphus spp; Sarcoptes bovis; Sarcoptes canis; Sarcoptes caprae; Sarcoptes equi; Sarcoptes ovis; Sarcoptes rupicaprae; Sarcoptes spp; Sarcoptes suis; Steneotarsonemus spinki; Steneotarsonemus spp; Sternostoma spp; Tarsonemus spp; Tetranychus cinnabarinus; Tetranychus kanzawai; Tetranychus spp; Tetranychus urticae; Trombicula akamushi; Trombicula spp; Typhlodromus occidentalis; Tyrophagus spp; Varroa jacobsoni; Varroa spp; Vasates lycopersici; and Zetzellia mali.

In an embodiment, a compound of formula (I) are especially suitable for controlling one or more of: Aceria sheldoni; Aculus lycopersici; Aculus pelekassi; Aculus schlechtendali; Brevipalpus phoenicis; Brevipalpus spp.; Bryobia rubrioculus; Eotetranychus carpini; Eotetranychus spp.; Epitrimerus pyri; Eriophyes piri; Eriophyes spp.; Eriophyes vitis; Eutetranychus africanus; Eutetranychus orientalis; Oligonychus pratensis; Panonychus citri; Panonychus ulmi; Phyllocoptes vitis; Phyllocoptruta oleivora; Polyphagotarsonemus latus; Tetranychus cinnabarinus; Tetranychus kanzawai; Tetranychus spp.; and Tetranychus urticae.

In a further embodiment, a compound of formula (I) are more especially suitable for controlling one or more of: Aceria sheldoni; Aculus pelekassi; Brevipalpus phoenicis; Brevipalpus spp.; Eriophyes piri; Eriophyes vitis; Eutetranychus africanus; Eutetranychus orientalis; Oligonychus pratensis; Panonychus ulmi; Phyllocoptes vitis; Phyllocoptruta oleivora; Polyphagotarsonemus latus; Tetranychus cinnabarinus; Tetranychus kanzawai; Tetranychus spp.; and Tetranychus urticae.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603 x MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention provides a compound of the first aspect for use in therapy. The present invention provides a compound of the first aspect, for use in controlling parasites in or on an animal.

The present invention further provides a compound of the first aspect, for use in controlling ectoparasites on an animal. The present invention further provides a compound of the first aspect, for use in preventing and/or treating diseases transmitted by ectoparasites.

The present invention provides the use of a compound of the first aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.

The present invention provides the use of a compound of the first aspect, in controlling parasites in or on an animal. The present invention further provides the use of a compound of the first aspect, in controlling ectoparasites on an animal.

The term “controlling” when used in context of parasites in or on an animal refers to reducing the number of pests or parasites, eliminating pests or parasites and/or preventing further pest or parasite infestation.

The term “treating” when used in context of parasites in or on an animal refers to restraining, slowing, stopping or reversing the progression or severity of an existing symptom or disease.

The term “preventing” when used in context of parasites in or on an animal refers to the avoidance of a symptom or disease developing in the animal.

The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camellids, pigs, sheep, goats and horses. Companion animals include, but are not limited to, dogs, cats and rabbits.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example Haematobia irritans; Stomoxys; Lucilia; midges; and mosquitoes. Members of the Phthiraptera class include, but are not limited to, blood sucking lice and chewing lice, for example Bovicola ovis and Bovicola bovis.

The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of or involvement or the severity of the disease or disorder; the response of the individual; the particular compound administered; the mode of administration; the bioavailability characteristics of the preparation administered; the dose regimen selected; the use of concomitant medication; and other relevant circumstances.

The compounds of the invention may be administered to the animal by any route which has the desired effect including, but not limited to topically, orally, parenterally and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.

Salt forms of the compounds of the invention include both pharmaceutically acceptable salts and veterinary acceptable salts, which can be different to agrochemically acceptable salts.

Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina. The compounds of formulae I, and I′a, or salts thereof, are especially suitable for controlling one or more pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae. In a preferred embodiment of each aspect, a compound TX (where the abbreviation “TX” means “one compound selected from the compounds defined in Tables 1 to 81 and Tables P1 to P6”) controls one or more of pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae.

The compounds of formulae I, and I′a, or salts thereof, are especially suitable for controlling one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp. In a preferred embodiment of each aspect, a compound TX (where the abbreviation “TX” means “one compound selected from the compounds defined in Tables 1 to 81 and Tables P1 to P6”) controls one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp.

The compounds of formulae I, and I′a, or salts thereof, are especially suitable for controlling one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padi, and Chilo suppressalis.

In a preferred embodiment of each aspect, a compound TX (where the abbreviation “TX” means “one compound selected from the compounds defined in Tables 1 to 81 and Tables P1 to P6”) controls one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padia, and Chilo Suppressalis, such as Spodoptera littoralis+TX, Plutella xylostella+TX; Frankliniella occidentalis+TX, Thrips tabaci+TX, Euschistus heros+TX, Cydia pomonella+TX, Nilaparvata lugens+TX, Myzus persicae+TX, Chrysodeixis includens+TX, Aphis craccivora+TX, Diabrotica balteata+TX, Rhopalosiphum Padi+TX, and Chilo suppressalis+TX.

In an embodiment, of each aspect, one compound from Tables 1 to 81 and Tables P1 to P6 is suitable for controlling Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padia, and Chilo Suppressalis in cotton, vegetable, maize, cereal, rice and soya crops.

In an embodiment, one compound from Tables 1 to 81 and Tables P1 to P6 is suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile (against non-target organisms above and below ground (such as fish, birds and bees), improved physico-chemical properties, or increased biodegradability). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively).

Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

Emulsifiable concentrates:
active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene | 3% |
| oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

### LCMS Methods:

### Method 1:

Spectra were recorded on a Mass Spectrometer from Waters (SQD Single quadrupole mass spectrometer) equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: Quaternary pump, heated column compartment and diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0-0.2 min 10-50% B; 0.2-0.7 min 50-100% B; 0.7-1.3 min 100% B; 1.3-1.4 min 100-10% B; 1.4-1.6 min 10% B; Flow (mL/min) 0.6.

### Method 2:

Spectra were recorded on a Mass Spectrometer from Agilent Technologies (6410 Triple Quadrupole mass spectrometer) equipped with an equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: quaternary pump, heated column compartment and diode-array detector. Column: KINETEX EVO C18, 2.6 μm, 50×4.6 mm, Temp: 40° C., DAD Wavelength range (nm): 210 to 400, Solvent Gradient: A=95% (water+0.1% HCOOH): 5% Acetonitrile, B=Acetonitrile with 0.1% HCOOH: gradient: 0 min 10% B; 0.9-1.8 min 100% B; 1.8-2.2 min 100-10% B; 2.2-2.5 min 10% B, Flow rate (mL/min) 1.8.

### Method 3:

Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Extractor: V (No extractor voltage for QDa detector) Source Temperature: 120° C., Desolvation Temperature: 600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Waters UPLC HSS T3, 1.8 μm, 30×2.1 mm, Temp: 40° C., PDA Wavelength range (nm): 230 to 400, Solvent Gradient: A=Water with 0.1% formic acid: Acetonitrile: 95:5 v/v, B=Acetonitrile with 0.05% formic acid: Gradient: 0 min-1.0 min, 10% B-90% A; 1.0 min-4.50 min 10%-100% B; 4.51 min-5.30 min, 100% B, 0% A; 5.31 min-5.50 min 100%-10% B; 5.51 min-6.00 min 0.10% B, 90% A; Flow (ml/min) 0.6.

### Method 4:

Spectra were recorded on a Mass Spectrometer from Agilent Technologies (6410 Triple Quadrupole mass spectrometer) equipped with an electrospray source (Polarity: positive or negative ions, MS2 Scan, Capillary: 7.00 kV, Fragmentor: 120 V, Desolvatation Temperature: 350° C., Gas Flow: 11 L/min, Nebulizer Gas: 40 psi, Mass range: 110 to 1000 Da) and a 1200 Series HPLC from Agilent: quaternary pump, heated column compartment and diode-array detector. Column: KINETEX EVO C18, 2.6 μm, 50×4.6 mm, Temp: 40° C., Detector VWD Wavelength: 254 nm, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

### Method 5:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Method 6:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 □m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

The following abbreviations are used in the experimental description below: s=singlet, d=doublet, t=triplet, q=quartet, m=multiplet, RT=retention time, min=minutes.

### Example P1: 2-[[2-fluoro-5-(trifluoromethyl)phenyl]methoxy]-6-(methoxymethyl)-5-[4-(5-methyl-1,2,4-oxadiazol-3-yl)piperidine-1-carbonyl]pyridine-3-carbonitrile (compound P1.2)

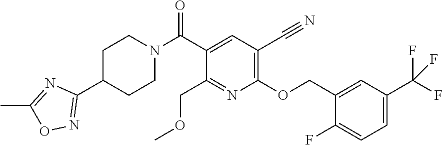 [SMILES: COCc1nc(OCc2cc(C(F)(F)F)ccc2F)c(C#N)cc1C(=O)N1CCC(c2noc(C)n2)CC1]

### Step 1: Methyl 5-cyano-6-hydroxy-2-(methoxymethyl) pyridine-3-carboxylate

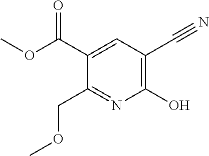 [SMILES: COCc1nc(O)c(C#N)cc1C(=O)OC]

A mixture of methyl 4-methoxy-3-oxo-butanoate (5.00 g, 34.2 mmol) and 1,1-dimethoxy-N,N-dimethyl-methanamine (56.45 mmol, 1.65 eq) was stirred overnight at room temperature. The reaction mixture was concentrated under reduced pressure. Sodium hydride in paraffin oil (2.1 g, 53 mmol, 60 mass %) was added to a mixture of 2-cyanoacetamide (4.2 g, 50 mmol) in tetrahydrofuran (79 mL). Then the residue obtained above was added to the mixture, and the mixture was stirred at room temperature for 18 hours. The reaction mixture was concentrated under reduced pressure, and to the obtained residue water was added. Then the mixture is acidified with hydrochloric acid. The deposited solid was collected and washed with water and hexane to obtain 3.6 g of methyl 5-cyano-6-hydroxy-2-(methoxymethyl)pyridine-3-carboxylate as a solid. ¹H-NMR [ppm] in DMSO-d₆: 3.37 (s, 3H), 3.79 (s, 3H), 4.77 (s, 2H), 8.47 (s, 1H), 12.43 (broad s, 1H).

### Step 2: Methyl 5-cyano-6-[[2-fluoro-5-(trifluoromethyl)phenyl]methoxy]-2-(methoxymethyl)pyridine-3-carboxylate

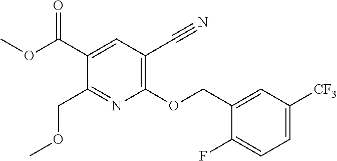 [SMILES: COCc1nc(OCc2cc(C(F)(F)F)ccc2F)c(C#N)cc1C(=O)OC]

To a solution of methyl 5-cyano-6-hydroxy-2-(methoxymethyl)pyridine-3-carboxylate (1.00 g, 4.5 mmol) in DMF (11.3 mL) was added 2-fluoro-5-(trifluoromethyl)-benzyl bromide (197 mg, 6.75 mmol, 1.5 eq) followed by addition of potassium carbonate (1.27 g, 9.0 mmol 2 eq). The reaction mixture was stirred at 60° C. for 3 hours and cooled to room temperature. The reaction mixture was diluted with water and extracted three times with ethyl acetate. The combined organic extracts were extracted with brine, dried over sodium sulphate and concentrated under vacuum After removal of the solvent the residue was purified by combi-flash silica-gel (30-40% ethyl acetate/cyclohexane) to afford 900 mg of methyl 5-cyano-6-[[2-fluoro-5-(trifluoromethyl)phenyl]methoxy]-2-(methoxymethyl)pyridine-3-carboxylate. ¹H-NMR [ppm] in CDCl₃: 3.52 (s, 3H), 3.95 (s, 3H), 4.94 (s, 2H), 5.73 (s, 2H), 7.19-7.32 (m, 1H), 7.55-7.75 (m, 1H), 7.99 (dd, 1H), 8.50 (s, 1H).

### Step 3: 5-Cyano-6-[[2-fluoro-5-(trifluoromethyl) phenyl]methoxy]-2-(methoxymethyl)pyridine-3-carboxylic acid

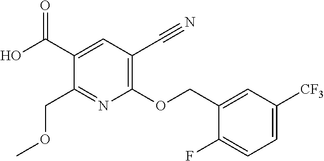 [SMILES: COCc1nc(OCc2cc(C(F)(F)F)ccc2F)c(C#N)cc1C(=O)O]

To a solution of methyl 5-cyano-6-[[2-fluoro-5-(trifluoromethyl)phenyl]methoxy]-2-(methoxymethyl)pyridine-3-carboxylate (900 mg, 2.26 mmol) in THE (9 mL)/water (4.5 mL) was added Lithium hydroxide (395 mg, 9.04 mmol, 4 eq). The reaction mixture was stirred at room temperature until complete consumption of the starting material (monitored by TLC). After removal of the solvent under reduced pressure the residue was dissolved in water and extracted with ethyl acetate (organic layer was discarded). The aqueous layer was acidified with hydrochloric acid and then extracted three times with ethyl acetate. The combined extracts were dried over sodium sulphate and concentrated to give 750 mg of 5-cyano-6-[[2-fluoro-5-(trifluoromethyl)phenyl]methoxy]-2-(methoxymethyl)pyridine-3-carboxylic acid as a white solid. ¹H-NMR [ppm] in DMSO-d₆: 3.36 (s, 3H), 4.83 (s, 2H), 5.69 (s, 2H), 7.54 (t, 1H), 7.83-7.90 (m, 1H), 8.13 (dd, 2.08 Hz, 1H), 8.62 (s, 1H), 13.5 (br, s, 1H).

### Step 4: 2-[[2-fluoro-5-(trifluoromethyl)phenyl]methoxy]-6-(methoxymethyl)-5-[4-(5-methyl-1,2,4-oxadiazol-3-yl)piperidine-1-carbonyl]pyridine-3-carbonitrile (compound P1.2)

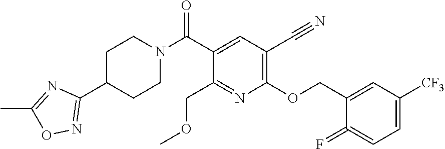

To a solution of 5-cyano-6-[[2-fluoro-5-(trifluoromethyl)phenyl]methoxy]-2-(methoxymethyl)pyridine-3-carboxylic acid (189 mg, 0.49 mmol) in acetonitrile (2.5 mL) was added the hydrochloride salt of 5-methyl-3-(4-piperidyl)-1,2,4-oxadiazole (100 mg, 0.49 mmol) which was synthesized as described in Nippon Soda patent application WO 2017195703. Subsequently COMU coupling reagent (243 mg, 0.54 mmol, 1.1 eq) and diisopropyl-ethyl-amine (0.27 mL, 1.47 mmol, 3 eq) were added. The reaction mixture was stirred at room temperature for 20 hours and then poured into an aqueous NaHCO₃ solution. The mixture was extracted 3 times with ethylacetate. The water layer was acidified with 2 N HCl and extracted 3 times with ethylacetate. The combined organic layers were washed with brine, dried over Na₂SO₄, filtered and concentrated under reduced pressure. The residue was purified via chromatography. 100 mg of 2-[[2-fluoro-5-(trifluoromethyl)phenyl]methoxy]-6-(methoxymethyl)-5-[4-(5-methyl-1,2,4-oxadiazol-3-yl)piperidine-1-carbonyl]pyridine-3-carbonitrile were obtained as a gum. ¹H-NMR [ppm] in CDCl₃: 1.86 (br d, 3H), 2.07-2.22 (m, 1H), 2.57 (s, 3H), 3.08 (br d, 2H), 3.16 (br s, 1H), 3.44 (s, 3H), 3.54 (br s, 1H), 4.47 (br s, 1H), 4.54 (br s, 1H), 4.68 (br s, 1H), 5.60 (s, 2H), 7.22 (t, 1H), 7.60-7.65 (m, 1H), 7.75 (s, 1H), 7.87 (d, 1H). LC-MS (method 2): RT=1.47 min; [M+H]⁺=534.1.

The compounds in Tables P1 were prepared as described in the examples above or similar methodology.

### Table P1: Compounds of Formula (I-c)

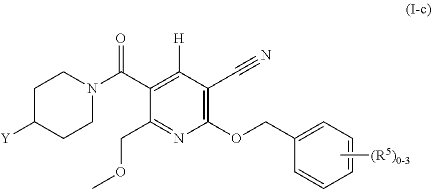

The compounds in Table P1 can be prepared as described in the examples above or similar methodology. The following abbreviations are used in the table below: RT=retention time, min=minutes.

TABLE P1

| Comp. | | | | LC-MS | |
|---|---|---|---|---|---|
| No | Y | R⁵ | [M + H]⁺ | RT (min) | Method |
| P1.1 | 1-methyl-1,2,4-triazole-3-yl | 2-F-5-CF₃ | 533.1 | 1.39 | 2 |
| P1.2 | 5-methyl-1,2,4-oxadiazol-3-yl | 2-F-5-CF₃ | 534.1 | 1.47 | 2 |
| P1.3 | 5-methyl-1,1-dioxo-1,2,5- | 3-O—CF₂—O-4 | 533.1 | 1.39 | 2 |
| | thiadiazolidin-2-yl) | | | | |
| P1.4 | 5-ethyl-1,1-dioxo-1,2,5- | 3-O—CF₂—O-4 | 534.1 | 1.47 | 2 |
| | thiadiazolidin-2-yl) | | | | |
| P1.5 | methoxyethyl | 3-O—CF₂—O-4 | 580 | 1.1 | 1 |
| P1.6 | 1-methylpyrazol-3-yl | 3-O—CF₂—O-4 | 594 | 1.07 | 1 |
| P1.7 | 5-methyl-1,2,4-oxadiazol-3-yl | 3-O—CF₂—O-4 | 504 | 1.54 | 4 |
| P1.8 | 5-methyl-1,2,4-oxadiazol-3-yl | 2-Cl, 4-O—CF₂—O-5 | 526 | 1.17 | 3 |
| P1.9 | 5-methyl-1,1-dioxo-1,2,5- | 3-F, 4-Br | 528 | 1.5 | 4 |
| | thiadiazolidin-2-yl) | | | | |
| P1.10 | 5-methyl-1,1-dioxo-1,2,5- | 2,4,5-F₃ | 562 | 1.18 | 3 |
| | thiadiazolidin-2-yl) | | | | |
| P1.11 | 5-methyl-1,1-dioxo-1,2,5- | 3,4-F₂ | 596 | 1.25 | 3 |
| | thiadiazolidin-2-yl) | | | | |
| P1.12 | 5-methyl-1,1-dioxo-1,2,5- | 3,4-Cl₂ | 554 | 1.05 | 1 |
| | thiadiazolidin-2-yl) | | | | |
| P1.13 | 5-methyl-1,1-dioxo-1,2,5- | 2,5-Cl₂ | 536 | 1.13 | 3 |
| | thiadiazolidin-2-yl) | | | | |
| P1.14 | 1-methylpyrazol-3-yl | 2-Cl, 4-O—CF₂—O-5 | 568 | 1.18 | 3 |
| P1.15 | 5-methyl-1,1-dioxo-1,2,5- | 2-Cl, 4-O—CF₂—O-5 | 568 | 1.19 | 3 |
| | thiadiazolidin-2-yl) | | | | |
| P1.16 | methoxyethyl | 3,4-F₂ | 560 | 1.19 | 1 |
| P1.17 | methoxyethyl | 2,5-Cl₂ | 614 | 1.18 | 3 |
| P1.18 | methoxyethyl | 2,4,5-F₃ | 460 | 1.16 | 1 |
| P1.19 | methoxyethyl | 3,4-Cl₂ | 492 | 1.27 | 1 |
| P1.20 | methoxyethyl | 2-Cl, 4-O—CF₂—O-5 | 478 | 1.23 | 1 |
| P1.21 | 5-methyl-1,2,4-oxadiazol-3-yl | 4-cyclopropyl | 492 | 1.23 | 1 |
| P1.22 | 5-methyl-1,2,4-oxadiazol-3-yl | 2-F, 5-Cl | 538 | 1.22 | 1 |
| P1.23 | 5-methyl-1,2,4-oxadiazol-3-yl | 3-Br, 4-F | 488.4 | 1.73 | 5 |
| P1.24 | 5-methyl-1,2,4-oxadiazol-3-yl | 3-F, 4-Br | 500.32 | 1.64 | 5 |
| P1.25 | 5-methyl-1,2,4-oxadiazol-3-yl | 3,4-Cl₂ | 544.3 | 1.67 | 5 |
| P1.26 | 5-methyl-1,2,4-oxadiazol-3-yl | 2,5-Cl₂ | 544.3 | 1.68 | 5 |
| P1.27 | 5-methyl-1,2,4-oxadiazol-3-yl | 3-F, 4-Cl | 516.3 | 1.75 | 5 |
| P1.28 | 5-methyl-1,2,4-oxadiazol-3-yl | 2-Cl, 4-Br | 516.3 | 1.74 | 5 |
| P1.29 | 5-methyl-1,2,4-oxadiazol-3-yl | 2-F, 5-Br | 500.32 | 1.66 | 5 |
| P1.30 | 5-methyl-1,2,4-oxadiazol-3-yl | 3-Cl, 4-F | 560.28 | 1.82 | 5 |
| P1.31 | 5-methyl-1,2,4-oxadiazol-3-yl | 2,4,5-F₃ | 502.35 | 1.58 | 5 |
| P1.32 | 5-methyl-1,2,4-oxadiazol-3-yl | 3,4-F₂ | 484.34 | 1.56 | 5 |
| P1.33 | 5-methyl-1,2,4-oxadiazol-3-yl | 3-Br | 526.3 | 1.67 | 5 |
| P1.34 | 5-methyl-1,2,4-oxadiazol-3-yl | 2-F, 4-Cl | 500.32 | 1.68 | 5 |
| P1.35 | 5-methyl-1,2,4-oxadiazol-3-yl | 3-cyclopropyl | 488.4 | 1.74 | 5 |
| P1.36 | 5-methyl-1,2,4-oxadiazol-3-yl | 2-F, 4-Br | 544.31 | 1.72 | 5 |
| P1.37 | 5-methyl-1,2,4-oxadiazol-3-yl | 3-Br, 4-Cl | 560.28 | 1.79 | 5 |
| P1.38 | 5-methyl-1,2,4-oxadiazol-3-yl | 2-Cl, 5-Br | 560.28 | 1.79 | 5 |
| P1.39 | 5-methyl-1,2,4-oxadiazol-3-yl | 3-Cl, 4-Br | 560.28 | 1.79 | 5 |
| P1.40 | 5-methyl-1,2,4-oxadiazol-3-yl | 4-Br | 526.29 | 1.69 | 5 |
| P1.41 | 1-methylpyrazol-3-yl | 3-F, 4-Br | 543 | 1.07 | 1 |
| P1.42 | 5-methyl-1,2,4-oxadiazol-3-yl | 2-F, 5-CHF₂ | 516 | 1.16 | 3 |
| P1.43 | 5-methyl-1,2,4-oxadiazol-3-yl | 2-F, 4-OCH₃ | 496 | 1.06 | 1 |
| P1.44 | 1-methylpyrazol-3-yl | 2-F, 5-Cl | 498 | 1.07 | 1 |
| P1.45 | 5-methyl-1,2,4-oxadiazol-3-yl | 2-Br | 526/528 | 1.51 | 4 |
| P1.46 | 5-methyl-1,2,4-oxadiazol-3-yl | 4-I | 574 | 1.11 | 1 |
| P1.47 | 1-methylpyrazol-3-yl | 2-Cl, 5-Br | 558 | 1.1 | 1 |
| P1.48 | 1-methylpyrazol-3-yl | 2-F, 4-Cl | 498 | 1.07 | 1 |
| P1.49 | 5-methyl-1,2,4-oxadiazol-3-yl | 4-CHF₂ | 498 | 1.05 | 1 |
| P1.50 | 5-methyl-1,2,4-oxadiazol-3-yl | 2-Br, 5-OCH₃ | 555.98 | 1.63 | 4 |
| P1.51 | 5-methyl-1,2,4-oxadiazol-3-yl | 2-Br, 4-F | 544.22 | 1.62 | 4 |
| P1.52 | 5-methyl-1,2,4-oxadiazol-3-yl | 2-Cl, 4,5-F₂ | 518.26 | 1.63 | 4 |
| P1.53 | 5-methyl-1,2,4-oxadiazol-3-yl | 2-Br, 4,5-F₂ | 562.22 | 1.65 | 4 |
| P1.54 | 5-methyl-1,2,4-oxadiazol-3-yl | 3-OCH₃, 4-SO₂CH₃ | 556.32 | 1.19 | 4 |
| P1.55 | 5-methyl-1,2,4-oxadiazol-3-yl | 2-F, 4-OCH₃ | 496.3 | 1.49 | 4 |
| P1.56 | 5-methyl-1,2,4-oxadiazol-3-yl | 2-Br, 5-Cl | 560.18 | 1.7 | 4 |
| P1.57 | 5-methyl-1,2,4-oxadiazol-3-yl | 2,3,6-Cl₃ | 550.19 | 1.72 | 4 |
| P1.58 | 5-methyl-1,2,4-oxadiazol-3-yl | 2-O—CF₂—O-3 | 528.29 | 1.61 | 4 |
| P1.59 | 1-methylpyrazol-3-yl | 2,4,5-F₃ | 500 | 1.13 | 3 |
| P1.60 | 5-methyl-1,1-dioxo-1,2,5- | 2-F, 5-Cl | 552 | 1.14 | 3 |
| | thiadiazolidin-2-yl) | | | | |
| P1.61 | 5-methyl-1,1-dioxo-1,2,5- | 2-Cl, 5-Br | 612 | 1.09 | 1 |
| | thiadiazolidin-2-yl) | | | | |
| P1.62 | 5-methyl-1,1-dioxo-1,2,5- | 2-F, 4-Cl | 552 | 1.13 | 3 |
| | thiadiazolidin-2-yl) | | | | |
| P1.63 | 5-methyl-1,2,4-oxadiazol-3-yl | 4-OCHF₂ | 514 | 1.06 | 1 |
| P1.64 | 5-methyl-1,2,4-oxadiazol-3-yl | 2-Br, 5-Cl | 560 | 1.13 | 1 |

### Table P2: Compounds of Formula (I-d)

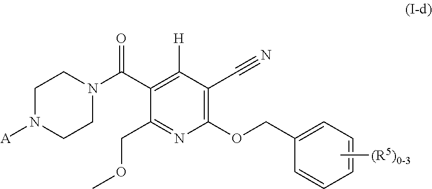

The compounds in Table P2 can be prepared as described in the examples above or similar methodology. The following abbreviations are used in the table below: RT=retention time, min=minutes.

TABLE P2

| Comp. | | | | LC-MS | |
|---|---|---|---|---|---|
| No | A | R⁵ | [M + H]⁺ | RT (min) | Method |
| P2.1 | 3,5-dichloro-pyridine-2-yl | 3-O—CF₂—O-4 | 592 | 1.33 | 3 |
| P2.2 | isopropylsulfonyl | 3-O—CF₂—O-4 | 553 | 1.09 | 1 |
| P2.3 | methylsulfamoyl | 3-O—CF₂—O-4 | 540 | 1.14 | 3 |
| P2.4 | ethylsulfamoyl | 3-F, 4-Br | 570 | 1.08 | 1 |
| P2.5 | ethylsulfamoyl | 2,4,5-F₃ | 528 | 1.05 | 1 |
| P2.6 | ethylsulfamoyl | 2-F, 5-Cl | 526 | 1.15 | 3 |
| P2.7 | ethylsulfamoyl | 2-F, 4-Cl | 526 | 1.19 | 3 |
| P2.8 | propylsulfonyl | 2-Cl, 4-O—CF₂—O-5 | 587 | 1.24 | 3 |
| P2.9 | propylsulfonyl | 3-F, 4-Br | 569 | 1.08 | 1 |
| P2.10 | propylsulfonyl | 3-O—CF₂—O-4 | 553 | 1.09 | 1 |
| P2.11 | ethylsulfamoyl | 2-Cl, 5-Br | 586 | 1.17 | 3 |

### Table P3: Compounds of Formula (I-e)

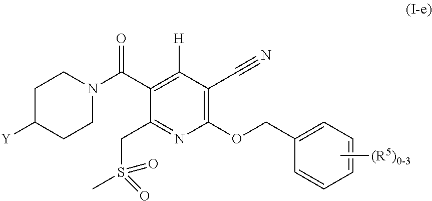

The compound in Table P3 can be prepared as described in the examples above or similar methodology. The following abbreviations are used in the table below: RT=retention time, min=minutes.

TABLE P3

| Comp. | | | | LC-MS | |
|---|---|---|---|---|---|
| No | Y | R⁵ | [M + H]⁺ | RT (min) | Method |
| P3.1 | 5-methyl-1,2,4-oxadiazol-3-yl | 3-O—CF₂—O-4 | 576 | 1.12 | 3 |

### Table P4: Compounds of Formula (I-f)

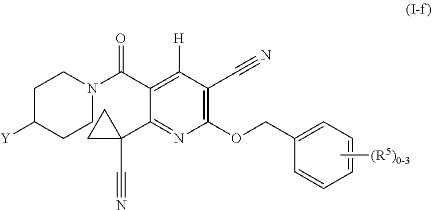

The compound in Table P4 can be prepared as described in the examples above or similar methodology. The following abbreviations are used in the table below: RT=retention time, min=minutes.

TABLE P4

| Comp. | | | | LC-MS | |
|---|---|---|---|---|---|
| No | Y | R⁵ | [M + H]⁺ | RT (min) | Method |
| P4.1 | 5-methyl-1,2,4-oxadiazol-3-yl | 3-O—CF₂—O-4 | 549 | 1.07 | 6 |

### Table P5: Compounds of Formula (I-q)

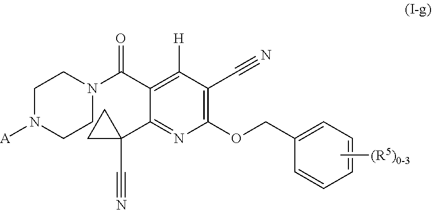

The compound in Table P5 can be prepared as described in the examples above or similar methodology. The following abbreviations are used in the table below: RT=retention time, min=minutes.

TABLE P5

| Comp. | | | | LC-MS | |
|---|---|---|---|---|---|
| No | Y | R⁵ | [M + H]⁺ | RT (min) | Method |
| P5.1 | 3,5-dichloro-pyridine-2-yl | 3-O—CF₂—O-4 | 613 | 1.26 | 6 |

### Table P6: Compounds of Formula (I-h)

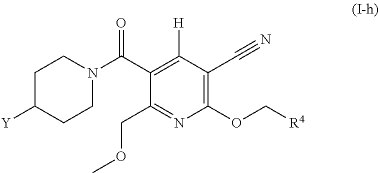

The compounds in Table P6 can be prepared as described in the examples above or similar methodology. The following abbreviations are used in the table below: RT=retention time, min=minutes.

TABLE P6

| Comp. | | | | LC-MS | |
|---|---|---|---|---|---|
| No | Y | R⁴ | [M + H]⁺ | RT (min) | Method |
| P6.1 | 5-methyl-1,2,4- | 3-chloro-2-quinolyl | 533 | 1.15 | 3 |
| | oxadiazol-3-yl | | | | |
| P6.2 | 5-methyl-1,2,4- | 6,7-dichloroimidazo[1,2- | 556 | 1.06 | 1 |
| | oxadiazol-3-yl | a]pyridine-2-yl | | | |
| P6.3 | 5-methyl-1,2,4- | 4,6-dichloro-3-pyridyl | 517.31 | 1.52 | 5 |
| | oxadiazol-3-yl | | | | |
| P6.4 | 5-methyl-1,2,4- | 2,1,3-benzoxadiazol-5-yl | 490.35 | 1.45 | 5 |
| | oxadiazol-3-yl | | | | |
| P6.5 | 5-methyl-1,2,4- | 5-bromothiazol-2-yl | 533.25 | 1.43 | 5 |
| | oxadiazol-3-yl | | | | |
| P6.6 | 5-methyl-1,2,4- | 4-fluoro-1,3-benzoxazol-2-yl | 507.37 | 1.42 | 5 |
| | oxadiazol-3-yl | | | | |
| P6.7 | 5-methyl-1,2,4- | 3-cyclopropylisoxazol-5-yl | 479.35 | 1.35 | 5 |
| | oxadiazol-3-yl | | | | |
| P6.8 | 5-methyl-1,2,4- | 6-quinolyl | 499.39 | 1.13 | 5 |
| | oxadiazol-3-yl | | | | |
| P6.9 | 5-methyl-1,2,4- | 5-bromo-2-pyridyl | 527.3 | 1.44 | 5 |
| | oxadiazol-3-yl | | | | |
| P6.10 | 5-methyl-1,2,4- | 1-chloro-3-isoquinolyl | 533.37 | 1.66 | 5 |
| | oxadiazol-3-yl | | | | |
| P6.11 | 5-methyl-1,2,4- | 5-chlorothiazol-2-yl | 489.29 | 1.42 | 5 |
| | oxadiazol-3-yl | | | | |
| P6.12 | 5-methyl-1,2,4- | 2-cyclopropylthiazol-4-yl | 495.36 | 1.44 | 5 |
| | oxadiazol-3-yl | | | | |
| P6.13 | 5-methyl-1,2,4- | pyrazolo[1,5-a]pyridin-2-yl | 488.38 | 1.34 | 5 |
| | oxadiazol-3-yl | | | | |
| P6.14 | 5-methyl-1,2,4- | 5-fluoro-1,3-benzoxazol-2-yl | 507.38 | 1.41 | 5 |
| | oxadiazol-3-yl | | | | |
| P6.15 | 5-methyl-1,2,4- | 6,8-dichloroimidazo[1,2- | 556.33 | 1.45 | 5 |
| | oxadiazol-3-yl | a]pyridin-2-yl | | | |
| P6.16 | 5-methyl-1,2,4- | 7-chloro-2-quinolyl | 533.37 | 1.61 | 5 |
| | oxadiazol-3-yl | | | | |
| P6.17 | 5-methyl-1,2,4- | 2,4-dichlorothiazol-5-yl | 523.25 | 1.56 | 5 |
| | oxadiazol-3-yl | | | | |
| P6.18 | 5-methyl-1,2,4- | 2-quinolyl | 499.39 | 1.4 | 5 |
| | oxadiazol-3-yl | | | | |
| P6.19 | 5-methyl-1,2,4- | 5-ethylsulfonyl-1,3- | 581.39 | 1.26 | 5 |
| | oxadiazol-3-yl | benzoxazol-2-yl | | | |
| P6.20 | 5-methyl-1,2,4- | 2,6-dichloro-3-quinolyl | 567.34 | 1.77 | 5 |
| | oxadiazol-3-yl | | | | |
| P6.21 | 5-methyl-1,2,4- | 6-chloro-5-fluoro-3-pyridyl | 501.32 | 1.47 | 5 |
| | oxadiazol-3-yl | | | | |
| P6.22 | 5-methyl-1,2,4- | 3-quinolyl | 499.39 | 1.28 | 5 |
| | oxadiazol-3-yl | | | | |
| P6.23 | 5-methyl-1,2,4- | 2,5-dichloro-3-thienyl | 522.26 | 1.82 | 5 |
| | oxadiazol-3-yl | | | | |
| P6.24 | 5-methyl-1,2,4- | 1,3-benzothiazol-5-yl | 505.35 | 1.4 | 5 |
| | oxadiazol-3-yl | | | | |
| P6.25 | 5-methyl-1,2,4- | 2,1,3-benzothiadiazol-5-yl | 506.35 | 1.5 | 5 |
| | oxadiazol-3-yl | | | | |
| P6.26 | 5-methyl-1,2,4- | thiazolo[5,4-b]pyridin-2-yl | 506.35 | 1.27 | 5 |
| | oxadiazol-3-yl | | | | |
| P6.27 | 5-methyl-1,2,4- | 7-chloro-1,3-benzoxazol-2-yl | 523.35 | 1.54 | 5 |
| | oxadiazol-3-yl | | | | |
| P6.28 | 5-methyl-1,2,4- | 6-fluoro-1,3-benzoxazol-2-yl | 507.37 | 1.44 | 5 |
| | oxadiazol-3-yl | | | | |
| P6.29 | 5-methyl-1,2,4- | 5-chloro-1,3,4-thiadiazol-2-yl | 490.27 | 1.25 | 5 |
| | oxadiazol-3-yl | | | | |
| P6.30 | 5-methyl-1,2,4- | 4-cyclopropylthiazol-2-yl | 495.36 | 1.48 | 5 |
| | oxadiazol-3-yl | | | | |
| P6.31 | 5-methyl-1,2,4- | 5-chloro-1,3-benzothiazol-2- | 539.32 | 1.66 | 5 |
| | oxadiazol-3-yl | yl | | | |
| P6.32 | 5-methyl-1,2,4- | 6-bromoimidazo[1,2- | 566.32 | 1.16 | 5 |
| | oxadiazol-3-yl | a]pyridin-2-yl | | | |
| P6.33 | 5-methyl-1,2,4- | 8-bromoimidazo[1,2- | 566.32 | 1.2 | 5 |
| | oxadiazol-3-yl | a]pyridin-2-yl | | | |
| P6.34 | 5-methyl-1,2,4- | 2-chlorooxazol-4-yl | 473.29 | 1.33 | 5 |
| | oxadiazol-3-yl | | | | |
| P6.35 | 5-methyl-1,2,4- | imidazo[1,2-a]pyridin-2-yl | 488.37 | 0.77 | 5 |
| | oxadiazol-3-yl | | | | |
| P6.36 | 5-methyl-1,2,4- | 5-bromo-1,3,4-thiadiazol-2-yl | 534.24 | 1.26 | 5 |
| | oxadiazol-3-yl | | | | |
| P6.37 | 5-methyl-1,2,4- | 6-bromo-3-pyridyl | 527.29 | 1.41 | 5 |
| | oxadiazol-3-yl | | | | |
| P6.38 | 5-methyl-1,2,4- | 5-bromo-1-methyl- | 580.34 | 1.39 | 5 |
| | oxadiazol-3-yl | benzimidazol-2-yl | | | |
| P6.39 | 5-methyl-1,2,4- | 2-chloro-3-quinolyl | 533.35 | 1.61 | 5 |
| | oxadiazol-3-yl | | | | |
| P6.40 | 5-methyl-1,2,4- | 3,6,7-trichloroimidazo[1,2- | 590 | 1.13 | 3 |
| | oxadiazol-3-yl | a]pyridin-2-yl | | | |
| P6.41 | 5-methyl-1,2,4- | 2,3-dichloro-7-fluoro- | 586 | 1.19 | 3 |
| | oxadiazol-3-yl | quinoxalin-6-yl | | | |
| P6.42 | 5-methyl-1,2,4- | 1,3-benzoxazol-6-yl | 489 | 1.07 | 3 |
| | oxadiazol-3-yl | | | | |
| P6.43 | 5-methyl-1,2,4- | 1,3-benzoxazol-5-yl | 489 | 1.07 | 3 |
| | oxadiazol-3-yl | | | | |
| P6.44 | 5-methyl-1,2,4- | 6-chloro-2-pyridyl | 483.26 | 1.35 | 5 |
| | oxadiazol-3-yl | | | | |
| P6.45 | 5-methyl-1,2,4- | 3-chloro-4-pyridyl | 483.27 | 1.27 | 5 |
| | oxadiazol-3-yl | | | | |
| P6.46 | 5-methyl-1,2,4- | 1-chloro-7-isoquinolyl | 533.28 | 1.48 | 5 |
| | oxadiazol-3-yl | | | | |
| P6.47 | 5-methyl-1,2,4- | 2-methoxy-1,3-benzoxazol-5- | 519.32 | 1.29 | 5 |
| | oxadiazol-3-yl | yl | | | |
| P6.48 | 5-methyl-1,2,4- | 3-chloro-7-methoxy- | 552.28 | 1.12 | 5 |
| | oxadiazol-3-yl | imidazo[1,2-a]pyridin-2-yl | | | |
| P6.49 | 5-methyl-1,2,4- | 7-fluoro-2,3-dimethoxy- | 578.35 | 1.63 | 5 |
| | oxadiazol-3-yl | quinoxalin-6-yl | | | |
| P6.50 | 5-methyl-1,2,4- | 3,7-dichloroimidazo[1,2- | 556.24 | 1.41 | 5 |
| | oxadiazol-3-yl | a]pyridin-2-yl | | | |
| P6.51 | 5-methyl-1,1-dioxo- | 2,1,3-benzoxadiazol-5-yl | 542 | 1.01 | 1 |
| | 1,2,5-thiadiazolidin- | | | | |
| | 2-yl) | | | | |
| P6.52 | 1-methylpyrazol-3- | 2,1,3-benzoxadiazol-5-yl | 488 | 1.02 | 1 |
| | yl | | | | |
| P6.53 | ethylsulfamoyl | 2,1,3-benzoxadiazol-5-yl | 516 | 1.09 | 3 |
| P6.54 | 5-methyl-1,2,4- | 7-chloroquinoxaline-6-yl | 534 | 0.99 | 1 |
| | oxadiazol-3-yl | | | | |

The activity of the compositions according to the invention can be broadened considerably, and adapted to prevailing circumstances, by adding other insecticidally, acaricidally and/or fungicidally active ingredients. The mixtures of the compounds of formula (I) with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridinylmethyleneamino derivatives, macrolides, neonicotinoids and Bacillus thuringiensis preparations.

The following mixtures of a compound of formula (I) with an active substances are preferred (the abbreviation “TX” means “one compound selected from the compounds defined in Tables 1 to 81 and Tables P1 to P6”):
an adjuvant selected from the group of substances consisting of petroleum oils (alternative name) (628)+TX;
an insect control active substance selected from abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: 1305319-70-3+TX, CAS number: 1442448-92-1+TX, CAS number: RNA (Leptinotarsa decemlineata-specific recombinant double-stranded interfering GS2)+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX;
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B2 (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B2+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, 0-5-dichloro-4-iodophenyl O-ethyl ethylphosphonothioate+TX, O,O-diethyl 0-4-methyl-2-oxo-2H-chromen-7-yl phosphorothioate+TX, O,O-diethyl 0-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, -metalaxyl-+TX, Rmetalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-11H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate (may be prepared from the methods described in WO 2020/056090)+TX, ethyl 1-[[4-[(Z)-2-ethoxy-3,3,3-trifluoro-prop-1-enoxy]phenyl]methyl]pyrazole-3-carboxylate (may be prepared from the methods described in WO 2020/056090)+TX, methyl N-[[4-[1-(4-cyclopropyl-2,6-difluoro-phenyl)pyrazol-4-yl]-2-methyl-phenyl]methyl]carbamate (may be prepared from the methods described in WO 2020/097012)+TX, methyl N-[[4-[1-(2,6-difluoro-4-isopropyl-phenyl)pyrazol-4-yl]-2-methyl-phenyl]methyl]carbamate (may be prepared from the methods described in WO 2020/097012)+TX, 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391)+TX, 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391)+TX, 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391)+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1, 1-dimethylethyl)-α-[4′-(trifluoromethoxy) [1, 1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®);
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate;
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator;
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;
(1) antibacterial agents selected from the group of:
(1.1) bacteria, examples of which are Bacillus mojavensis strain R3B (Accession No. NCAIM (P) B001389) (WO 2013/034938) from Certis USA LLC, a subsidiary of Mitsui & Co.+TX; Bacillus pumilus, in particular strain BU F-33, having NRRL Accession No. 50185 (available as part of the CARTISSA® product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661, U.S. Pat. No. 6,060,051)+TX; Bacillus subtilis strain BU1814, (available as VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC, a subsidiary of Mitsui & Co.+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX+TX; Bacillus licheniformis FMCH001 and Bacillus subtilis FMCHOO2 (QUARTZO® (WG) and PRESENCE® (WP) from FMC Corporation)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC, a subsidiary of Mitsui & Co.+TX; Bacillus mycoides, isolate, having Accession No. B-30890 (available as BMJ TGAI® or WG and LifeGard™ from Certis USA LLC, a subsidiary of Mitsui & Co.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BSO3 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), or strain IK726 (Jensen D F, et al. Development of a biocontrol agent for plant disease control with special emphasis on the near commercial fungal antagonist Clonostachys rosea strain ‘IK726’, Australas Plant Pathol. 2007,36:95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Dèrivés), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (having Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICC080 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO® 7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1 (e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. and BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB-SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens μm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BPO1 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (having Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (having Accession Nos. NRRL B-50421 and described in U.S. patent application Ser. No. 13/330,576)+TX; Bacillus subtilis, in particular strain AQ30004 (and NRRL B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCH002 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and
(3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel from Agrimm Technologies Limited)+TX; Trichoderma atroviride strain SC1 described in International Application No. PCT/IT2008/000196)+TX; Trichoderma asperellum strain kd (e.g. T-Gro from Andermatt Biocontrol)+TX; Trichoderma asperellum strain Eco-T (Plant Health Products, ZA), Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. comprised in Myco-Sol from Helena Chemical Company)+TX; Rhizopogon fulvigleba (e.g. comprised in Myco-Sol from Helena Chemical Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC, a subsidiary of Mitsui & Co.)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. Colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 from Becker Microbial Products, IL+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11, (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX from Certis, US)+TX; Bacillus thuringiensis subsp. kurstaki strain EG 7841 (CRYMAX from Certis, US)+TX; Bacillus thuringiensis subsp. tenebrionis strain NB 176 (SD-5428, e.g. NOVODOR® FC from BioFa DE)+TX; Brevibacillus laterosporus (LATERAL from Ecolibrium Biologicals)+TX; Burkholderia spp., in particular Burkholderia rinojensis strain A396 (also known as Burkholderia rinojensis strain MBI 305) (Accession No. NRRL B-50319+TX; WO 2011/106491 and WO 2013/032693+TX; e.g. MB1206 TGAI and ZELTO® from Marrone Bio Innovations)+TX; Chromobacterium subtsugae, in particular strain PRAA4-1T (MBI-203+TX; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ and MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam International Corporation)+TX; Beauveria bassiana strain ATP02 (Accession No. DSM 24665)+TX; Isaria fumosorosea (previously known as Paecilomyces fumosoroseus) strain Apopka 97) PREFERAL from SePRO+TX; Metarhizium anisopliae 3213-1 (deposited under NRRL accession number 67074) (WO 2017/066094+TX; Pioneer Hi-Bred International)+TX; Metarhizium robertsii 15013-1 (deposited under NRRL accession number 67073)+TX; Metarhizium robertsii 23013-3 (deposited under NRRL accession number 67075)+TX; Paecilomyces lilacinus strain 251 (MELOCON from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray+TX; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Heads Up (Chenopodium quinoa saponin extract)+TX; PROBLAD (naturally occurring Blad polypeptide from Lupin seeds), Certis EU+TX; FRACTURE (naturally occurring Blad polypeptide from Lupin seeds), FMC+TX; Pyrethrum/Pyrethrins+TX; Quassia amara+TX; Quercus+TX; Quillaja extract (QL AGRI 35 from BASF)+TX; Reynoutria sachalinensis extract (REGALLIA/REGALIA MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula (I) selected from the compounds defined in the Tables 1 to 81 and Tables P1 to P6 with active ingredients described above comprises a compound selected from one compound defined in the Tables 1 to 81 and Tables P1 to P6 and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from the compounds defined in the Tables 1 to 81 and Tables P1 to P6 and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of formula (I) of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I. Further, it is hereby made available, a composition comprising a plant propagation material treated with a compound of formula I.

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 24 ppm, 12.5 ppm, 6 ppm, 3 ppm, 1.5 ppm, 0.8 ppm or 0.2 ppm.

### Example B1: Tetranychus urticae (Two-Spotted Spider Mite)

Feeding/contact activity Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P1.1, P1.2., P1.3, P1.4, P1.5, P1.6, P1.7, P1.8, P1.9, P1.10, P1.11, P1.12, P1.13, P1.14, P1.15, P1.18, P1.20, P1.22, P1.23, P1.24, P1.25, P1.26, P1.27, P1.28, P1.29, P1.30, P1.31, P1.32, P1.34, P1.36, P1.37, P1.38, P1.39, P1.40, P1.41, P1.43, P1.44, P1.46, P1.47, P1.48, P1.49, P1.51, P1.52, P1.53, P1.55, P1.56, P1.59, P1.60, P1.61, P1.62, P2.1, P2.2, P2.3, P2.4, P2.5, P2.6, P2.7, P2.8, P2.9, P2.10, P2.11, P6.1, P6.3, P6.4, P6.7, P6.11, P6.16, P6.21, P6.24, P6.31, P6.37, P6.38, P6.41, P6.50, P6.51, P6.52, P6.53.

1. A compound of the formula (I) 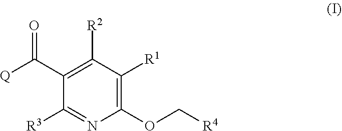
wherein
R¹ is CN or C(═S)NH₂;
R² is H, OH, halogen, C₁-C₆-alkoxy or C₁-C₆-haloalkoxy;
R³ is C₁-C₆-alkoxy-C₁-C₆-alkyl, C₁-C₆-haloalkoxy-C₁-C₆-alkyl, C₂-C₆-alkenyloxy-C₁-C₆-alkyl, C₂-C₆-haloalkenyloxy-C₁-C₆-alkyl, C₂-C₆-alkynyloxy-C₁-C₆-alkyl, C₂-C₆-haloalkynyloxy-C₁-C₆-alkyl, C₃-C₆-cycloalkoxy-C₁-C₆-alkyl, C₃-C₆-halocycloalkoxy-C₁-C₆-alkyl, C₁-C₆-alkylsulfonyl-C₁-C₆-alkyl; C₁-C₆-alkylsulfonyl-C₃-C₆-cycloalkyl, C₁-C₆-cyanoalkyl, or C₃-C₆-cyanocycloalkyl;
R⁴ is C₁-C₆-haloalkyl, C₂-C₆-alkenyl, C₂-C₆-haloalkenyl, C₃-C₆-cycloalkyl, C₃-C₆-halocycloalkyl, phenyl, phenyl substituted with 1 to 3 independently selected substituents R⁵, heteroaryl (which is either a 5 or 6 membered monocyclic or a 8, 9 or 10 membered bicyclic), heteroaryl (which is either a 5 or 6 membered monocyclic or a 8, 9 or 10 membered bicyclic) substituted with 1 to 3 independently selected substituents R⁶, phenyl-C₁-C₃-alkyl, or phenyl-C₁-C₃-alkyl substituted with 1 to 3 substituents independently selected R⁷;
Q is a cyclic amine represented by the formula IIa or a cyclic amine represented by the formula IIb, 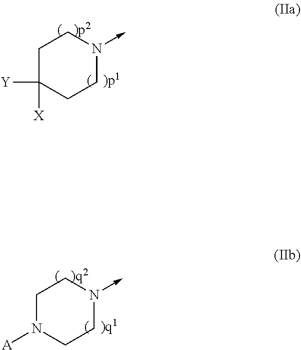
wherein the arrow indicates the connection to the carbonyl group;
p¹ is 0, 1 or 2 and indicates the number of methylene groups;
p² is 0, 1 or 2 and indicates the number of methylene groups;
q¹ is 1 or 2 and indicates the number of methylene groups;
q² is 1 or 2 and indicates the number of methylene groups;
X is hydrogen, hydroxyl, alkoxy, or halogen;
Y is cyano, C₁-C₆-alkyl, C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, C₃-C₆-alkenylsulfanyl-C₁-C₆-alkyl, C₃-C₆-alkenylsulfinyl-C₃-C₆-alkyl, C₃-C₆-alkenylsulfonyl-C₁-C₆-alkyl, C₃-C₆-alkynylsulfanyl-C₁-C₆-alkyl, C₃-C₆-alkynylsulfinyl-C₁-C₆-alkyl, C₁-C₆-alkynylsulfonyl-C₁-C₆-alkyl, R^{a}R^{b}NC(O), R^{c}C(O)NR^{d}, R^{e}SO₂NR^{f}, R^{g}O—N═CR^{h}, 4 to 6 membered non-aromatic heterocyclic ring system in which one or two carbons are replaced independently by nitrogen, oxygen, sulfur, or sulfonyl, phenyl, phenyl substituted with 1 to 3 independently selected substituents R⁸, 5 or 6 membered monocyclic heteroaryl, or 5 or 6 membered monocyclic heteroaryl substituted with 1 to 3 independently selected substituents R⁹;
A is cyano, C₁-C₆-cyanoalkyl, C₂-C₆-cyanoalkenyl, C₃-C₆-cyanocycloalkyl, C₁-C₆-haloalkyl, C₁-C₆-haloalkenyl, C₃-C₆-halocycloalkyl, C₁-C₆-alkoxycarbonyl, C₂-C₆-alkenyloxycarbonyl, C₂-C₆-alkynyloxycarbonyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, RⁱSO₂, R^{j}R^{k}NSO₂, phenyl, phenyl substituted with 1 to 3 independently selected substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 independently selected substituents R¹¹;
R^{a}, R^{b}, R^{c}, R^{d}, R^{f}, R^{g}, R^{h}, R^{j} and R^{k} are independently selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl and C₃-C₆-halocycloalkyl;
R^{e} and Rⁱ are independently selected from C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl and C₃-C₆-halocycloalkyl;
R⁵ is independently selected from halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₁-C₆-alkylsulfonyl, C₁-C₆-haloalkylsulfanyl and —O—C₁₋₂haloalkanediyl-O—; and
R⁶, R⁷, R⁸, R⁹, R¹⁰, R¹¹ are independently selected from halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₁-C₆-alkylsulfonyl, and C₁-C₆-haloalkylsulfanyl;
or an agrochemically acceptable salt, stereoisomer, enantiomer, tautomer and N-oxide of the compound of formula (I).

2. The compound according to claim 1 wherein R¹ is CN.

3. The compound according to either claim 1 or claim 2 wherein R² is H.

4. The compound according to any one of claims 1 to 3 wherein R³ C₁-C₄-alkoxy-C₁-C₃-alkyl, C₁-C₄-haloalkoxy-C₁-C₃-alkyl, C₂-C₄-alkenyloxy-C₁-C₃-alkyl, C₂-C₄-haloalkenyloxy-C₁-C₃-alkyl, C₂-C₄-alkynyloxy-C₁-C₃-alkyl, C₂-C₄-haloalkynyloxy-C₁-C₃-alkyl, C₃-C₆-cycloalkoxy-C₁-C₃-alkyl, C₃-C₆-halocycloalkoxy-C₁-C₃-alkyl, C₁-C₄-alkylsulfonyl-C₁-C₃-alkyl; C₁-C₄-alkylsulfonyl-C₃-C₆-cycloalkyl, C₁-C₄-cyanoalkyl, or C₃-C₆-cyanocycloalkyl.

5. The compound according to any one of claims 1 to 4 wherein R⁴ is C₁-C₆-haloalkyl, C₂-C₆-alkenyl, C₂-C₆-haloalkenyl, C₃-C₆-cycloalkyl, C₃-C₆-halocycloalkyl, phenyl, phenyl substituted with 1 to 3 substituents R⁵, 5 or 6 membered monocyclic heteroaryl, 5 or 6 membered monocyclic heteroaryl substituted with 1 to 3 substituents R⁶, phenyl-C₁-C₃-alkyl, or phenyl-C₁-C₃-alkyl substituted with 1 to 3 substituents R⁷, wherein R⁵, R⁶ and R⁷ are as defined in claim 1.

6. The compound according to any one of claims 1 to 5 wherein R⁴ is phenyl substituted with 1 to 3 substituents R⁵.

7. The compound according to any one of claims 1 to 6 wherein Q is a cyclic amine represented by the formula IIa, wherein both p¹ and p² are 1; X is hydrogen, hydroxyl, alkoxy, or halogen; and Y is cyano, C₁-C₆-alkyl, C₁-C₆-alkoxy-C₁-C₆-alkyl, C₁-C₆-alkylsulfanyl-C₁-C₆-alkyl, C₁-C₆-alkylsulfinyl-C₁-C₆-alkyl, C₁-C₆-alkylsulfonyl-C₁-C₆-alkyl, R^{a}R^{b}NC(O), R^{c}C(O)NR^{d}, R^{e}SO₂NR^{f}, R^{g}O—N═CR^{h}, 4 to 6 membered non-aromatic heterocyclic ring system in which one or two carbons is replaced independently by nitrogen, oxygen, sulfur, or sulfonyl, phenyl, phenyl substituted with 1 to 3 substituents R⁸, a 5 or 6 membered monocyclic heteroaryl, or a 5 or 6 membered monocyclic heteroaryl substituted with 1 to 3 substituents R⁹, wherein R^{a}, R^{b}, R^{c}, R^{d}, R^{e}, R^{f}, R^{g}, R^{h}, R⁸ and R⁹ are as defined in claim 1.

8. The compound according to any one of claims 1 to 6 wherein Q is a cyclic amine represented by the formula IIb, wherein both q¹ and q² are 1; and A is cyano, C₁-C₆-cyanoalkyl, C₂-C₆-cyanoalkenyl, C₃-C₆-cyanocycloalkyl, C₁-C₆-haloalkyl, C₁-C₆-haloalkenyl, C₃-C₆-halocycloalkyl, C₁-C₆-alkoxycarbonyl, C₂-C₆-alkenyloxycarbonyl, C₂-C₆-alkynyloxycarbonyl, RⁱSO₂, R^{j}R^{k}NSO₂, phenyl, phenyl substituted with 1 to 3 substituents R¹⁰, heteroaryl (which is either a 5 or 6 membered monocyclic or a 8, 9 or 10 membered bicyclic), or heteroaryl (which is either a 5 or 6 membered monocyclic or a 8, 9 or 10 membered bicyclic) substituted with 1 to 3 substituents R¹¹, wherein R′, R^{j},
R^{k}, R¹⁰ and R¹¹ are as defined in claim 1.

9. A composition comprising a compound as defined in any one of claims 1 to 8, one or more auxiliaries and diluent, and optionally one more other active ingredient.

10. A method
(i) of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined as defined in any one of claims 1 to 8 or a composition as defined claim 9; or
(ii) for the protection of plant propagation material from the attack by insects, acarines, nematodes or molluscs, which comprises treating the propagation material or the site, where the propagation material is planted, with an effective amount of a compound as defined in any one of claims 1 to 8 or a composition as defined claim 9; or
(iii) of controlling parasites in or on an animal in need thereof comprising administering an effective amount of a compound as defined in any one of claims 1 to 8 or a composition as defined claim 9.

11. A plant propagation material, such as a seed, comprising, or treated with or adhered thereto, a compound as defined in any one of claims 1 to 8 or a composition as defined claim 9.

12. A compound of formula III-a 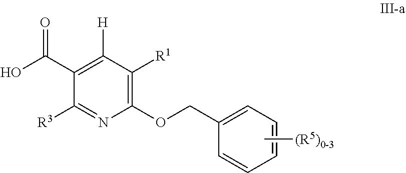
wherein R¹, R³ and R⁵ are defined in claim 1;
a compound of formula V-a 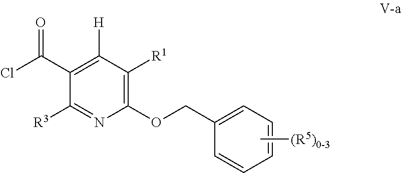
wherein R¹, R³ and R⁵ are defined in claim 1; or
a compound of formula VI-a 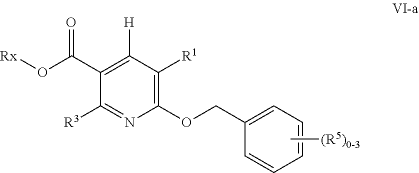
wherein R¹, R³, and R⁵ are defined in claim 1, and R^{x} is selected from methyl, ethyl, propyl, iso-propyl, butyl, iso-butyl and tert-butyl.

13. A compound of formula VII-a 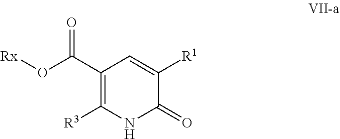
wherein R¹ and R³ are defined in claim 1, and R^{x} is selected from methyl, ethyl, propyl, iso-propyl, butyl, iso-butyl and tert-butyl;
a compound of formula IX-a 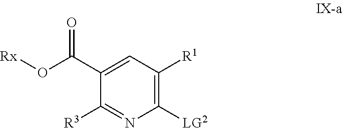
wherein R¹ and R³ are defined in claim 1, R^{x} is selected from methyl, ethyl, propyl, iso-propyl, butyl, iso-butyl and tert-butyl, and LG² is chloro; or
a compound of XIV-a 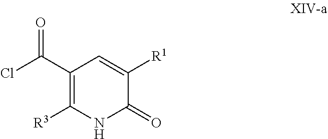
wherein R¹ is defined in claim 1 and R³ is methoxymethyl.

14. A compound of formula XI-a 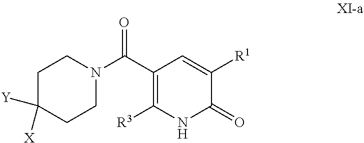
wherein R¹, R³, X and Y are defined in claim 1;
a compound of XI-b 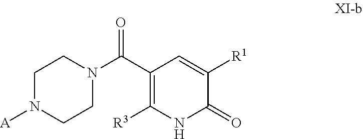
wherein R¹, R³, and A are defined in claim 1;
a compound of XII-a 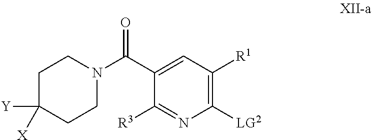
wherein R¹, R³, X and Y are defined in claim 1, and LG² is chloro, bromo or fluoro; or
a compound of XII-b 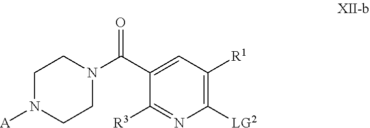
wherein R¹, R³, and A are defined claim 1, and LG² is chloro, bromo or fluoro.
